(19) United States
(12) Patent Application Publication
(10) Pub. No.: US 2016/0193024 A1
(43) Pub. Date: Jul. 7, 2016

(54) MEDICAL TUBE ASSEMBLY AND PUNCTURE APPARATUS

(71) Applicant: TERUMO KABUSHIKI KAISHA, Tokyo (JP)

(72) Inventors: Nao MANDAI, Mountain View, CA (US); Yuusuke TAKAHASHI, Ashigarakami-gun (JP)

(73) Assignee: TERUMO KABUSHIKI KAISHA, Tokyo (JP)

(21) Appl. No.: 15/067,819

(22) Filed: Mar. 11, 2016

Related U.S. Application Data
(63) Continuation of application No. PCT/JP2014/074110, filed on Sep. 11, 2014.

(30) Foreign Application Priority Data
Sep. 21, 2013 (JP) 2013-196266

(51) Int. Cl. A61F 2/00; A61M 25/06

(52) U.S. Cl. CPC A61F 2/0045; A61M 25/06; A61M 2210/1092

(57) ABSTRACT

A medical tube assembly is disclosed, which includes a medical tube having a tubular main body in which an implant to be placed indwelling in a living body is insertable, an insertion section having a curved portion and being elongated, an interlock portion interlocking a distal portion of the medical tube and a distal portion of the insertion section in a freely detachable manner, and a rotation restriction unit restricting rotation of the medical tube and the insertion section around an axis. When inserted into a living body with the distal portion of the medical tube and the distal portion of the insertion section interlocked to each other, the insertion section and the main body are inserted in an aligned state.

### CROSS-REFERENCES TO RELATED APPLICATIONS

This application is a continuation of International Application No. PCT/JP2014/074110 filed on Sep. 11, 2014, and claims priority to Japanese Application No. 2013-196266 filed on Sep. 21, 2013, the entire content of both of which is incorporated herein by reference.

### TECHNICAL FIELD

The present disclosure generally relates to a medical tube assembly and a puncture apparatus.

### BACKGROUND DISCUSSION

If a person suffers from a urinary incontinence, specifically, for example, if a person suffers from a stress urinary incontinence, then urine leakage can be caused by application of abdominal pressure during normal exercise or by laughing, coughing, sneezing or the like. The cause of this may be, for example, that the pelvic floor muscle which is a muscle for supporting the urethra is loosened by birth or the like.

For the treatment of urinary incontinence, a surgical treatment can be effective, in which there is used, for example, a belt-shaped implant called “sling.” The sling is indwelled inside the body and the urethra is supported by the sling (see, for example, Japanese Patent Laid-open No. 2010-99499). In order to indwell the sling inside the body, an operator would incise the vagina with a surgical knife, dissect the part between the urethra and vagina, and make the dissected region and the outside communicate with each other through obturator foramens by use of a puncture needle or the like. Then, in this state, the sling is indwelled into the body.

If the vaginal wall is incised once, however, a situation may occur that the sling is exposed to the inside of the vagina from a wound caused by the incision of the vaginal wall, and complications may be caused by an infection from the wound or the like. Further, since the vaginal wall is incised, there is such a defect that the invasion is great and the burden on the patient is heavy. Further, the urethra may be damaged by a surgical knife in the course of the procedure by the operator. In addition, the fingertip of the operator himself/herself may be damaged or injured by the surgical knife.

As a method for solving the above-mentioned problem, a method may be considered in which a sling is placed indwelling in a living body, without incision of the vaginal wall, by using a medical tube assembly that includes a medical tube and a curved elongated insertion part inserted in the medical tube. In this method, first, the living body is punctured from a body surface by a needle body provided at a distal portion of the medical tube assembly, and the needle body is caused to pass between a urethra and a vagina and again protrude from the body surface to the exterior of the body (this operation will hereinafter referred to as “puncturing operation”). As a result of the puncturing operation, an intermediate part of the medical tube assembly is located between the urethra and the vagina, and both ends of the medical tube assembly each protrude from the body surface to the exterior of the body. Subsequently, the insertion part is drawn out of the medical tube, and the sling is placed indwelling in the living body through the medical tube.

In the medical tube assembly as above, however, since the medical tube assembly is used with the insertion part inserted in the medical tube, the length of the medical tube is set to be not more than the length of the insertion part. Therefore, in the case of application of the medical tube assembly to a relatively large patient, for example, it may be difficult to place the medical tube in such a manner that an intermediate part of the medical tube is located between the urethra and the vagina and both ends of the medical tube protrude from the body surface to the exterior of the body. Thus, the medical tube assembly may not be applicable for use with a relatively large patient.

### SUMMARY

Accordingly, a medical tube assembly and a puncture apparatus are disclosed by which an implant can be placed indwelling in a living body relatively easily and reliably in the cases of patients of various body types.

A medical tube assembly is disclosed, which includes a medical tube having a tubular main body in which an implant to be placed indwelling in a living body is insertable; an insertion section having a curved portion and being elongated; an interlock portion interlocking a distal portion of the medical tube and a distal portion of the insertion section in a freely detachable manner; and a rotation restriction unit restricting rotation of the medical tube and the insertion section around an axis, wherein when inserted into a living body with the distal portion of the medical tube and the distal portion of the insertion section interlocked to each other, the insertion section and the main body are inserted in an aligned state.

In accordance with an exemplary embodiment, the medical tube assembly is configured such that the length of the main body in an axial direction is greater than the length of the insertion section in an axial direction.

In accordance with an exemplary embodiment, the medical tube assembly is configured such that the rotation restriction unit is provided at distal portions of the main body and the insertion section.

In accordance with an exemplary embodiment, the medical tube assembly is configured such that the rotation restriction unit is provided at side surfaces of the main body and the insertion section.

In accordance with an exemplary embodiment, the medical tube assembly is configured such that the main body has a curved portion, and the main body, in a state of being inserted in a living body, is rigid in such a manner as to be able to maintain curvature and an internal cavity of the main body.

In accordance with an exemplary embodiment, the medical tube assembly may further include a needle body capable of puncturing a living body at a distal portion of the medical tube assembly.

A puncture apparatus is disclosed, which includes a medical tube assembly disposed to be rotationally movable, the medical tube assembly including a medical tube having a tubular main body in which an implant to be placed indwelling in a living body is insertable, an insertion section having a curved portion and being elongated, an interlock portion interlocking a distal portion of the medical tube and a distal portion of the insertion section in a freely detachable manner, a rotation restriction unit restricting rotation of the medical tube and the insertion section around an axis, and a needle body capable of puncturing a living body at a distal portion of the medical tube assembly, when inserted into a living body with the distal portion of the medical tube and the distal portion of the insertion section interlocked to each other, the insertion section and the main body being inserted in an aligned state; a urethral-insertion member to be inserted into a urethra, the urethral-insertion member elongated in shape; and a restriction unit restricting positional relationship of the medical tube assembly and the urethral-insertion member in such a manner that when the medical tube assembly is rotationally moved for puncturing a biological tissue, a needle point of the needle body passes on a farther side from a center of rotary movement of the medical tube assembly than the urethral-insertion member.

In accordance with an exemplary embodiment, the puncture apparatus is configured such that the main body is flat shaped in cross section, and the rotation restriction unit restricts the rotation in such a manner that a major axis in cross section of the main body and an axis of the urethral-insertion member will be parallel to each other.

In accordance with an exemplary embodiment, the medical tube assembly has the interlock section, which interlocks a distal portion of the medical tube and a distal portion of the insertion section, and the rotation restriction unit. By interlocking the distal portion of the medical tube and the distal portion of the insertion section, therefore, the procedure can be carried out without inserting the insertion section in the medical tube. As a result, the length of the medical tube can be set to be greater than the length of the insertion section, and the medical tube assembly can be applied also to a fat patient, for example. In other words, an implant can be placed indwelling in a living body easily and assuredly, in the cases of patients of various body types.

### BRIEF DESCRIPTION OF THE DRAWINGS

FIG. 1 is a perspective view showing a puncture apparatus to which a medical tube assembly according to a first embodiment of the present disclosure is applied;

FIG. 2 is a side view of the puncture apparatus shown in FIG. 1;

FIG. 3 is a plan view showing an operating member possessed by the puncture apparatus shown in FIG. 1;

FIGS. 4A and 4B illustrate a puncture member possessed by the puncture apparatus shown in FIG. 1, wherein FIG. 4A is a perspective view and FIG. 4B is a sectional view taken along line IVB-IVB of FIG. 4A;

FIG. 5 is a sectional view of the puncture member shown in FIG. 4A;

FIGS. 6A to 6C illustrate a state maintaining mechanism possessed by the puncture member shown in FIG. 4A, wherein FIG. 6A is a top plan view, and FIGS. 6B and 6C are sectional views;

FIGS. 7A to 7C show a partial enlarged view depicting a state maintaining mechanism possessed by the puncture member shown in FIG. 4A, wherein FIGS. 7A and 7B are each plan views showing modifications, and FIG. 7C is a plan view showing the present embodiment;

FIGS. 8A and 8B illustrate a second anchor possessed by the puncture apparatus shown in FIG. 1, wherein FIG. 8A is a sectional view, and FIG. 8B is a sectional view showing a condition where the second anchor is engaged with the puncture member;

FIGS. 9A and 9B illustrate a first anchor possessed by the puncture apparatus shown in FIG. 1, wherein FIG. 9A is a sectional view, and FIG. 9B is a sectional view showing a condition where the first anchor is engaged with the puncture member;

FIG. 10 is a sectional view showing a guide section of a frame possessed by the puncture apparatus shown in FIG. 1;

FIG. 11 is a sectional view showing the guide section of the frame possessed by the puncture apparatus shown in FIG. 1;

FIG. 12 is a sectional view showing the guide section of the frame possessed by the puncture apparatus shown in FIG. 1;

FIG. 13 is a plan view showing a fixing section of the frame possessed by the puncture apparatus shown in FIG. 1;

FIG. 14 is a side view of an insertion tool possessed by the puncture apparatus shown in FIG. 1;

FIGS. 15A and 15B illustrate a positional relation of the puncture member and an obturator foramen (pelvis), wherein FIG. 15A is a side view and FIG. 15B is a front view;

FIG. 16 is a partial enlarged view of a vaginal-insertion member possessed by the insertion tool shown in FIG. 14;

FIG. 17A is a sectional view showing an example of the shape of a vaginal wall, and FIG. 17B is a sectional view showing a state where a vaginal-insertion section is inserted in a vagina shown in FIG. 17A;

FIG. 18 illustrates an implant to be used with the puncture apparatus shown in FIG. 1;

FIGS. 19A and 19B are each views for explaining an operating procedure of the puncture apparatus shown in FIG. 1;

FIGS. 20A and 20B are views for explaining the operating procedure of the puncture apparatus shown in FIG. 1;

FIG. 21 is a side view showing the relation between the puncture apparatus and the pelvis at the time of the state shown in FIG. 20A;

FIGS. 22A and 22B are each views for explaining the operating procedure of the puncture apparatus shown in FIG. 1;

FIG. 23 is a side view showing the relation between the puncture apparatus and the pelvis at the time of the state shown in FIG. 22A;

FIG. 24 is a sectional view showing the posture of the puncture member relative to a urethra at the time of the state shown in FIG. 22B;

FIGS. 25A and 25B are each views for explaining the operating procedure of the puncture apparatus shown in FIG. 1;

FIGS. 26A and 26B are each views for explaining the operating procedure of the puncture apparatus shown in FIG. 1;

FIG. 27 is a perspective view showing a medical tube assembly according to a second embodiment of the present disclosure;

FIG. 28 is a sectional view showing a modification of the medical tube assembly shown in FIG. 27;

FIG. 29 is a plan view showing a medical tube assembly according to a third embodiment of the present disclosure;

FIG. 30 is a plan view showing a medical tube assembly according to a fourth embodiment of the present disclosure;

FIG. 31 is a plan view showing a medical tube assembly according to a fifth embodiment of the present disclosure;

FIG. 32 is a plan view showing a medical tube assembly according to a sixth embodiment of the present disclosure;

FIG. 33 is a plan view showing a medical tube assembly according to a seventh embodiment of the present disclosure;

FIG. 34 is a plan view showing a medical tube assembly according to an eighth embodiment of the present disclosure;

FIG. 35 is a plan view showing a medical tube assembly according to a ninth embodiment of the present disclosure;

FIG. 36 is a plan view showing a medical tube assembly according to a tenth embodiment of the present disclosure;

FIG. 37 is a plan view showing a medical tube assembly according to an eleventh embodiment of the present disclosure;

FIG. 38 is a side view showing a distal portion of the medical tube assembly shown in FIG. 37;

FIG. 39 is a sectional view taken along line XXXIX-XXXIX of FIG. 38;

FIG. 40 is a view for explaining a use example of the medical tube assembly shown in FIG. 37;

FIG. 41 is a view for explaining the use example of the medical tube assembly shown in FIG. 37;

FIG. 42 is a view for explaining the use example of the medical tube assembly shown in FIG. 37;

FIG. 43 is a view for explaining the use example of the medical tube assembly shown in FIG. 37;

FIG. 44 is a view for explaining the use example of the medical tube assembly shown in FIG. 37;

FIG. 45 is a side view showing a distal portion of a medical tube assembly according to a twelfth embodiment of the present disclosure;

FIG. 46 is a sectional view taken along line XLV-XLV of FIG. 45;

FIG. 47 is a sectional view showing an intermediate part of a medical tube assembly according to a thirteenth embodiment of the present disclosure;

FIG. 48 is a sectional view showing an intermediate part of a medical tube assembly according to a fourteenth embodiment of the present disclosure;

FIG. 49 is a sectional view showing a medical tube (medical tube assembly) according to a fifteenth embodiment of the present disclosure;

FIG. 50 is a view for explaining a use example of the medical tube (medical tube assembly) shown in FIG. 49;

FIG. 51 is a view for explaining the use example of the medical tube (medical tube assembly) shown in FIG. 49;

FIG. 52 is a view for explaining the use example of the medical tube (medical tube assembly) shown in FIG. 49;

FIGS. 53A and 53B are sectional views showing a medical tube (medical tube assembly) according to a sixteenth embodiment of the present disclosure;

FIGS. 54A and 54B are views for explaining a use example of the medical tube (medical tube assembly) shown in FIGS. 53A and 53B;

FIG. 55 is a sectional view showing a medical tube (medical tube assembly) according to a seventeenth embodiment of the present disclosure;

FIG. 56 is a view for explaining a use example of the medical tube (medical tube assembly) shown in FIG. 55;

FIGS. 57A and 57B are views for explaining the use example of the medical tube (medical tube assembly) shown in FIG. 55;

FIGS. 58A and 58B are views for explaining the use example of the medical tube (medical tube assembly) shown in FIG. 55;

FIGS. 59A and 59B are perspective views showing a medical tube according to an eighteenth embodiment of the present disclosure;

FIG. 60 is a view for explaining a use example of the medical tube shown in FIGS. 59A and 59B;

FIG. 61 is a sectional view taken along line LXI-LXI of FIG. 59B;

FIG. 62 is a perspective view showing a medical tube according to a nineteenth embodiment of the present disclosure;

FIG. 63 is a view for explaining a use example of the medical tube shown in FIG. 62;

FIG. 64 is a sectional view showing a medical tube according to a twentieth embodiment of the present disclosure; and

FIG. 65 is a perspective view showing a medical tube according to a twenty-first embodiment of the present disclosure.

### DETAILED DESCRIPTION

A medical tube assembly and a puncture apparatus according to the described aspects of the present disclosure will be described in detail below, referring to preferred embodiments illustrated in the attached drawings.

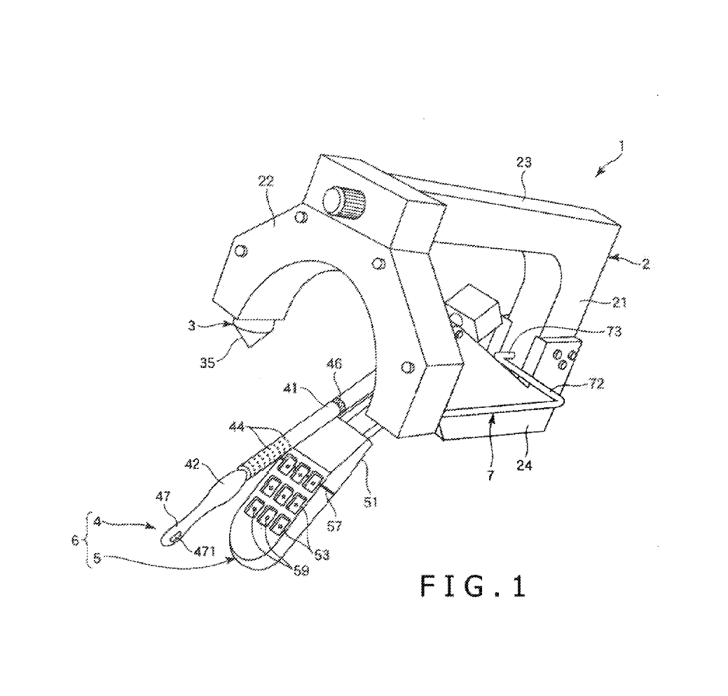

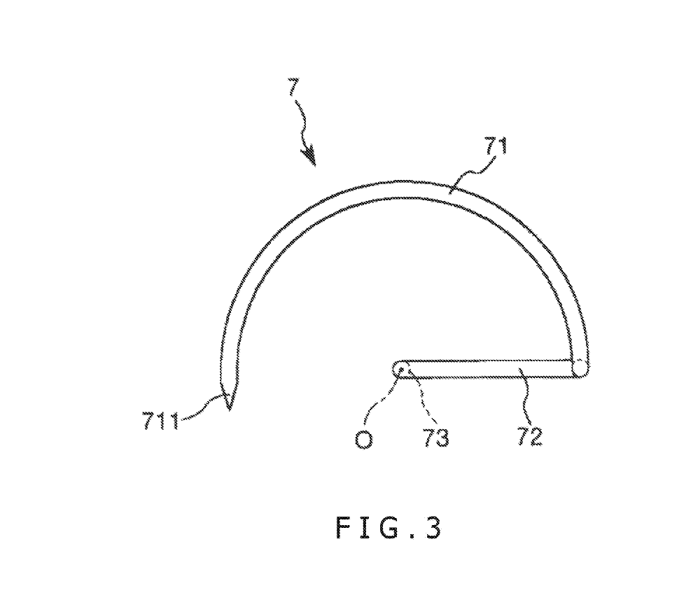

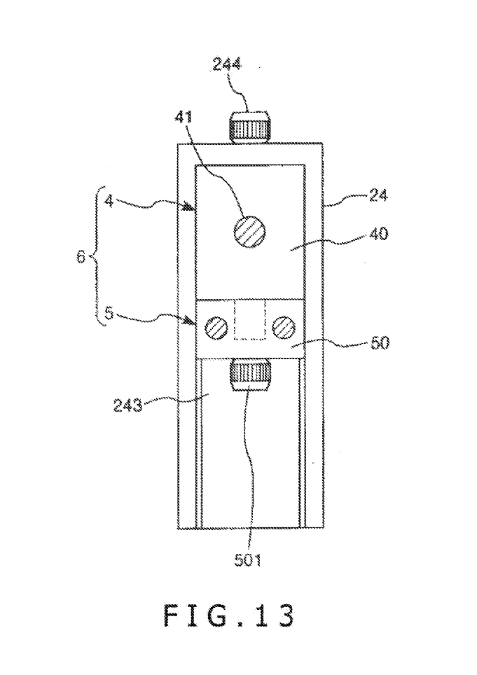

First, a puncture apparatus according to a first embodiment is described below with reference to FIGS. 1 to 26B.

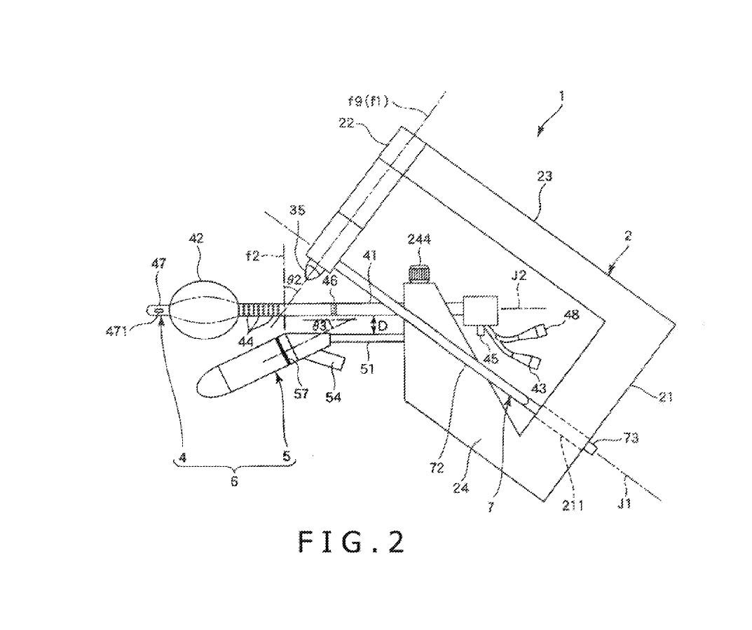

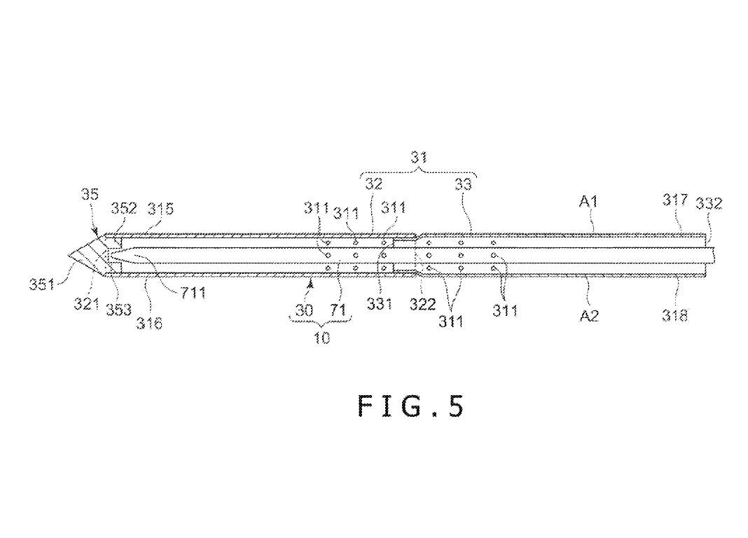

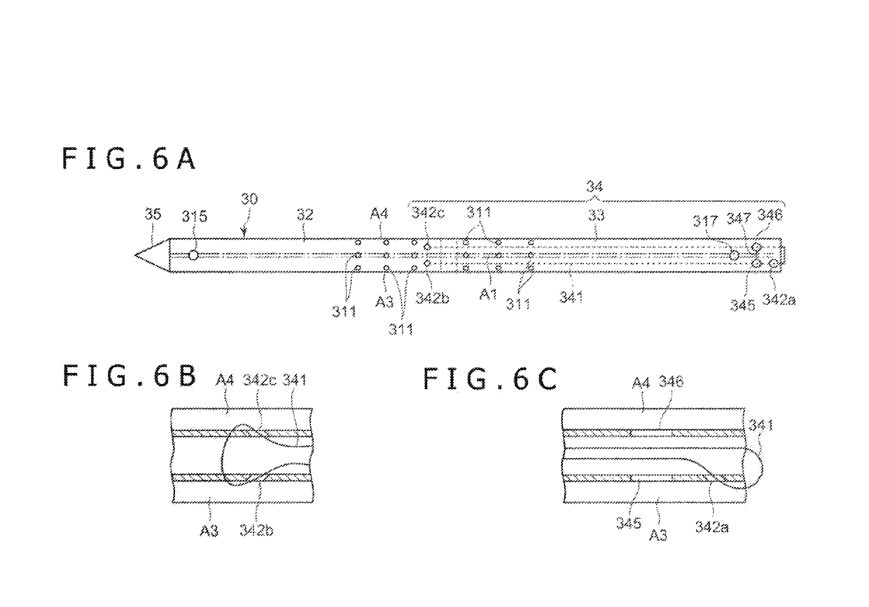

Note that in the following, for convenience of explanation, the left side in FIG. 2 will be referred to as “distal side,” the right side as “proximal side,” the upper side as “upper side,” and the lower side as “lower side.” FIG. 2 shows a puncture apparatus in the state of being not yet used, and this state will be referred to also as the “initial state” for convenience of explanation. In addition, a state where the puncture apparatus (insertion tool) shown in FIG. 2 is mounted onto a patient will be referred to also as the “mounted state.” Further, in each of FIGS. 5, 6A, 6B, and 6C, a puncture member extending in a circular arc shape is depicted in the state of being stretched rectilinearly, for convenience of explanation.

First, a puncture apparatus to which a medical tube assembly (a medical tube) of the present disclosure are applied will be described.

A puncture apparatus 1 shown in FIGS. 1 and 2 is an apparatus for use in treatment of female urinary incontinence, specifically, for example, in embedding (implanting) in a living body a biological tissue supporting indwelling article for treatment of urinary incontinence.

This puncture apparatus 1 can include a frame (support section) 2, a puncture member 3, a urethral-insertion member 4, a vaginal-insertion member 5, an operating member 7 and anchors 81 and 82. The puncture member 3, the urethral-insertion member 4, the vaginal-insertion member 5, the operating member 7 and the anchors 81 and 82 are each supported by the frame 2. In accordance with an exemplary embodiment, in the puncture apparatus 1, the urethral-insertion member 4 and the vaginal-insertion member 5 constitute an insertion tool 6. These will be sequentially described below.

The operating member 7 is a member for operating the puncture member 3. As shown in FIGS. 1 to 3, such an operating member 7 can include an insertion section 71, a shaft section 73, and an interlock section 72 interlocking the insertion section 71 and the shaft section 73. The insertion member 71, the interlock section 72 and the shaft section 73 may be formed to be integral with one another, or, alternatively, at least one of them may be formed as a separate body from the others of them.

The insertion section 71 is a part to be inserted into the puncture member 3, and functions as a stylet for reinforcing the puncture member 3 internally. With the insertion section 71 inserted in the puncture member 3, the puncture member 3 is connected to the operating member 7, whereby an operation of the puncture member 3 by the operating member 7 can be enabled. The insertion member 71 as above has a circular arc shape corresponding to the shape of the puncture member 3. The center angle of the insertion member 71 is set in accordance with the center angle of the puncture member 3. A distal portion 711 of the insertion section 71 is tapered off With the tapered-off distal portion 711 provided, the insertion of the insertion section 71 into the puncture member 3 can be performed relatively smoothly.

Note that while the insertion section 71 is circular in cross-sectional shape, the insertion section 71 may be flat shaped in cross section. The flat shape here is not specifically restricted; for example, ellipses, rhombuses with corners rounded, rectangles (flat shapes) with corners rounded, and spindle-like shapes with a central portion enlarged (enlarged in diameter) as compared with both end portions may be adopted as the flat shape.

The shaft section 73 extends along an axis J1 which intersects the center O of the insertion section 71 and is orthogonal to a plane f1 containing the insertion section 71.

The interlock section 72 interlocks a proximal portion of the insertion section 71 and a distal portion of the shaft section 73. In accordance with an exemplary embodiment, the interlock section 72 has a substantially L-shaped form of being bent substantially at right angle at an intermediate portion thereof. The interlock section 72 functions also as a grip section to be gripped by an operator at the time of operating the operating member 7.

In accordance with an exemplary embodiment, the operating member 7 as above is configured to be higher in rigidity than the puncture member 3 (main body 31). The material constituting the operating member 7 is not particularly limited; there can be used, for example, various metallic materials such as stainless steel, aluminum or aluminum alloys, and titanium or titanium alloys.

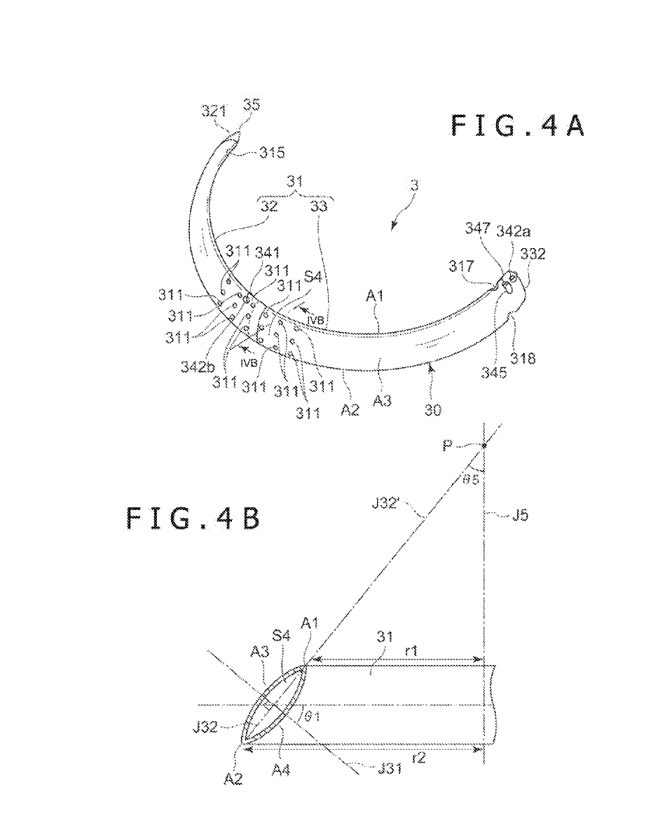

The puncture member 3 is a member for puncturing a living body. As shown in FIG. 4A, such a puncture member 3 can include an elongated sheath (medical tube) 30, and a needle body 35 provided at the distal end of the sheath 30. The sheath 30 can include the main body 31, which is tubular in shape, and a state maintaining mechanism 34.

The main body 31 can include an elongated tubular body (tube), opening at both the distal end and the proximal end thereof. Such a main body 31 has an internal space in which an implant main body 91 can be inserted. Note that the internal space can include an internal cavity of the main body 31. The main body 31 has a bent shape of being bent in a circular arc shape, and is flat shaped in cross section as shown in FIG. 4B. In accordance with an exemplary embodiment, the cross-sectional shape at a central portion S4 in the longitudinal direction of the main body 31 is a flat shape including a minor axis J31 and a major axis J32. As will be described later, the implant main body 91 is disposed inside the main body 31. With the main body 31 flat shaped, therefore, the posture of the implant main body 91 within the main body 31 can be controlled.

In addition, the width (the length in the direction of the major axis J32) of the internal space of the main body 31 is designed to be substantially the same as the width of a main body section 911 (described later) of the implant main body 91, which helps ensure that even when the implant main body 91 is moved, the frictional resistance with the internal space of the main body 31 is low, so that no unnecessary force is exerted on the implant main body 91, and the main body section 911 can be disposed in a sufficiently developed state within the main body 31. Note that the width (the length in the direction of the major axis J32) of the internal space of the main body 31 may be shorter than the width of the main body section 911. As a result, the width of the main body 31 can be restrained from becoming large, so that a less invasive puncture member 3 can be realized.

Note that the flat shape of the main body 31 is not specifically restricted; for example, ellipses, convex lens-like shapes in section, rhombuses with corners rounded, rectangles (flat shapes) with corners rounded, and spindle-like shapes with a central portion enlarged (enlarged in diameter) as compared with both end portions may be adopted as the flat shape.

In the following, for convenience of explanation, an end portion located on the inner side (one end) in the direction of the major axis J32 will be referred to also as an “inner circumferential portion A1,” an end portion located on the outer side (other end) will be referred to also as an “outer circumferential portion A2,” a surface oriented toward the upper side will be referred to also as a “front surface A3,” and a surface oriented toward the lower side will be referred to also as a “back surface A4,” as shown in FIG. 4B.

As shown in FIG. 4B, let a plane containing both the center point of the circular arc of the central portion S4 and the center point of the cross-sectional shape with respect to the longitudinal direction of the main body 31 (a plane containing the center axis of the main body 31) be a plane f9, and let the angle formed between the plane f9 and the minor axis J31 at the central portion S4 be an inclination angle θ1, then the inclination angle θ1 is preferably, for example, an acute angle. With the inclination angle θ1 set to be an acute angle, an implant 9 (described later) can be disposed substantially in parallel to the urethra, so that the urethra can be supported more effectively. This effect will be described in detail later.

Note that the inclination angle θ1 is preferably, for example, about 20° to 60°, more preferably 30° to 45°, and further preferably about 35° to 40°. This causes a further enhancement of the aforementioned effect.

While it is preferable that the inclination angle θ1 satisfies the aforementioned numerical range over the whole region in the extending direction of the main body 31, the above-mentioned effect can be exhibited if the aforementioned numerical range is satisfied at least at the central portion S4 in the extending direction of the main body 31. Note that the “central portion S4” means a region including the part located between the urethra and the vagina at least in a state where a living body is punctured by the puncture member 3 (a state where the main body 31 is disposed inside the living body). In addition, in this embodiment, it can be also said that a central portion (the center and the vicinity on both sides of the same) between the anchors 81 and 82, in a state where the anchors 81 and 82 are in engagement with the puncture member 33 as will be described later, is the central portion S4.

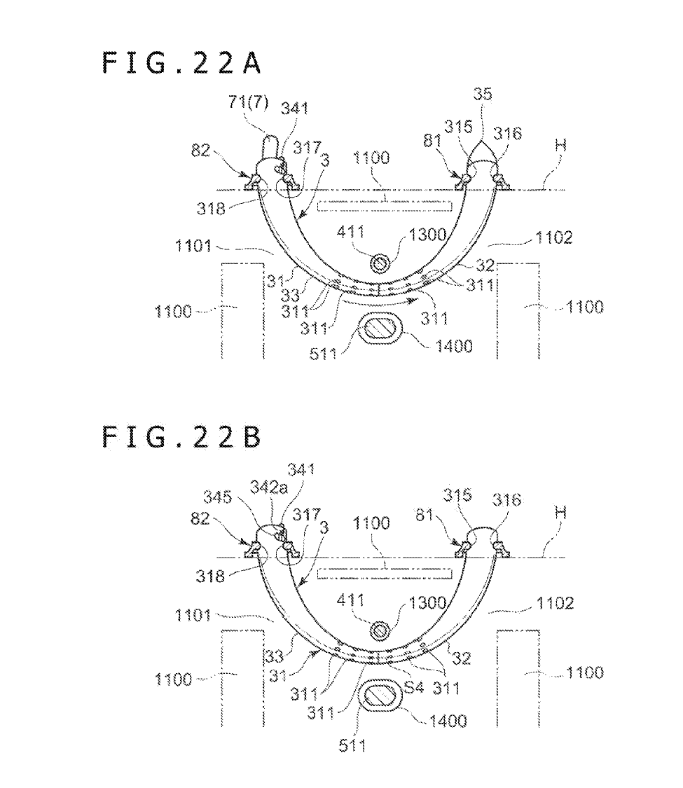

Note that both end portions of the main body 31 may be provided with markers at parts which are located equidistantly from the central portion S4 and which protrude to the outside of a living body in a state where the main body 31 is disposed in the living body (the state shown in FIGS. 22A and 22B), which helps ensure that the position of the central portion S4 inside the living body can be confirmed by comparing the positions of both the markers.

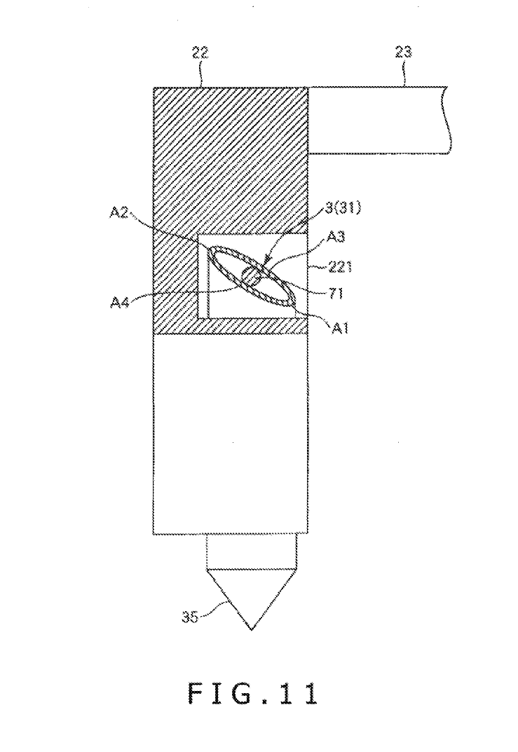

The configuration of the main body 31 can be described in other words as follows. It can be also said that as shown in FIG. 4B, the main body 31 is so formed that the major axis J32 is inclined against the center axis J5 of the circular arc and that the center axis J5 of the circular arc and an extension line J32′ of the major axis J32 have an intersection P. In this case, the angle θ5 formed between the center axis J5 and the extension line J32′ is equal to the inclination angle θ1. In addition, it can be also said that as shown in FIG. 10, in plan view as viewed from the direction of the center axis J5 of the main body 31, the main body 31 has the inner circumferential portion A1 located at its inner circumferential edge and having a minimum radius of curvature r1 and the outer circumferential portion A2 located at its outer circumferential edge and having a maximum radius of curvature r2, and, as shown in FIG. 4B, the inner circumferential portion A1 and the outer circumferential portion A2 are located to be spaced from each other in the direction of the center axis J5.

The main body 31 thus shaped is composed of two separable pieces (separable tubes) so that it can be divided at an intermediate portion thereof. Specifically, the main body 31 is divided into a distal separable piece (first separable tube) 32 and a proximal separable piece (second separable tube) 33. The distal separable piece 32 and the proximal separable piece 33 are substantially the same in length, and the boundary between the two separable pieces is located in the central portion S4.

As shown in FIG. 5, the distal separable piece 32 is tubular in shape, and has a distal-side opening 321 and a proximal-side opening 322. In addition, the proximal separable piece 33 is tubular in shape, and has a distal-side opening 331 and a proximal-side opening 332. A distal portion of the proximal separable piece 33 is inserted in a proximal portion of the distal separable piece 32, whereby the distal separable piece 32 and the proximal separable piece 33 are connected with each other. With the proximal separable piece 33 thus inserted in the distal separable piece 32, a step which could be generated at the boundary between the separable pieces 32 and 33 is unlikely to be caught on the biological tissue, so that puncture of a living body by the puncture member 3 can be performed relatively smoothly. Note that contrary to this embodiment, the distal separable piece 32 may be inserted in the proximal separable piece 33 to thereby connect the separable pieces 32 and 33 together.

The connected state in which these separable pieces 32 and 33 are connected together is maintained by the state maintaining mechanism 34. As shown in FIG. 6A, the state maintaining mechanism 34 can include holes 342a, 342b and 342c, an endless string (interlock member) 341 inserted in and passed through the holes 342a, 342b and 342c, exposure holes (through-holes) 345 and 346 for exposure of the string 341, and a slit 347 interconnecting the exposure holes 345 and 346.

In accordance with an exemplary embodiment, the hole 342a is provided in a proximal portion of the proximal separable piece 33 at a position near the inner circumferential portion A1 of the front surface A3. In accordance with an exemplary embodiment, the holes 342b and 342c are provided in a proximal portion of the distal separable piece 32, oppositely at positions which are in the front surface A3 and the back surface A4 and which are near the inner circumferential portion A1.

The string 341 can be disposed inside the main body 31, while being exposed outside of the main body 31 between the holes 342b and 342c and between the hole 342a and the proximal-side opening 332. With the string 341 laid around in this manner, the connected state of the separable pieces 32 and 33 can be maintained. In addition, the degree of exposure of the string 341 outside of the main body 31 can be lowered, so that the string 341 is less likely to be caught on the biological tissue. In addition, the overall length of the string 341 can be made as short as possible, while enabling the string 341 to be cut as will be described later. Therefore, the string 341 is less likely to be caught on the implant main body 91 at the time of inserting and passing the implant main body 91 into and through the main body 31. Further, since the holes 342a, 342b and 342c are disposed near the inner circumferential portion A1 as aforementioned, the string 341 is also disposed near the inner circumferential portion A1. Therefore, the string 341 is less likely to be caught on the implant main body 91 at the time of inserting the implant main body 91 into the main body 31.

The string 341 as above can be obtained, for example, by a method wherein a string having ends is prepared, one end of the string is inserted into the main body 31 via the proximal-side opening 332, is drawn out to the outside of the main body 31 through the hole 342b, is inserted into the main body 31 via the hole 342c, is drawn out to the outside of the main body 31 through the hole 342a, and, finally, is tied with the other end of the string in the vicinity of the proximal-side opening 332. It is to be noted, however, that the position of the knot is not limited.

Here, as shown in FIG. 6C, the axis of the hole 342a is inclined so that the outside opening is located on the proximal side as compared with the inside opening. In addition, as shown in FIG. 6B, the axis of each of the holes 342b and 342c is inclined so that the outside opening is located on the distal side as compared with the inside opening, which helps ensure that each of the holes 342a, 342b and 342c can be extended along the path of the string 341, so that the string 341 is less likely to be caught on each of the holes 342a, 342b and 342c.

The exposure holes 345 and 346 are oppositely provided in the front surface A3 and the back surface A4 of the proximal portion of the proximal separable piece 33. The part where the exposure holes 345 and 346 are provided protrudes from a body surface in a state where the main body 31 is disposed inside a living body. In addition, the exposure holes 345 and 346 are located on the path of the string 341. Therefore, the string 341 is exposed outside of the main body 31 via the exposure holes 345 and 346. In accordance with an exemplary embodiment, these exposure holes 345 and 346 are interconnected by the slit 347 provided in the inner circumferential portion A1 along the circumferential direction of the main body 31.

In the state maintaining mechanism 34 as above, cutting the string 341 results in a state in which the distal separable piece 32 and the proximal separable piece 33 are separable from each other. This configuration helps enable the distal separable piece 32 and the proximal separable piece 33 to be put into a separable state through a simple operation. In addition, since the cutting of the string 341 is visible, it can be easily confirmed that the distal separable piece 32 and the proximal separable piece 33 have been put into the separable state.

With the exposure holes 345 and 346 and the slit 347 provided as in this embodiment, the string 341 can be cut easily. Referring to one example, scissors including a pair of blades (a first blade and a second blade) are prepared, the first blade is inserted into and passed through the exposure holes 345 and 346, and the string 341 is positioned between the pair of blades. Then, the scissors are put into a closing operation, whereby at least one of the first and second blades is passed through the slit 347, and the first and second blades come to overlap with each other, in which process the string 341 is cut. Thus, where the exposure holes 345 and 346 and the slit 347 are provided, the string 341 can be easily cut.

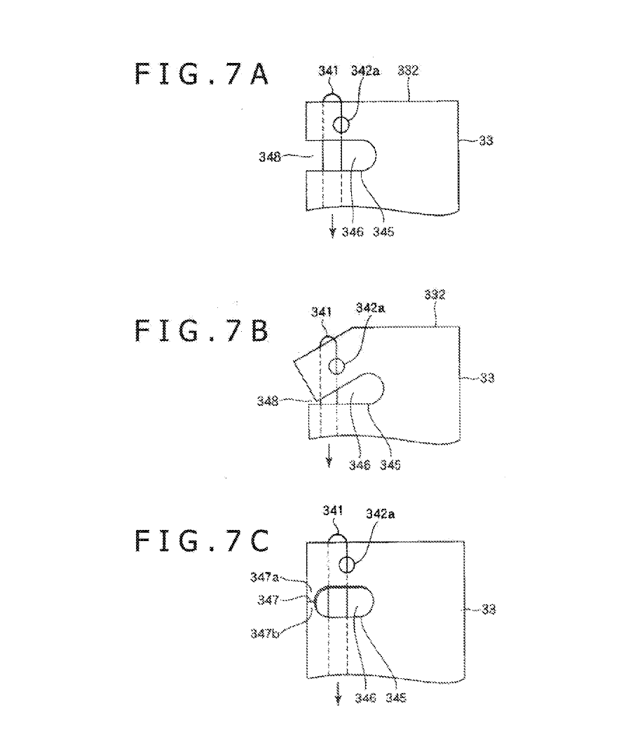

As above-mentioned, in this embodiment, the slit 347 is provided, and the slit 347 is used as a path along which the blade passes, helps prevent the main body 31 from being deformed under a tension on the string 341. Specifically, for example, as shown in FIG. 7A, the path along which the blade is passed may be composed of a hole 348 instead of the slit 347. In this case, however, depending on the hardness of the main body 31 there may arise a situation where as shown in FIG. 7B, the hole 348 may be crushed through buckling under the tension on the string 341, resulting in deformation of the main body 31. In the case of the slit 347, since parts 347a and 347b on both sides of the slit 347 abut on and are pressed against each other, as shown in FIG. 7C, such a deformation as above-mentioned would not occur, so that the main body 31 is prevented from deformation.

In accordance with an exemplary embodiment, as shown in FIG. 5, the main body 31 is provided in a distal portion thereof with a pair of engaging holes 315 and 316 for engagement with the anchor 81. In addition, the main body 31 is provided in a proximal portion thereof with a pair of engaging holes 317 and 318 for engagement with the anchor 82. Out of the four engaging holes, the engaging holes 315 and 317 are provided in the inner circumferential portion A1, whereas the engaging holes 316 and 318 are provided in the outer circumferential portion A2.

As aforementioned, the main body 31 is flat shaped and is less likely to be crushed in the major axis direction, so that the spacing between the inner circumferential portion A1 and the outer circumferential portion A2 is unlikely to vary. In addition, the inner circumferential portion A1 and the outer circumferential portion A3 are larger in curvature and, hence, less susceptible to deformation, as compared with the front surface A3 and the back surface A4. With the engaging holes 315 and 317 provided in the inner circumferential portion A1 and with the engaging holes 316 and 318 provided in the outer circumferential portion A2, therefore, the engagement between the anchors 81 and 82 and the main body 31 is unlikely to be released.

In addition, the spacing between the engaging holes 315 and 316 and the central portion S4 and the spacing between the engaging holes 317 and 318 and the central portion S4 are approximately equal, which helps ensure that the anchors 81 and 82 serve as markers, whereby the position of the central portion S4 of the main body 31 inside a living body can be easily grasped.

In addition, as shown in FIGS. 4A to 6C, a side surface of the main body 31 can be formed with a plurality of through-holes (side holes) 311 communicating with an internal cavity of the main body 31. Specifically, for example, each of the through-holes 311 connects an outer circumferential surface and an inner circumferential surface of the main body 31, and an end portion of each through-hole 311 communicates with the outer circumferential surface of the main body 31. The internal cavity and the through-holes 311 of the main body 31 function as flow paths through which a liquid flows in a state where the puncture member 3 (medical tube assembly 10) punctures a living body. Specifically, for example, when a living body is punctured with the puncture member 3, if a blood vessel is punctured by mistake, blood flowing out via a wound of the blood vessel flows through the through-holes 311 into the internal cavity of the main body 31, and flows through the internal cavity; thus, flash-back of the blood occurs. By visually confirming the flash-back of the blood, the operator can grasp that he/she has punctured a blood vessel by mistake. In addition, where physiological salt solution is caused to flow into a bladder, it can be confirmed whether the bladder or the urethra has been punctured by mistake, based on the presence or absence of flash-back of the physiological salt solution. Note that the number of the through-holes 311 is not limited to a plurality but may be one.

In accordance with an exemplary embodiment, the through-holes 311 are formed in a central portion of the main body 31, specifically, for example at a proximal portion of the distal separable piece 32 and a distal portion of the proximal separable piece 33. When the implant main body 91 is placed indwelling in a living body, the central portion of the main body 31 is located between a urethra 1300 and a vagina 1400 (see FIGS. 22A and 22B). With the through-holes 311 formed in the central portion of the main body 31, therefore, puncturing of the urethra 1300 by the puncture member 3 by mistake can be confirmed with enhanced reliability.

Note that the region where the through-holes 311 are formed is not restricted to the central portion of the main body 31, but may be, for example, the whole body of the main body 31.

In addition, the layout of the through-holes 311 is not specifically limited, but may be appropriately set according to various conditions. In this embodiment, the through-holes 311 are laid out regularly, specifically, for example, at regular intervals in the axial direction of the main body 31 and at regular intervals in the circumferential direction of the main body 31. In addition, each of dimensions such as diameter and pitch of the through-holes 311 are not particularly limited but may be appropriately set according to various conditions. Note that other layouts of the through-holes 311 include spiral layout and irregular layouts.

The shape of the through-holes 311 is not specifically restricted. In this embodiment, the through-holes 311 are circular on a plan view basis. Note that other shapes of the through-holes 311 on a plan view basis include ellipses, and polygons such as tetragons.

The main body 31 as above is provided at the distal end thereof with the needle body 35. As depicted in FIG. 5, the needle body 35 can include a needle tip 351, which is tapered off, and a proximal section 352 provided on the proximal side of the needle tip 351. The proximal section 352 is inserted in the main body 31, whereby the needle body 35 is detachably retained on the main body 31. Note that the proximal section 352 is fitted in the main body 31 with such a force that the needle body 35 can be prevented from being unintentionally detached from the main body 31. Note that the needle body 35 may be configured to be integral with the main body 31.

The proximal section 352 is provided with an engaging section 353 for engagement with the distal portion 711 of the insertion section 71. The engaging section 353 can include a recess, and, in an inserted state where the insertion section 71 is inserted in the puncture member 3, the distal portion 711 is located inside the engaging section 353. With the engaging section 353 provided, displacement of the needle body 35 relative to the insertion section 71 is restrained, and puncture of a living body by the puncture member 3 can be smoothly performed.

Note that in the case where a cross-sectional shape of at least the distal portion 711 of the insertion section 71 is a flat shape, it can be preferable, for example, that a cross-sectional shape of the engaging section 353 is formed in accordance with a cross-sectional shape of the distal portion 711, in other words, a cross-sectional shape of the engaging section 353 is also a flat shape, which helps ensure that in a state where the engaging section 353 and the distal portion 711 are in engagement with each other, the flat shape of the engaging section 353 and the flat shape of the distal portion 711 overlap each other. Due to this overlapping, rotation of the sheath 30 about the axis thereof relative to the insertion section 71 is restricted.

The puncture member 3 has thus been described above. The center angle θ4 of the puncture member 3 is not particularly limited, and is appropriately set according to various conditions. As will be described later, the center angle θ4 is so set that the needle body 35 can enter a patient's body via an inguinal region on one side of the patient, pass between the urethra and the vagina, and exit the body via an inguinal region on the other side. Specifically, the center angle θ4 is, for example, preferably 150° to 270°, more preferably 170° to 250°, and further preferably 190° to 230°.

The materials constituting the main body 31 and the needle body 35 are preferably rigid materials such as to maintain the shape of the puncture member 3 and the internal space (internal cavity) in a state where the puncture member 3 is inserted in a living body. Examples of such rigid materials applicable here include, for example, various resin materials such as polyethylene, polyimides, polyamides, polyester elastomers, polypropylene, etc. and various metallic materials such as stainless steel, aluminum or aluminum alloys, and titanium or titanium alloys. Note that the main body 31 and the needle body 35 may not necessarily be configured by adopting rigid materials, but may be configured by adopting other materials than rigid materials; in the latter case, the wall may be reinforced with a reinforcement member. For example, a braiding with high strength may be embedded in the wall, whereby the shape and the internal space can be maintained in the state where the puncture member 3 is inserted in a living body. Another example of the reinforcement member is a spiral body, which is embedded in the wall of the main body 31, whereby flexibility can be relatively ensured while the internal space is retained to such an extent that an inserted article can be slid therein.

The main body 31 is preferably light-transmitting so that the inside thereof can be visually checked externally, which helps make it possible, for example, to check whether the distal portion 711 of the insertion section 71 inserted to the inside is in engagement with the engaging section 353, whether the string 341 has not been cut, and so on. In addition, flash-back of blood or the like to be described later can also be visually checked in a relatively easy manner.

The aforementioned puncture member 3 (main body 31) and the insertion section 71 which is inserted into the main body 31 constitute a medical tube assembly 10; use of the puncture apparatus 1 is started with these members being in the state of the medical tube assembly 10.

Note that the number and layout of the holes (342a, 342b and 342c) through which to pass the string 341 are not particularly limited insofar as the connected state of the distal separable piece 32 and the proximal separable piece 33 can be maintained by the string 341. The string 341 may not necessarily be endless, but may have ends, specifically, one end and the other end. For example, a string having ends may be prepared, one end of the string may be passed through the hole 342a and the proximal-side opening 332 to form a loop, and the other end may be passed through the holes 342b and 342c to form a loop. The string 341 can include cords and belts, which can be used similarly to the string 341.

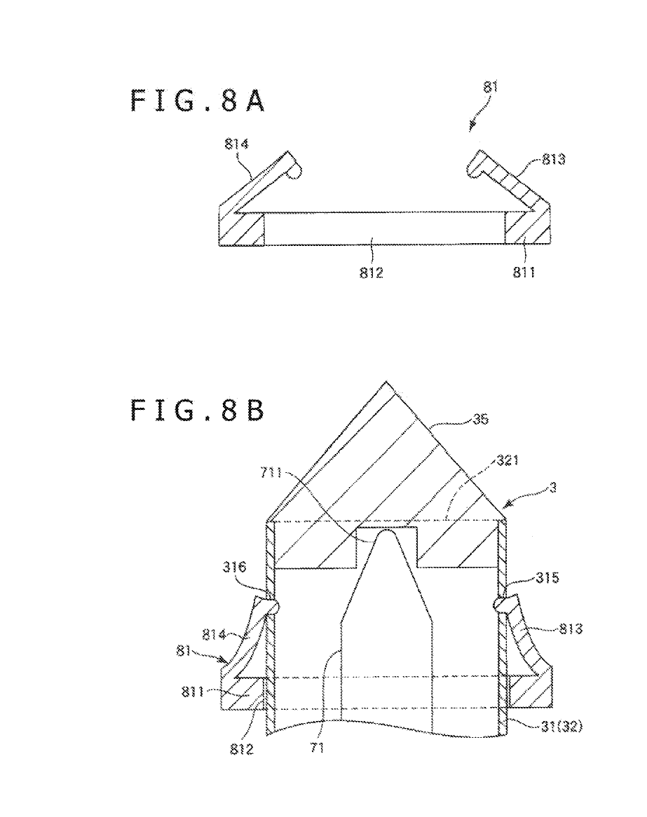

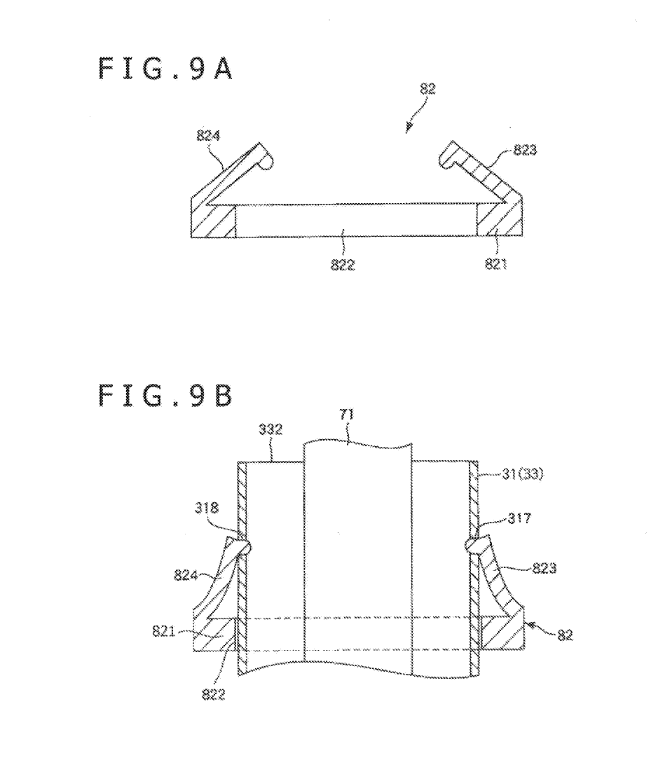

As shown in FIG. 8A, the anchor (second anchor) 81 can include a base section 811 having an insertion hole 812 in and through which the main body 31 is inserted and passed, and a pair of claw sections 813 and 814 projecting from the base section 811 and engaging with the pair of engaging holes 315 and 316. The cross-sectional shape of the insertion hole 812 corresponds to the cross-sectional shape of the main body 31. In a state where the puncture member 3 is inserted in and passed through the insertion hole 812, therefore, rotation of the anchor 81 relative to the puncture member 3 is restrained, and the positional relation between these members is maintained appropriately. When the puncture member 3 is inserted into the insertion hole 812 and the puncture member 3 is pushed forward in relation to the anchor 81, the claw sections 813 and 814 are engaged with the engaging holes 315 and 316, as shown in FIG. 8B. As a result, the anchor 81 is engaged with the distal separable piece 32. In the engaged state, the base section 811 is located on the proximal side as compared with the claw sections 813 and 814. As aforementioned, rotation of the anchor 81 relative to the puncture member 3 is restrained in the state where the puncture member 3 is inserted in and passed through the insertion hole 812, and, accordingly, the engagement between the claw sections 813 and 814 and the engaging holes 315 and 316 can be developed relatively assuredly.

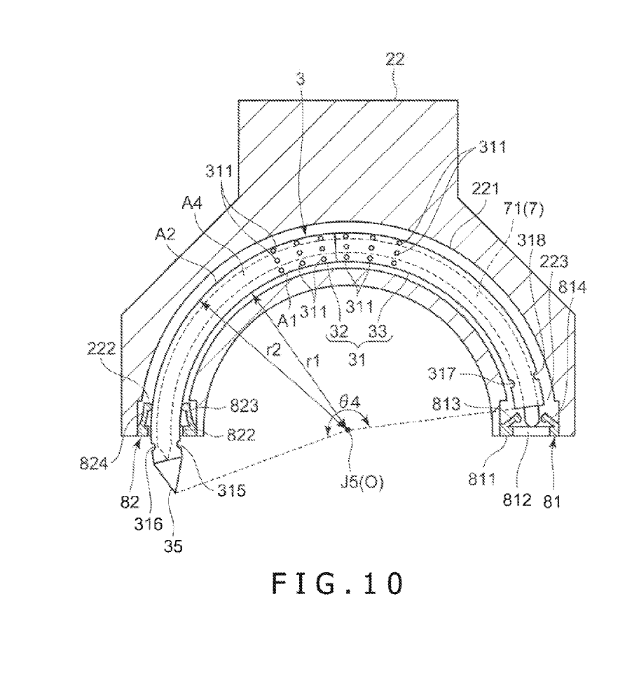

Similarly, as shown in FIG. 9A, the anchor (first anchor) 82 can include a base section 821 having an insertion hole 822 in and through which the main body 31 is inserted and passed, and a pair of claw sections 823 and 824 projecting from the base section 821 and engaging with the pair of engaging holes 317 and 318. The cross-sectional shape of the insertion hole 822 corresponds to the cross-sectional shape of the main body 31. In a state where the puncture member 3 is inserted in and passed through the insertion hole 822, therefore, rotation of the anchor 82 relative to the puncture member 3 is restrained, and the positional relation between these members is maintained appropriately. When the puncture member 3 is inserted into the insertion hole 812 and the puncture member 3 is pushed forward in relation to the anchor 82, therefore, the claw sections 823 and 824 are engaged with the engaging holes 317 and 318, as depicted in FIG. 9B. As a result, the anchor 82 is engaged with the proximal separable piece 33. As aforementioned, rotation of the anchor 82 relative to the puncture member 3 is restrained in the state where the puncture member 3 is inserted in and passed through the insertion hole 822, and, accordingly, engagement between the claw sections 823 and 824 and the engaging holes 317 and 318 can be developed.

The materials constituting the anchors 81 and 82 are not particularly limited; for example, various resin materials can be used.

The frame (restriction unit) 2 retains the operating member 7 with the puncture member 3 mounted thereto so that the operating member 7 is turnable, and fixes the insertion tool 6 and the anchors 81 and 82 in an attachable and detachable manner. The frame 2 has a function of determining a puncture path of the needle body 35 when the puncture member 3 (medical tube assembly 10) punctures the biological tissue.

Specifically, for example, the frame 2 determines the positional relations of the puncture member 3, the urethral-insertion member 4 and the vaginal-insertion member 5 in such a manner that the needle point 351 of the needle body 35 passes the farther side from the center of rotary movement of the puncture member 3 than the urethral-insertion member 4, in other words, in such a manner that the needle body 35 passes between the urethral-insertion member 4 and the vaginal-insertion member 5 without colliding against any of these insertion members when the puncture member 3 punctures the biological tissue.

As shown in FIGS. 1 and 2, the frame 2 can include a bearing section 21 for bearing the shaft section 73 of the operating member 7, a guide section (retaining section) 22 for guiding the puncture member 3 and retaining the second and first anchors 81 and 82 in an attachable and detachable manner, an interlock section 23 interlocking the bearing section 21 and the guide section 22, and a fixing section 24 to which the insertion tool 6 is fixed.

The bearing section 21 is located on the proximal side of the puncture apparatus 1, and extends in a direction substantially orthogonal to the axis J1. The bearing section 21 is formed with a through-hole 211 on the axis J1, and the shaft section 73 is turnably inserted in the through-hole 211. As a result, the operating member 7 is supported on the frame 2 so as to be turnable about the axis J1.

The guide section 22 is located on the distal side of the puncture apparatus 1, and is disposed opposite to the bearing section 21. As shown in FIG. 10, the guide section 22 is formed therein with a roughly C-shaped guide groove 221 for accommodating the puncture member 3 and guiding the puncture member 3. In accordance with an exemplary embodiment, as shown in FIG. 11, in a state of being disposed within the guide groove 221, the puncture member 3 has its back surface A4 located on the distal side and has its front surface A3 located on the proximal side.

In addition, the guide section 22 retains the anchors 81 and 82 in an attachable and detachable manner. The anchor 82 is retained to face the distal-side opening 222 so that the insertion hole 822 and the guide groove 221 are continuous with each other. The anchor 81 is retained to face the proximal-side opening 223 of the guide groove 221 so that the insertion hole 812 and the guide groove 221 are continuous with each other.

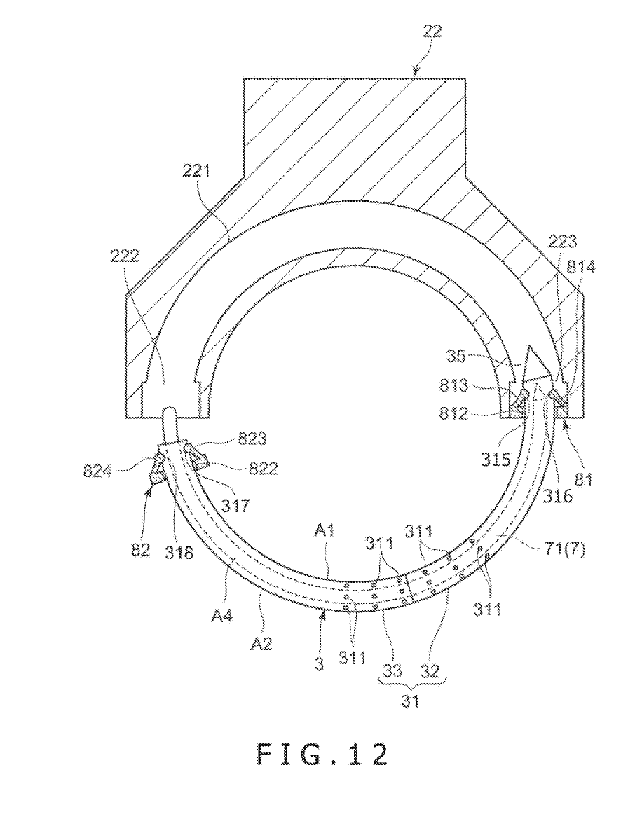

In the initial state, the main body 31 is inserted in and passed through the insertion hole 822 of the anchor 82, and the needle body 35 is protruding from the guide section 22. When the operating member 7 is rotated, the puncture member 3 gradually protrudes from the guide section 22, and, finally, the needle body 35 enters into the guide section 22 via the proximal-side opening 223, as shown in FIG. 12. In this process, on the distal side of the puncture member 3, the puncture member 3 is passed through the insertion hole 812 of the anchor 81, and the claw sections 813 and 814 are engaged with the engaging holes 315 and 316. On the other hand, on the proximal side of the puncture member 3, the claw sections 823 and 824 are engaged with the engaging holes 317 and 318. As a result, the anchors 81 and 82 are engaged with the puncture member 3.

The interlock section 23 interlocks the bearing section 21 and the guide section 22. In accordance with an exemplary embodiment, the interlock section 23 has a rod-like shape extending substantially in parallel to the axis J1. The interlock section 23 functions also as a grip section, and an operator can use the puncture apparatus 1 by gripping the interlock section 23.

The fixing section 24 is disposed opposite to the interlock section 23, with the axis J1 interposed therebetween. The fixing section 24 is provided with a recess 243 in which to fit a support section 60 (described later) of the insertion tool 6, and a male screw 244. With the support section 60 fitted into the recess 243 and with the male screw 244 fastened into a female screw (not illustrated) of the support section 60, the insertion tool 6 can be fixed to the fixing section 24.

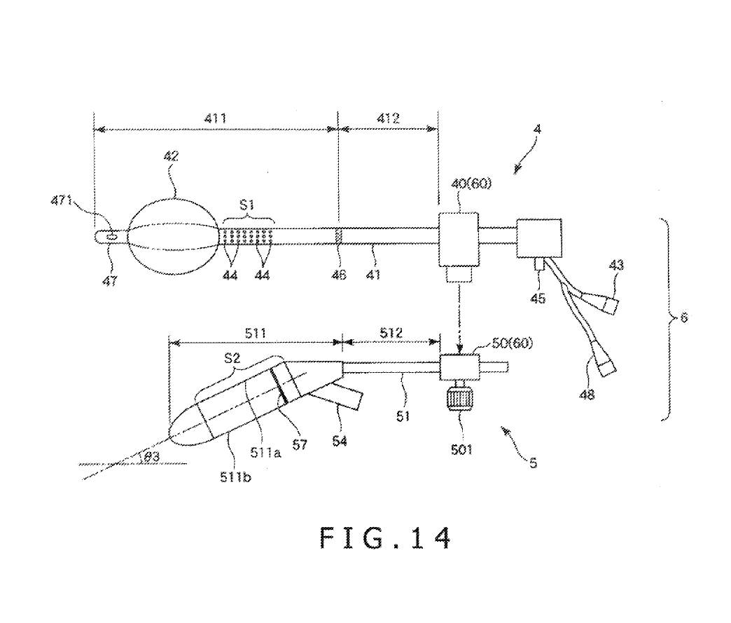

As illustrated in FIGS. 1 and 14, the insertion tool 6 can include a urethral-insertion section (second insertion section) 41 to be inserted into a urethra, a vaginal-insertion section (first insertion section) 51 to be inserted into a vagina, and the support section 60 supporting the urethral-insertion section 41 and the vaginal-insertion section 51. As aforementioned, the insertion tool 6 can include the urethral-insertion member 4 and the vaginal-insertion member 5, wherein the urethral-insertion member 4 has the urethral-insertion section 41, and the vaginal-insertion member 5 has the vaginal-insertion section 51. The support section 60 can include a support section 40 which is possessed by the urethral-insertion member 4 and which supports the urethral-insertion section 41, and a support section 50 which is possessed by the vaginal-insertion member 5 and which supports the vaginal-insertion section 51. In the insertion tool 6, the urethral-insertion member 4 and the vaginal-insertion member 5 can be attached to and detached from each other through the support sections 40 and 50. The urethral-insertion member 4 and the vaginal-insertion member 5 will be sequentially described below.

The urethral-insertion member 4 can include the urethral-insertion section 41 which is elongated and which, from its distal end to its intermediate portion, is to be inserted into the urethra, and the support section 40 supporting the urethral-insertion section 41. Note that in the following, for convenience of explanation, the part located inside the urethra (inclusive of the bladder) in the mounted state will be referred to also as the “insertion section 411,” whereas the part exposed from the urethral orifice to the outside of the body in the mounted state will be referred to also as the “non-insertion section 412.”

The urethral-insertion section 41 has a straight tubular shape with the distal end rounded. The insertion section 411 is provided at a distal portion thereof with an expandable and contractible balloon 42, and a urine drain section 47. The balloon 42 functions as a restricting section, which restricts the position in the axial direction of the urethral-insertion member 4 within the urethra. Specifically, for example, at the time of using the puncture apparatus 1, the balloon 42 is expanded after inserted into a patient's bladder. Then, the expanded balloon 42 is caught on a bladder neck, whereby the position of the urethral-insertion member 4 relative to the bladder and the urethra is fixed. In accordance with an exemplary embodiment, the urine drain section 47 can be used for draining urine present in the bladder.

The balloon 42 is connected to a balloon port 43 provided at a proximal portion of the urethral-insertion section 41, through the inside of the urethral-insertion section 41. A balloon expanding device such as a syringe can be connected to the balloon port 43. The balloon 42 is expanded when a working fluid (a liquid such as physiological saline, or a gas or the like) is supplied from the balloon expanding device into the balloon 42. On the contrary, the balloon 42 is contracted when the working fluid is drawn out of the balloon 42 by the balloon expanding device. Note that in FIG. 14, the contracted state of the balloon 42 is indicated by alternate long and two short dashes line, and the expanded state of the balloon 42 is indicated by solid line.

In accordance with an exemplary embodiment, the urine drain section 47 can be provided with a drain hole 471 through which the inside and the outside of the urine drain section 47 communicate with each other. The urine drain section 47 is connected to a urine drain port 48 provided at a proximal portion of the urethral-insertion section 41, through the inside of the urethral-insertion section 41. Therefore, urine introduced via the drain hole 471 can be drained via the urine drain port 48.

The balloon 42 and the urine drain section 47 can be configured, for example, by a double lumen.

The insertion section 411 is formed at an intermediate portion thereof with a plurality of suction holes 44. The plurality of suction holes 44 are disposed over the whole circumferential range of the urethral-insertion section 41. Each of the suction holes 44 is connected to a suction port 45 provided at a proximal portion of the urethral-insertion section 41, through the urethral-insertion section 41. A suction device such as a pump can be connected to the suction port 45. When the suction device is operated in a state where the urethral-insertion section 41 is inserted in the urethra, a urethral wall can be secured by suction onto the urethral-insertion section 41. When in this condition the urethral-insertion section 41 is pushed in toward the distal side (into the living body), the urethra is also pushed in attendantly, whereby it is possible, for example, to shift the bladder to such a position as not to overlap with a puncture path for the puncture member 3, and thereby to secure the puncture path for the puncture member 3. Accordingly, puncture by the puncture member 3 can be performed relatively accurately and safely. Note that the number of the suction holes 44 is not particularly limited, for example, only one suction hole may be provided. In addition, the layout of the suction holes 44 is not specifically restricted; for example, the suction holes 44 may be formed in only part in the circumferential direction of the urethral-insertion section 41.

At the boundary between the insertion section 411 and the non-insertion section 412, there is provided a marker 46 for confirming the depth of insertion of the urethral-insertion section 41 into the urethra. The marker 46 is located at the urethral orifice when the urethral-insertion section 41 is inserted in the urethra and the balloon 42 is located inside the bladder. As a result, the depth of insertion of the insertion section 411 into the urethra can be easily confirmed. It is sufficient for the marker 46 to be visibly checkable externally; thus, the marker 46 may be configured, for example, as a colored part, a rugged part or the like. Note that graduations indicative of the distance from the distal end of the urethral-insertion section 41 may be provided in place of the marker 46.

The length of the insertion section 411 is not particularly limited, and may be appropriately set according to the length of the patient's urethra, for example, the shape of the patient's bladder. In view of that the length of a female urethra is generally about 30 to 50 mm, it can be preferred, for example, that the length of the insertion section 411 is about 50 to 100 mm.

The length of the non-insertion section 412 (the spacing between the urethral orifice and the support section 40) is not specifically restricted, and is preferably not more than, for example, about 100 mm, more preferably in the range of about 20 to 50 mm By this, the non-insertion section 412 can be made to have a suitable length, and operability is enhanced. If the length of the non-insertion section 412 exceeds the above-mentioned upper limit, there may arise, depending on the configuration of the frame 2 or the like, a situation in which the center of gravity of the puncture apparatus 1 is largely spaced from the patient and, accordingly, the stability of the puncture apparatus 1 in the mounted state is lowered.

The material constituting the urethral-insertion member 4 is not particularly limited. For example, various metallic materials such as stainless steel, aluminum or aluminum alloys, titanium or titanium alloys, etc. and various resin materials can be used.

Here, the inclination angle θ2 of the plane f9 (plane f1) against the plane f2 orthogonal to the axis J2 of the urethral-insertion section 41 is preferably, for example, about 20° to 60°, more preferably about 30° to 45°, and further preferably about 35° to 40°. In other words, the main body 31 is preferably so set indwelling in a living body that the angle formed between the plane f9 and the plane orthogonal to the axis of the urethra is, for example, about 20° to 60°, more preferably so set indwelling in the living body that the angle is about 30° to 45°, and further preferably so set indwelling in the living body that the angle if about 35° to 40°, which helps ensure that puncture by the puncture member 3 can be performed relatively easily, and the puncture distance in puncture by the puncture member 3 can be made shorter.

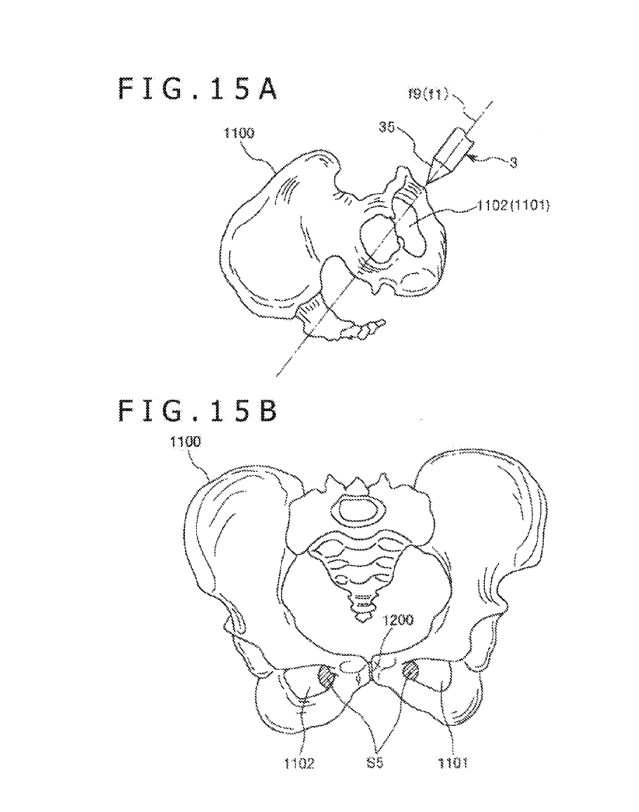

Describing more specifically, with the inclination angle θ2 set within the above-mentioned range, the puncture member 3 can capture left and right obturator foramens 1101 and 1102 of a pelvis 1100 wider on a planar basis, as depicted in FIG. 15A, and a wide puncture space for the puncture member 3 can be secured. In other words, in a state where a patient is set in a predetermined position (lithotomy position), the puncture member 3 can be made to puncture in a direction comparatively nearer to a perpendicular direction relative to the obturator foramens 1101 and 1102. Therefore, the puncture by the puncture member 3 can be carried out relatively easily. In addition, where the puncture member 3 is made to puncture in a direction comparatively nearer to the perpendicular direction relative to the obturator foramens 1101 and 1102, the needle body 35 of the puncture member 3 passes a shallow portion of the tissue, so that the needle body 35 of the puncture member 3 can pass between the left and right obturator foramens 1101 and 1102 while taking a shorter course. Therefore, as shown in FIG. 15B, the puncture member 3 can be made to pass those zones in the obturator foramens 1101 and 1102 which are near a pubic symphysis 1200, preferably, safety zones S5. Since the safety zones S5 are parts where there are few nerves and blood vessels which should be prevented from being damaged, the puncture can be performed by the puncture member 3 relatively safely. Accordingly, a less invasive procedure is realized, and the burden on the patient can be suppressed to a low level. Thus, with the inclination angle θ2 set within the above-mentioned range, the puncture of the patient by the puncture member 3 can be performed more suitably. In addition, the puncture at the aforementioned angle makes it relatively easier to aim at the tissue between a middle-part urethra (which refers to a middle part in the longitudinal direction of the urethra) and the vagina. The position between the middle-part urethra and the vagina is a position suitable as a part where to perform treatment of urinary incontinence by embedding the implant 9.

In accordance with an exemplary embodiment, where the inclination angle θ2 is below the above-mentioned lower limit or above the above-mentioned upper limit, there may arise, depending on individual differences concerning the patient or the posture of the patient during the procedure or the like, a situation where the puncture member 3 cannot capture the obturator foramens 1101 and 1102 wide on a planar basis or where the puncture path cannot be made sufficiently short.

More preferably, the puncture is conducted in a state where the urethra or the vagina or both the urethra and the vagina are positionally shifted in the manner of being pushed in toward the inner side of the body, whereby a region between the middle-part urethra and the vagina can be punctured easily. The method for pushing in either one of the urethra and the vagina toward the inner side of the body can, for example, by a method wherein the urethral-insertion member 4 and/or the vaginal-insertion member 5 is inserted into a suitable position, then, in this condition, the urethra and/or the vagina is attracted by suction by the suction holes 44 and 59 (described later) provided in these insertion members, and thereafter the urethral-insertion member 4 and/or the vaginal-insertion member 5 is moved further toward the inner side of the body along the axis thereof to a predetermined position. Where the puncture is conducted by setting the main body 31 perpendicularly relative to the left and right obturator foramens 1101 and 1102 of the pelvis in the state where at least one of the urethra and the vagina has thus been positionally shifted in the manner of being pushed in toward the inner side of the body, a passage can be formed in a position suitable for indwelling of the implant 9.

In accordance with an exemplary embodiment, It can be preferable to form the passage by adopting a setting such that the trajectory of the main body 31 passes the safety zones S5 in the left and right obturator foramens 1101 and 1102 of the pelvis, shifting at least one of the urethra and the vagina toward the inner side of the body so that the trajectory is positioned between the middle-part urethra and the vagina, and performing the puncture by the main body 31 along the trajectory.

As shown in FIGS. 1 and 14, the vaginal-insertion member 5 can include the vaginal-insertion section (first insertion section) 51 which is elongated and which, from its distal end to its intermediate portion, is to be inserted in the vagina, and the support section 50 which supports the vaginal-insertion section 51. Note that in the following, for convenience of explanation, the part located inside the vagina in the mounted state will be referred to also as the “insertion section 511,” whereas the part which is exposed from the vaginal orifice to the outside of the body in the mounted state and which ranges to the support section 50 will be referred to also as the “non-insertion section 512.”

The insertion section 511 is elongated in shape. The insertion section 511 extends while being inclined against the insertion section 411 so as to be spaced away from the insertion section 411 on the distal side. With the insertion section 511 inclined against the insertion section 411, the positional relation between the insertion sections 411 and 511 can be made closer to the positional relation between the urethra and the vagina, as compared with the case where the insertion section 511 is not inclined. Therefore, in the mounted state, the puncture apparatus 1 can be held onto the patient more stably, and the burden on the patient can be alleviated. The inclination angle θ3 of the insertion section 511 against the insertion angle 411 is not particularly limited; for example, the inclination angle θ3 is preferably about 0° to 45°, more preferably about 0° to 30°. This helps enable the above-mentioned effects to be exhibited more remarkably. In accordance with an exemplary embodiment, if the inclination angle θ3 is below the above-mentioned lower limit or above the above-mentioned upper limit, there may arise, depending on individual differences concerning the patient or the posture of the patient during the procedure, a situation in which the vagina or the urethra is unnaturally deformed in the mounted state, and the puncture apparatus 1 is not stably held.

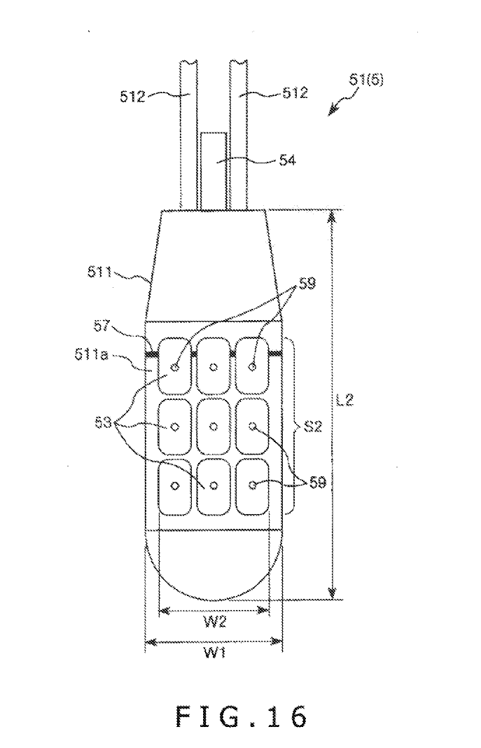

As shown in FIG. 16, the insertion section 511 has a flat shape crushed in the vertical direction of the puncture apparatus 1 (in the direction in which the urethra and the vagina are arrayed). In accordance with an exemplary embodiment, the insertion section 511 can include a central portion which is substantially constant in width, and a distal portion which is somewhat rounded. The length L2 of the insertion section 511 is not particularly limited, and is preferably about 20 to 100 mm, more preferably about 30 to 60 mm. In addition, the width W1 of the insertion section 511 is not specifically restricted, and is preferably, for example, about 10 to 50 mm, more preferably about 20 to 40 mm. Further, the thickness of the insertion section 511 is not particularly limited, and is preferably, for example, about 5 to 25 mm, more preferably about 10 to 20 mm. With the length, width and thickness set in these ranges, the insertion section 511 is made to have a shape and a size suited to general vaginas. Accordingly, the stability of the puncture apparatus 1 in the mounted state is increased, and the burden on the patient is lessened.

An upper surface (a surface on the urethral-insertion section 41 side) 511a of the insertion section 511 is formed with a plurality of bottomed recesses 53. Note that the number of the recesses 53 is not particularly limited, and, for example, only one recess may be provided. At a bottom surface of each recess 53, there is provided a single suction hole 59. Each suction hole 59 is connected to a suction port 54 provided at a proximal portion of the insertion section 511, by way of the inside of the insertion section 511. The suction port 54 is so provided as to be located outside the living body in the mounted state. A suction device such as a pump can be connected to the suction port 54. When the suction device is operated in a state where the insertion section 511 is inserted in the vagina, a vaginal anterior wall which is an upper surface of the vaginal wall is secured by suction onto the insertion section 511. When the vaginal-insertion section 51 is pushed in toward the distal side (into the body) with the vaginal wall thus fixed by suction, the vaginal wall can be pushed in attendantly. Therefore, the disposition and shape of the vaginal wall can be conditioned, a puncture path for the puncture member 3 can be secured, and puncture by the puncture member 3 can be carried out relatively accurately and safely.

The region S2 where the plurality of recesses 53 are formed is disposed opposite to a region S1. The needle tip of the puncture member 3 passes between these regions S1 and S2. Since a urethral posterior wall which is a lower surface of the urethral wall is suction held onto the insertion section 411 in the region S1 as described before and the vaginal anterior wall is suction held onto the insertion section 511 in the region S2, the urethral wall and the vaginal wall are spaced wider apart from each other between the regions S1 and S2. Therefore, by passing the puncture member 3 through such a region, the puncture by the puncture member 3 can be performed safely.

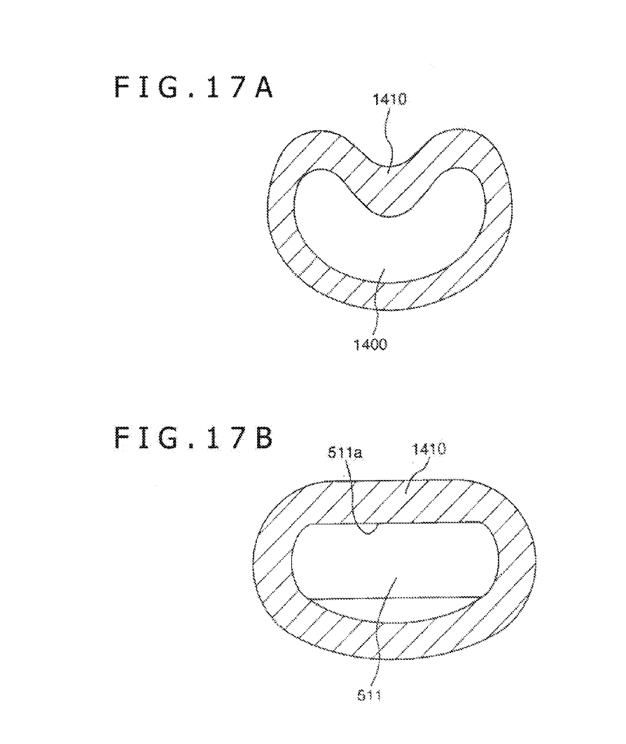

The region S2 stretches over substantially the whole range in the width direction of the upper surface 511a. The width W2 of the region S2 is not particularly limited, and is preferably, for example, about 9 to 39 mm, more preferably about 19 to 29 mm. This enables the vaginal anterior wall to be suction held onto the insertion section 511 reliably, without being considerably influenced by the shape of the vaginal wall. Especially, for example, a patient may have a vagina 1400 shaped as shown in FIG. 17A, wherein part of a vaginal anterior wall 1410 droops down into the inside of the vagina. Even in such a case, setting the width W2 as above-mentioned ensures that as shown in FIG. 17B, not only the drooping-down part but also the parts on both sides of the drooping-down part can be held reliably during suction. Therefore, the vaginal anterior wall can be spaced apart from the urethra reliably, without being influenced by the shape of the vagina. Particularly, in this embodiment, the insertion section 511 is flat shaped, so that the vaginal anterior wall can be suction held in the manner of being spaced farther away from the urethra, and the biological tissue between the urethral wall and the vaginal wall can be widened.

In addition, the insertion section 511 is provided with a marker (puncture position confirmation section) 57 with which the puncture route of the puncture apparatus 1 can be confirmed. In accordance with an exemplary embodiment, the puncture apparatus can be fixed so as to puncture the region between the vaginal wall, which is present on the upper surface of the position where the marker 57 exists, and the urethral wall. As a result, the operability and safety of the insertion tool 6 can be enhanced. The marker 57 is provided at least on a lower surface 511b of the insertion section 511. The lower surface 511b is a surface which is oriented toward the vaginal orifice side and is visible by the operator through the vaginal orifice, in the inserted state. With the marker 57 provided on the lower surface 511b, therefore, the puncture route of the puncture apparatus 1 can be reliably confirmed. In addition, the depth of insertion of the insertion section 511 into the vagina can also be confirmed. Note that it is sufficient for the marker 57 to be externally visible, and the marker 57 can be configured as a colored part or a rugged part.

In accordance with an exemplary embodiment, the non-insertion section 512 is in the shape of a thin bar extending substantially in parallel to the urethral-insertion section 41. The spacing D between the non-insertion section 512 and the urethral-insertion section 41 is not particularly limited, and is, for example, preferably about 5 to 40 mm, correspondingly to the spacing between the urethral orifice and the vaginal orifice in most women.

The length of the non-insertion section 512 (the spacing between the vaginal orifice and the support section 50) is not specifically restricted, and is preferably not more than, for example, about 100 mm, more preferably in the range of about 20 to 50 mm. By this, the non-insertion section 512 can be made to have a suitable length, and its operability can be enhanced. If the length of the non-insertion section 512 exceeds the above-mentioned upper limit, there may arise, depending on the configuration of the frame 2, a situation in which the center of gravity of the puncture apparatus 1 is largely spaced from the patient and, accordingly, the stability of the puncture apparatus 1 in the mounted state is lowered.

The support section 50 is provided with a male screw 501. With the male screw 501 fastened into a female screw (not illustrated) provided in the support section 40, the support sections 40 and 50 can be fixed to each other.

The material constituting the vaginal-insertion member 5 is not specifically restricted. In this case, there can be used, for example, various metallic materials such as stainless steel, aluminum or aluminum alloys, titanium or titanium alloys, and various resin materials, like in the case of the urethral-insertion member 4.

The configuration of the puncture apparatus 1 has thus been described above.

Note that while the urethral-insertion member 4 and the vaginal-insertion member 5 constituting the insertion tool 6 have been configured to be attachable to and detachable from each other in the puncture apparatus 1, this configuration is not restrictive. The urethral-insertion member 4 and the vaginal-insertion member 5 may be so configured that they cannot be attached to or detached from each other.

In addition, while the urethral-insertion section 41 is fixed relative to the support section 40 in the puncture apparatus 1, this configuration is not restrictive. A configuration may be adopted wherein a state where the urethral-insertion section 41 is fixed relative to the support section 40 and a state where the urethral-insertion section 41 is slidable in the axial direction relative to the support section 40 can be selected. Specifically, for example, a configuration may be adopted wherein loosening a screw provided on the support section 40 results in a state where the urethral-insertion section 41 is slidable relative to the support section 40 and wherein fastening the screw results in a state where the urethral-insertion section 41 is fixed relative to the support section 40. According to this configuration, the length of the non-insertion section 412 can be adjusted, so that a user-friendly insertion tool 6 can be is realized. Note that the same applies to the vaginal-insertion section 51.

In accordance with an exemplary embodiment, while the component members are fixed to the frame 2 so that the inclination angle θ2 is constant in the puncture apparatus 1, this configuration is not restrictive, and the inclination angle θ2 may be variable. Where the inclination angle θ2 is variable, the inclination angle θ2 can be adjusted according to the patient, so that a user-friendly puncture apparatus 1 can be realized.

A method of using the puncture apparatus 1 will be described below. Prior to the description of the using method, the implant 9 to be used with the puncture apparatus 1 will be described.

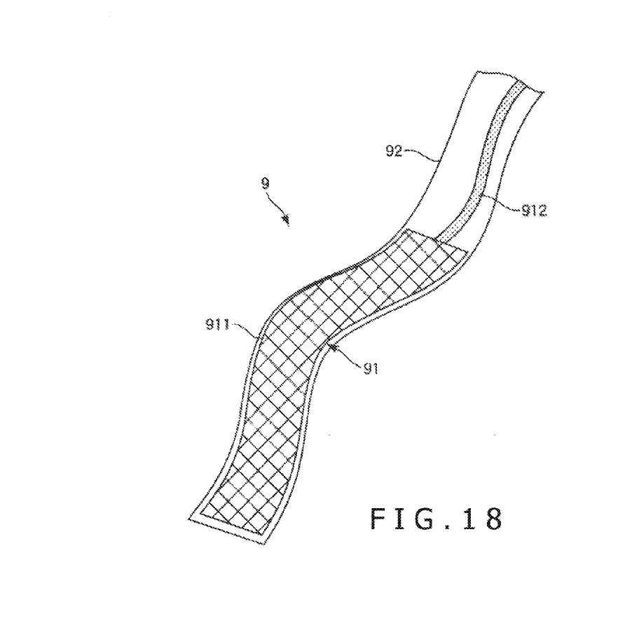

An implant (biological tissue-supporting indwelling article) 9 shown in FIG. 18 is an embeddable instrument for treatment of female urinary incontinence, specifically, an instrument for supporting the urethra. For example, the implant 9 is an instrument which, when the urethra is going to move toward the vaginal wall side, supports the urethra so as to restrict such a movement, in the manner of pulling the urethra in the direction for spacing away from the vaginal wall. As the implant 9, for example, a flexible elongated body can be used.

The implant 9 can include the implant main body (belt-shaped elongated article) 91, and a bag-shaped wrapping material 92 for accommodating the implant main body 91. In addition, the implant main body 91 can include the main body section 911, and a ribbon 912 interlocked to one end of the main body section 911. With the implant 9 provided with the wrapping material 92, contamination of the implant main body 91 can be prevented effectively. Note that a guide wire, a cord, or a string may be used in place of the ribbon 912.

The main body section 911 is net-like in form, and is belt-like in overall shape. Note that the main body section 911 may be composed, for example, of a network-like knitted body knitted by causing linear elements to intersect, specifically, for example, network-formed braiding. Examples of the linear element include those which are circular in cross section, and those which are flat shaped in cross section, namely, belt-shaped (ribbon-shaped) ones.

The materials constituting the main body section 911, the ribbon 912 and the wrapping material 92 are not particularly limited. For example, various resin materials which are biocompatible such as polypropylene, polyesters, nylon, and fibers can be used as the materials.

Note that the implant 9 is not limited to the above-mentioned network-formed one, so long as the same or equivalent effect can be exhibited. The implant 9 and the sheath 30 as above constitute an intrapelvic treatment kit of the present disclosure.

An operating procedure of the puncture apparatus 1, specifically, for example, a procedure hand for embedding the implant 9 into a living body will be described.

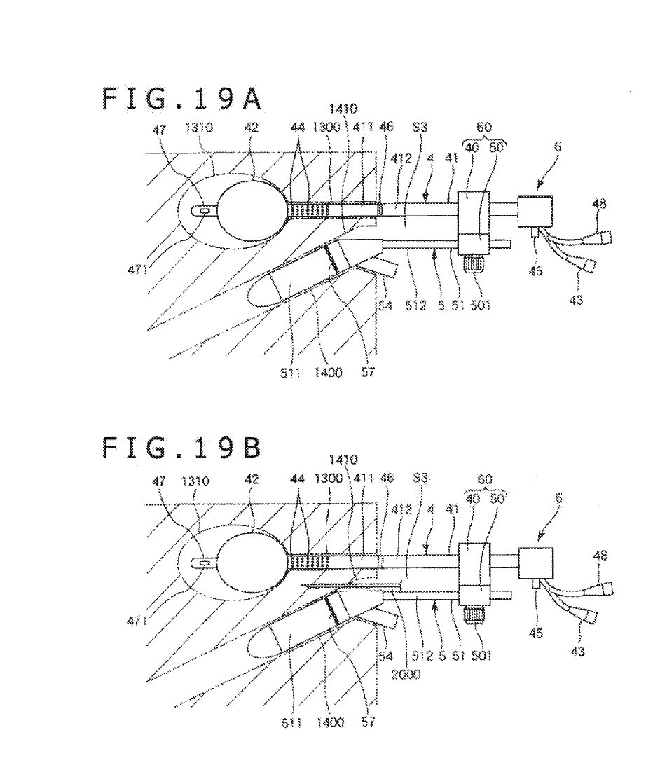

First, a patient is placed in a lithotomy position on an operating table, and the insertion tool 6 is mounted onto the patient, as depicted in FIG. 19A. Specifically, for example, first, the urethral-insertion section 41 of the urethral-insertion member 4 is inserted into the patient's urethra 1300. In this case, the depth of insertion is confirmed with the marker 46, and the balloon 42 is disposed inside the bladder 1310. The urethra 1300 is corrected into a predetermined shape by the urethral-insertion section 41 having the predetermined shape. In the case of this embodiment, the urethra is corrected into a rectilinear shape by the urethral-insertion section 41 which is rectilinear in shape.

Next, the balloon 42 is expanded, and urine is drained from within the bladder 1310 via the drain hole 471, as required. In accordance with an exemplary embodiment, the vaginal-insertion section 51 of the vaginal-insertion member 5 is inserted into the patient's vagina 1400. In this case, the puncture position is confirmed with the marker 57, and insertion into a suitable depth is performed. Then, the support sections 40 and 50 are fixed by operating the male screw 501. By this, the mounting of the insertion tool 6 onto the patient is completed. In this state, the non-insertion sections 412 and 512 are spaced apart from each other, and, further, the support section 60 is spaced apart from a body surface between the urethral orifice and the vaginal orifice, so that the body surface is exposed. In addition, in the case where the insertion section 511 and the vaginal anterior wall are spaced apart from each other to form a gap (space) therebetween, there is formed a space S3 for permitting a syringe to puncture the biological tissue between the urethra and the vagina via the body surface between the urethral orifice and the vaginal orifice.

Subsequently, suction devices are connected to the suction ports 45 and 54, and the suction devices are operated, to suction hold the urethral posterior wall onto the urethral-insertion section 41 and suction hold the vaginal anterior wall onto the vaginal-insertion section 51. For example, when the urethral posterior wall is suction held onto the urethral-insertion section 41 properly, the suction holes 44 are closed with the urethral wall, so that the suction via the suction port 45 is stopped or weakened. Similarly, when the vaginal anterior wall is suction held onto the vaginal-insertion section 51 properly, the suction holes 59 are closed with the vaginal wall, so that the suction via the suction port 54 is stopped or weakened. Therefore, on the basis of the manners of suction via the suction ports 45 and 54 (for example, on the basis of the magnitudes of the sounds generated upon the suction), the operator can check whether or not the urethral posterior wall and the vaginal anterior wall are suction held onto the urethral-insertion section 41 and the vaginal-insertion section 51 properly. Note that the insertion tool 6 may be provided with a checking mechanism for mechanically checking the suction-held state. The checking mechanism is not specifically restricted, so long as the suction-held state can be checked by use of the mechanism. For example, there may be adopted a configuration including a flow rate measuring section (negative pressure meter) for measuring the flow rate through the suction port 54, and a determining section for determining whether or not the suction holding is performed properly, on the basis of the measurement results supplied from the flow rate measuring section.

Next, liquid dissection is conducted. Specifically, for example, as shown in FIG. 19B, a puncture needle of a syringe 2000 is made to puncture the vaginal anterior wall 1410 through the space (space S3) between the insertion section 511 and the vaginal anterior wall 1410, and a liquid such as physiological saline or local anesthetic is injected into the biological tissue in a region between the urethra 1300 and the vagina 1400 (a region between the region S1 and the region S2). As a result, the biological tissue between the regions S1 and S2 is expanded, the urethral posterior wall is pressed against the urethral-insertion section 41, and the vaginal anterior wall 1410 is pressed against the vaginal-insertion section 51.

Here, it is preferable to continue the suction via the suction holes 44 and 59 even during the liquid dissection. When the urethral posterior wall is pressed against the urethral-insertion section 41 by the liquid dissection, the urethral posterior wall is suction held onto the urethral-insertion section 41 more securely, so that the suction through the suction port 45 is stopped or weakened. Similarly, when the vaginal anterior wall is pressed against the vaginal-insertion section 51, the vaginal anterior wall is suction held onto the vaginal-insertion section 51 more securely, so that the suction through the suction port 45 is stopped or weakened. Therefore, based on the manners of suction via the suction ports 45 and 54, the operator can check whether or not the liquid dissection is performed properly.

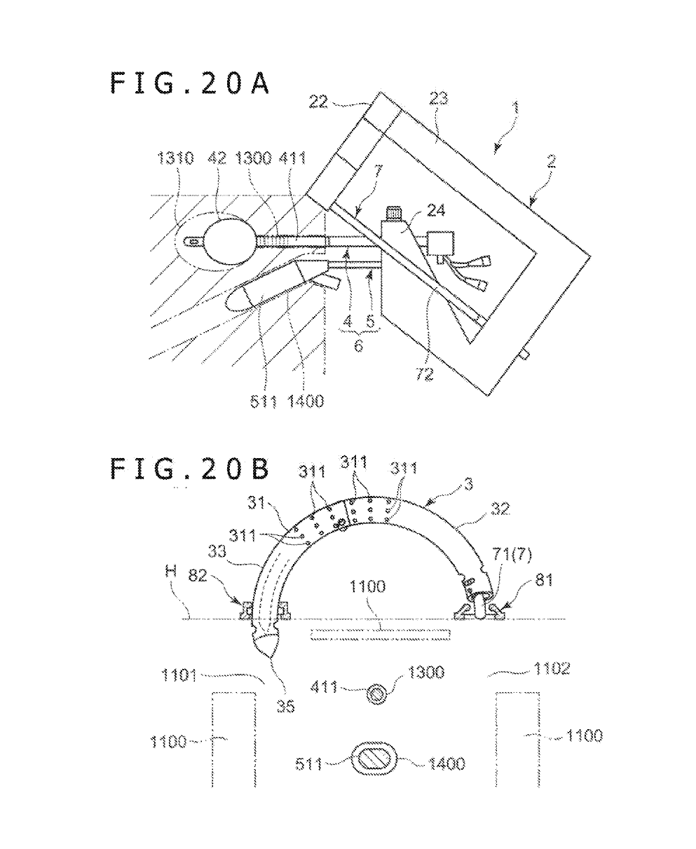

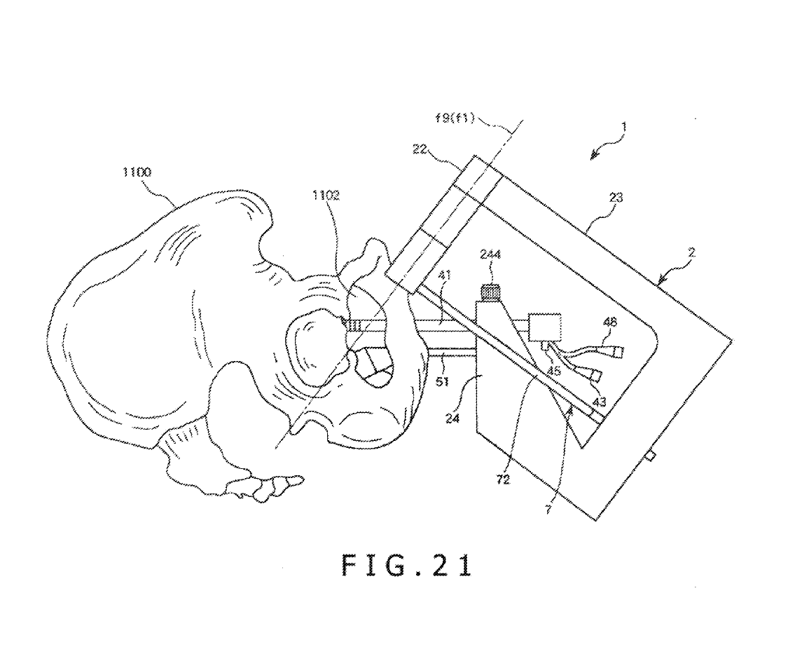

After the liquid dissection is performed and the urethral posterior wall and the vaginal anterior wall are sufficiently spaced apart, the frame 2 can be fixed to the insertion tool 6, as shown in FIGS. 20A and 20B. This results in a state in which the puncture apparatus 1 is mounted onto the patient. In this state, the positional relation between the pelvis 1100 and the puncture apparatus 1 is as depicted in FIG. 21.

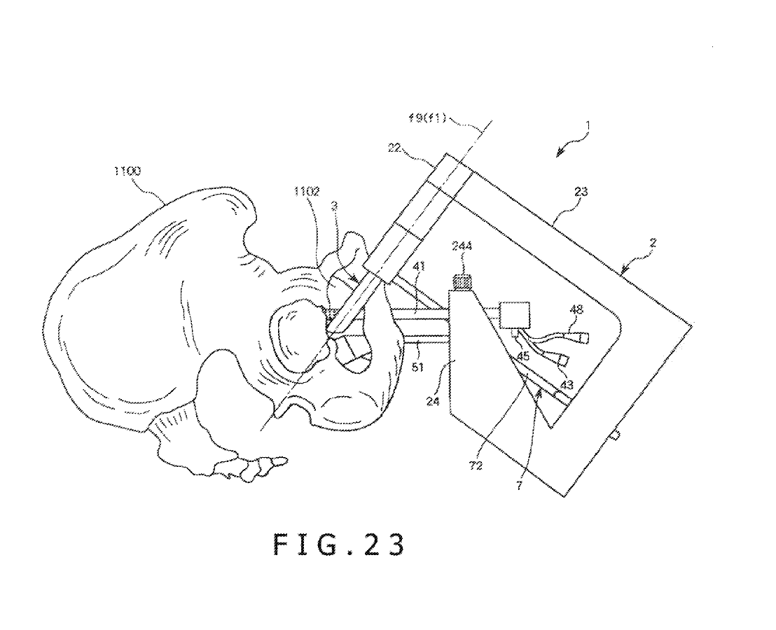

Subsequently, for example, while gripping the interlock section 23 of the frame 2 by one hand, the interlock section 72 of the operating member 7 is grasped by the other hand, and, as shown in FIG. 22A, the operating member 7 is rotated counterclockwise, which causes the needle body 35 of the puncture member 3 to puncture a body surface H at a part (first part) in an inguinal region on the right side of the patient or near the inguinal region, thereby entering the body, to sequentially pass an obturator foramen 1101 on one side, between the urethra 1300 and the vagina 1400, and an obturator foramen 1102 on the other side, then to exit the body via the body surface H at a part (second part) in an inguinal region on the left side or near this inguinal region, and finally to evacuate into the guide section 22 (see FIG. 23).

As a result, the puncture member 3 is disposed in the living body, and, by the aforementioned principle, the anchors 81 and 82 are engaged with the main body 31. Therefore, the anchor 82 abuts on the body surface H, whereby further insertion of a proximal portion of the main body 31 into the living body is restrained. In other words, the state where the proximal end of the main body 31 is exposed outside of the living body can be secured.

Next, when the living body is punctured by the puncture member 3 (medical tube assembly 10), it is checked whether or not a part not to be injured, such as, for example, a blood vessel, the bladder 1310 and the urethra 1300, is punctured and injured by the puncture member 3.

First, in the case where a blood vessel is injured, blood flows through the through-holes 311 into the internal cavity of the main body 31, and flows through the internal cavity. By visually confirming the flash-back of the blood, the operator can grasp that he/she has injured the blood vessel.

In the case of checking whether or not the bladder 1310 or the urethra 1300 is injured, for example, the urine drain port 48 of the urethral-insertion member 4 can be used to cause physiological salt solution to flow into the bladder 1310, and an abdominal pressure is applied. If the bladder 1310 or the urethra 1300 has been injured, the physiological salt solution flows through the through-holes 311 into the internal cavity of the main body 31, and flows through the internal cavity. By visually confirming the flash-back of the physiological salt solution, the operator can grasp that he/she has injured the bladder 1310 or urethra 1300.

Note that in the case where the blood vessel, the bladder 1310, or the urethra 1300 has been injured, a predetermined treatment is performed. Where such injuring has not occurred, the subsequent step is started. In this manner, the procedure can be carried out relatively safely.

Next, the operating member 7 is rotated clockwise in FIG. 22A. In this case, although the puncture member 3 also tends to rotate counterclockwise together with the operating member 7, the abutment of the anchor 81 against the body surface H prevents further rotation (movement) of the puncture member 3. Therefore, while the state where the distal end of the main body 31 is exposed outside of the living body is maintained, the insertion section 71 is drawn out of the puncture member 3 and the living body. Subsequently, the puncture apparatus 1 (other members than the puncture member 3) is dismounted from the patient, and, further, the needle body 35 is detached from the main body 31. This results in a state in which only the main body 31 is disposed inside the living body, as shown in FIG. 22B. The main body 31 is disposed inside the living body, with both the distal-side opening (distal portion) and the proximal-side opening (proximal portion) exposed outside of the living body.

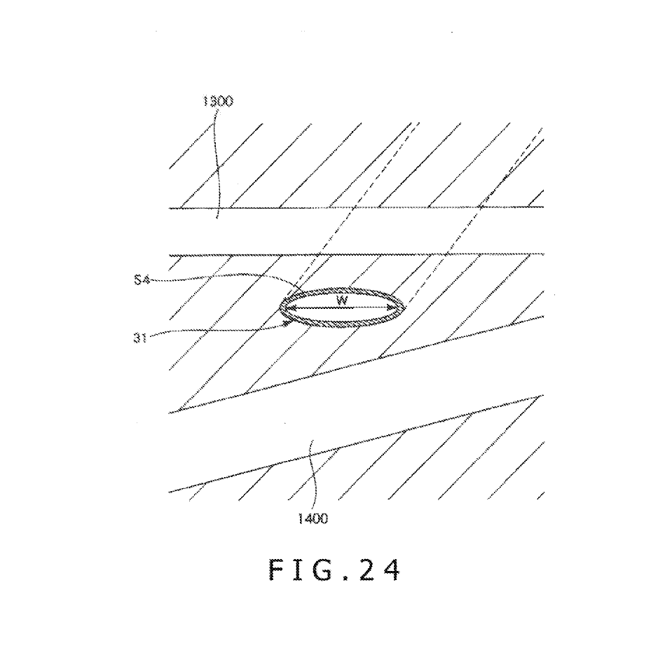

Subsequently, the position of the main body 31 is adjusted, as required. Specifically, for example, the main body 31 is shifted toward the proximal side or the distal side so that the positions of the anchors 81 and 82 relative to the living body will be in left-right symmetry. By this, the central portion S4 of the main body 31 can be reliably positioned between the urethra 1300 and the vagina 1400. In this state, as shown in FIG. 24, the central portion S4 is so disposed that its width direction (the direction of the major axis J32) W is substantially parallel to the urethra 1300. Specifically, the urethra 1300 corrected in shape by the insertion of the urethral-insertion member 4 therein and the width direction W of the central portion S4 are substantially parallel to each other.

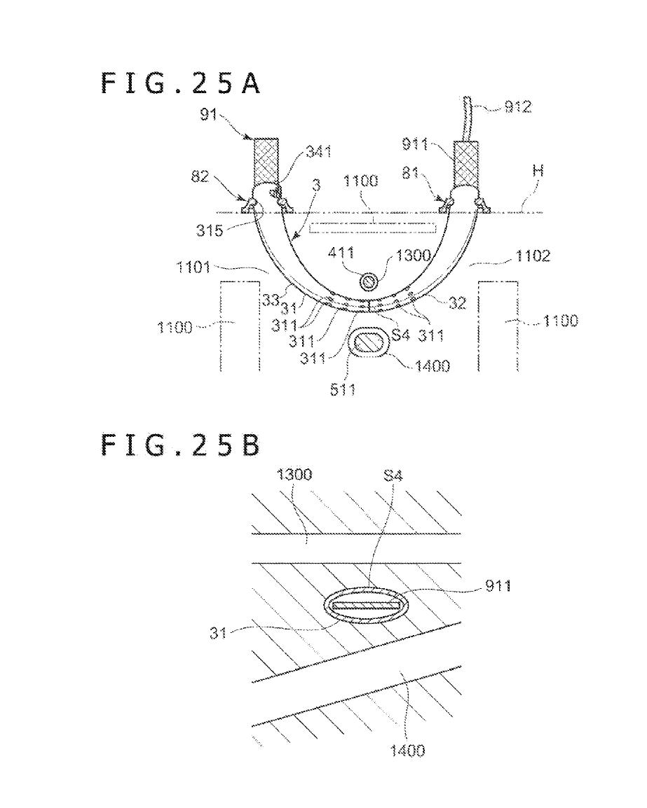

Next, while taking the implant main body 91 out of the wrapping material 92, the implant main body 91 is inserted into the main body 31, and a state where the ribbon 912 is protruded from the proximal-side opening and the distal-side opening of the main body 31 is established, as shown in FIG. 25A. Thus, the implant main body 91 is kept accommodated inside the wrapping material 92 until immediately before disposed inside the main body 31, whereby contamination of the implant main body 91 can be prevented. Note that, as described above, since the main body 31 is flat shaped, the posture of the main body section 911 follows this flat shape. Specifically, for example, as shown in FIG. 25B, the main body section 911 is disposed inside the main body 31 in such a manner that its width direction coincides with the width direction of the main body 31. As for the relation with the urethra 1300, the implant main body 91 is disposed in parallel to the urethra 1300, which has been corrected in shape.

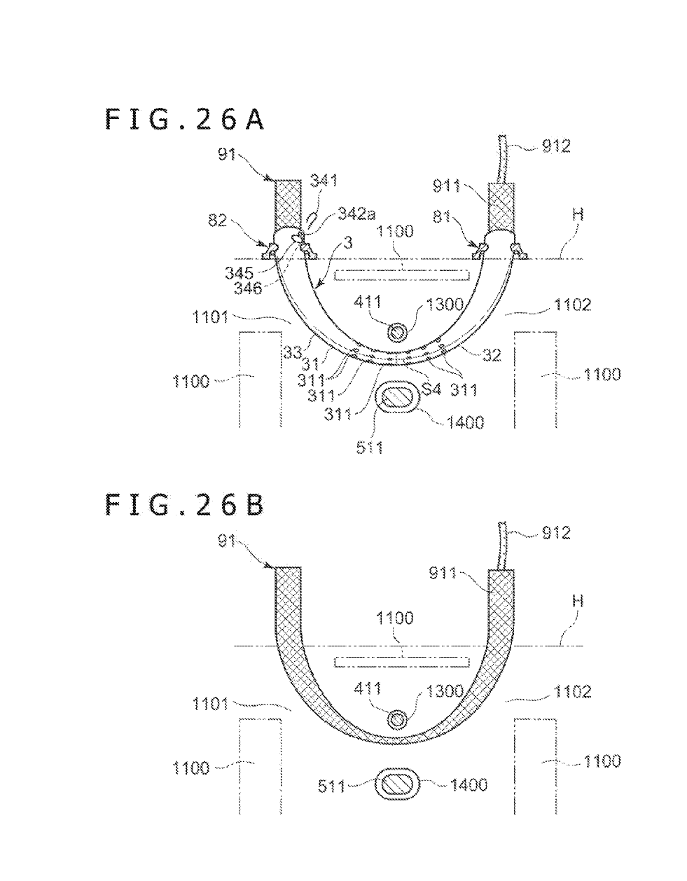

Subsequently, as shown in FIG. 26A, the string 341 exposed from the exposure holes 345 and 346 is cut. This results in a state in which the main body 31 can be separated into the distal separable piece 32 and the proximal separable piece 33. Note that the exposure holes 345 and 346 are located on the proximal side as compared with the anchor 82 and, therefore, can be assuredly exposed outside of the living body. Accordingly, the cutting of the string 341 can be carried out relatively easily.

Next, the suction holding of the urethral posterior wall by the urethral-insertion section 41 and the suction holding of the vaginal anterior wall 1410 by the vaginal-insertion section 51 are stopped. As a result, the positions and shapes of the urethra 1300 and the vagina 1400 are returned into the original natural states.

Subsequently, the connection between the distal separable piece 32 and the proximal separable piece 33 is released, the distal separable piece 32 is drawn out of the living body toward the distal side, and the proximal separable piece 33 is drawn out of the living body toward the proximal side. In this case, the distal separable piece 32 and the proximal separable piece 33 are substantially simultaneously moved in opposite directions, and the distal separable piece 32 and the proximal separable piece 33 are moved in circular arc courses along their shapes, respectively. By this, the main body 31 is smoothly removed out of the living body. As the distal separable piece 32 and the proximal separable piece 33 are gradually removed out of the living body as aforementioned, the surrounding tissue having been pushed open by the main body 31 returns into its original position, and the tissue comes into contact with the implant main body 91 gradually from a central portion toward both end portions of the implant main body 91. As aforementioned, the distal separable piece 32 and the proximal separable piece 33 are moved in the directions along their shapes, and the main body 31 is provided with the internal space in which the implant main body 91 can be moved with sufficiently low friction. This helps enable the implant main body 91 to be left indwelling as it is, without any unnecessary tension exerted thereon. As a result, it is unnecessary to adjust a tension on the implant main body 91. The above operations result in a state in which the implant main body 91 can be embedded in the living body, as shown in FIG. 26B.

In the state where the implant main body 91 is embedded inside the living body, the main body section 911 is disposed substantially in parallel to the urethra 1300, in a region between the urethra 1300 and the vagina 1400. Therefore, the urethra 1300 can be supported in a wider area by the implant main body 91.

Thus, by removing the main body 31 out of the living body through dividing the main body 31, the main body 31 can be easily drawn out of the living body. In addition, since the main body 31 can be drawn out of the living body without need to remove the anchors 81 and 82 from the main body 31, the main body 31 can be drawn out relatively easily. In accordance with an exemplary embodiment, according to such a drawing-out method, the separable pieces 32 and 33 being drawn out exert little influence on the posture of the main body section 911 in the region between the urethra 1300 and the vagina 1400.

In addition, since the separable pieces 32 and 33 are drawn out of the living body in the state where the urethral-insertion member 4 is inserted in the urethra 1300, it is possible to prevent an excessive tension from being exerted on the urethra 1300 by the implant main body 91 placed indwelling in the living body.

Next, the urethral-insertion member 4 is drawn out of the urethra 1300, and the vaginal-insertion member 5 is drawn out of the vagina 1400. After the urethral-insertion member 4 is drawn out, the urethra 1300 returns into its shape in the natural state. Since the main body section 911 is embedded in the tissue, however, a state in which the urethra 1300 in the natural state and the main body section 911 are parallel can be maintained.

Thereafter, unnecessary portions of the implant main body 91 can be cut away, to finish the procedure.

As has been described above, according to the puncture apparatus 1, at the time of placing the implant main body 91 indwelling in a living body, the necessary operation can be dealt with by only low-invasive operations such as puncture with the puncture member 3, without the need to perform a highly invasive incision. Therefore, the burden on the patient is relatively light, and the safety of the patient is relatively high. In addition, the implant main body 91 can be placed indwelling in the living body relatively easily and reliably.

In addition, the living body can be punctured by the puncture member 3 while avoiding the urethra 1300 and the vagina 1400. Therefore, puncturing the urethra 1300 or the vagina 1400 by the puncture member 3 by mistake can be prevented from occurring, and, therefore, safety can be relatively ensured.

In addition, if a blood vessel, the bladder 1310 or the urethra 1300 has been punctured by the puncture member 3 by mistake, the puncture by mistake can be confirmed through flash-back of blood. Accordingly, the procedure can be carried out with enhanced safety.

Further, unlike in the case of conventional incision of the vagina 1400, it is possible to eliminate the possibility of occurrence of a situation in which the implant main body 91 would be exposed to the inside of the vagina 1400 via a wound caused by the incision, or a situation in which complications would be generated such as infection from the wound. Thus, very high safety can be ensured, and the implant main body 91 can be reliably embedded.

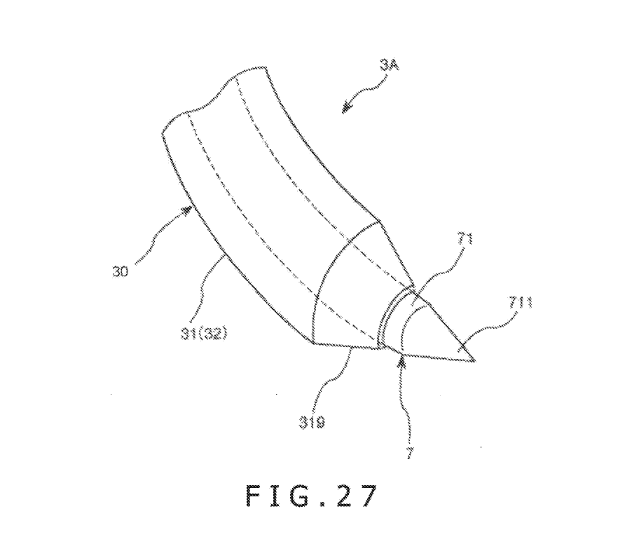

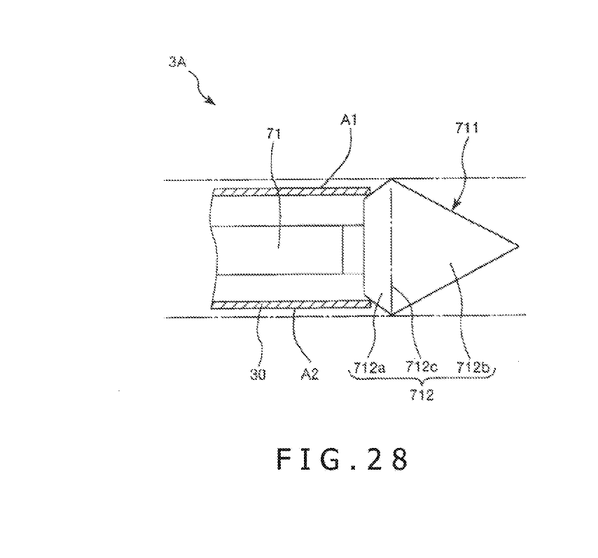

FIG. 27 is a perspective view showing a medical tube assembly according to a second embodiment of the present disclosure. FIG. 28 is a sectional view showing a modification of the medical tube assembly shown in FIG. 27.

Referring to the figures, the second embodiment of a medical tube assembly will be described below. The following description will center on differences from the first embodiment, and descriptions of the same items as above will be omitted.

This embodiment is the same as the aforementioned first embodiment, except mainly for differences in the configuration of puncture member.

As shown in FIG. 27, a puncture member 3A of a medical tube assembly 10 in this embodiment can include a sheath 30 and a distal portion 711 that is a distal portion of an insertion section 71. In other words, the puncture member 3A has a configuration wherein the needle body 35 in the puncture member 3 in the aforementioned first embodiment is changed to the distal portion 711 that is a distal portion of the insertion section 71. In addition, in a state (initial state) where an insertion section 71 is inserted in the puncture member 3, a distal portion 711 as a distal portion of the insertion section 71 is protruding from a distal-side opening of a main body 31. The distal portion 711 protruding from the main body 31 functions also as a needle tip of the puncture member 3A. With the distal portion 711 of the insertion section 71 thus functioning also as the needle body of the puncture member 3A, it is possible to contrive a reduction in the number of members, as compared with the aforementioned first embodiment, for example. In addition, when the puncture member 3 is made to puncture a living body and the insertion section 71 is drawn out of the puncture member 3, the distal-side opening of the main body 31 can be opened. In other words, unlike in the aforementioned first embodiment, in this embodiment it is unnecessary to detach the needle body 35 in order to open the distal-side opening of the main body 31, and, accordingly, the operation can be carried out more smoothly. In addition, the outside diameter of the insertion section 71 and the inside diameter of the distal-side opening of the main body 31 are set to be substantially the same, so that slippage of the insertion section 71 relative to the main body 31 is prevented and, hence, operability is enhanced.

In accordance with an exemplary embodiment, the main body 31 can be provided at its distal portion with a tapered section 319 where its outside diameter gradually increases along the proximal direction from its distal-side opening. The tapered section 319 can function as a dissecting section, which, as the distal portion 711 of the insertion section 71 punctures a living body, dissects the living body in the manner of gradually expanding the living body, following the distal portion 711.

Note that while the taper angle of the tapered section 319 and the taper angle of the distal portion 711 may be the same, they are preferably different from each other as shown in FIG. 27. In this case, it is preferable that the taper angle of the tapered section 319 is smaller than the taper angle of the distal portion 711. This configuration helps enable a smooth puncture.

According to the second embodiment as above, also, the same or equivalent effects to those of the aforementioned first embodiment can be produced.

In addition, as a modification of this embodiment, the following configuration may be mentioned. As shown in FIG. 28, a puncture member 3A is composed of a sheath 30 and a distal portion 711 that is a distal portion of an insertion section 71. In other words, the puncture member 3A has a configuration wherein the needle body 35 in the puncture member 3 in the aforementioned first embodiment is changed to the distal portion 711 of the insertion section 71. In addition, in a state (initial state) where an insertion section 71 is inserted in the puncture member 3, a distal portion 711 as a distal portion of the insertion section 71 is protruding from a distal-side opening of a main body 31.

The distal portion 711 is provided in a detachable manner in relation to the insertion section 71, through screw engagement or fitting. In addition, the distal portion 711 has a needle tip 712 protruding from the distal end of the sheath 30. The needle tip 712 has a flat shape modeled after the sheath 30. In addition, the needle tip 712 can include a gradually increasing area section 712a where its cross-sectional area gradually increases toward its distal end, and a gradually decreasing area section 712b which is provided on the distal side of the gradually increasing area section 712a and in which its cross-sectional area gradually decreases toward its distal end. The minor axis of a boundary 712c between the gradually increasing area section 712a and the gradually decreasing area section 712b can be longer than the minor axis at the distal end of the sheath 30, and the major axis of the boundary 712c can be longer than the major axis at the distal end of the sheath 30, which helps ensure that the inside of a living body can be punctured substantially by only the needle tip 712. Therefore, puncture resistance can be reduced, and a living body can be punctured more smoothly. Note that the minor axis of the boundary 712c may be equal to the minor axis at the distal end of the sheath 30, and the major axis of the boundary 712c may be equal to the major axis at the distal end of the sheath 30.

Note that the second embodiment and the modification thereof are applicable also to each of the embodiments that will be described later.

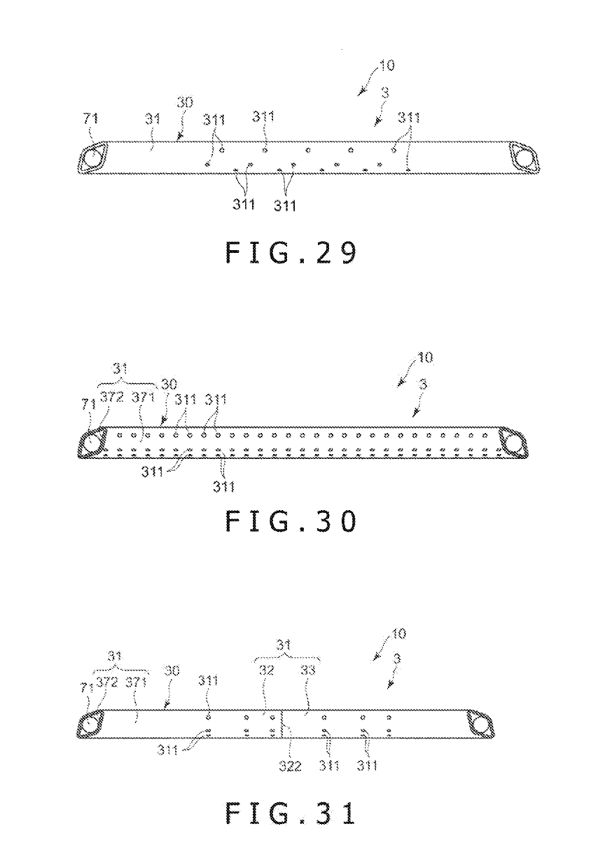

FIG. 29 is a plan view showing a medical tube assembly according to a third embodiment of the present disclosure. Note that in FIG. 29, for convenience of explanation, a puncture member extending in a circular arc shape is depicted in the state of being stretched rectilinearly; in addition, a needle body, a state maintaining mechanism, a distal portion of an insertion section and the like are omitted from the drawing.

Referring to this figure, the third embodiment of a medical tube assembly will be described below. The following description will center on differences from the aforementioned first embodiment, and descriptions of the same items as above will be omitted.

As illustrated in FIG. 29, in a medical tube assembly 10 in this embodiment, a main body 31 of a sheath 30 is like a single tube in shape. In this case, the state maintaining mechanism 34 is omitted. In accordance with an exemplary embodiment, through-holes 311 are laid out spirally.

In this medical tube assembly 10, in placing an implant main body 91 indwelling in a living body, specifically, for example, in drawing out the sheath 30, the sheath 30 is drawn out of the living body toward the distal side or the proximal side.

According to the third embodiment as above, also, the same or equivalent effects to those of the aforementioned first embodiment can be produced.

FIG. 30 is a plan view showing a medical tube assembly according to a fourth embodiment of the present disclosure. Note that in FIG. 30, for convenience of explanation, a puncture member extending in a circular arc shape is depicted in the state of being stretched rectilinearly; in addition, a needle body, a state maintaining mechanism, a distal portion of an insertion section and the like are omitted from the drawing.

Referring to this figure, the fourth embodiment of a medical tube assembly will be described below. The following description will center on differences from the aforementioned third embodiment, and descriptions of the same items as above will be omitted.

As shown in FIG. 30, in a medical tube assembly 10 in this embodiment, a main body 31 of a sheath 30 can include an outer tube 371, and an inner tube 372 disposed on a radially inner side of the outer tube 371. A space between the outer tube 371 and the inner tube 372 and a plurality of through-holes 311 to be described later function as flow paths through which a liquid flows in a state where a puncture member 3 (medical tube assembly 10) is puncturing a living body.

A side surface of the outer tube 371 is formed with the plurality of through-holes 311 communicating with the space between the outer tube 371 and the inner tube 372. In other words, each of the through-holes 311 connects an outer circumferential surface and an inner circumferential surface of the outer tube 371. The through-holes 311 are formed over the whole region, or whole length, of the main body 31, which helps enable the detection of puncture of a blood vessel by mistake to be achieved in a wide range.

Note that an internal cavity of the inner tube 372 is an internal cavity of the main body 31, and an implant main body 91 is disposed in the internal cavity at the time of placing the implant main body 91 indwelling in a living body.

In this medical tube assembly 10, if for example a blood vessel is punctured by mistake when puncturing a living body by the puncture member 3, blood flowing out from the wound of the blood vessel flows though the through-holes 311 into the space between the outer tube 371 and the inner tube 372, and flows through the space, which helps ensure that the internal cavity of the inner tube 372 in which to dispose the implant main body 91 can be prevented from being contaminated with the blood.

According to the fourth embodiment as above, also, the same or equivalent effects to those of the aforementioned third embodiment can be produced.

FIG. 31 is a plan view showing a medical tube assembly according to a fifth embodiment of the present disclosure. Note that in FIG. 31, for convenience of explanation, a puncture member extending in a circular arc shape is depicted in the state of being stretched rectilinearly; in addition, a needle body, a state maintaining mechanism, a distal portion of an insertion section and the like are omitted from the drawing.

Referring to this figure, the fifth embodiment of a medical tube assembly will be described below. The following description will center on differences from the aforementioned first embodiment, and descriptions of the same items as above will be omitted.

As depicted in FIG. 31, in a medical tube assembly 10 in this embodiment, a main body 31 of a sheath 30, when viewed in an axial direction thereof, can include a distal separable piece (first separable tube) 32 disposed on a distal side and a proximal separable piece (second separable tube) 33 disposed on a proximal side. In accordance with an exemplary embodiment, when viewed in a radial direction thereof, the main body 31 can include an outer tube 371 and an inner tube 372 disposed on a distally inner side of the outer tube 371. A space between the outer tube 371 and the inner tube 372 can function as a flow path through which a liquid flows in a state where a puncture member 3 (medical tube assembly 10) is puncturing a living body. A side surface of the outer tube 371 is formed with a plurality of through-holes 311 communicating with the space between the outer tube 371 and the inner tube 372.

Note that an internal cavity of the inner tube 372 is an internal cavity of the main body 31, and an implant main body 91 is disposed in the internal cavity when placing the implant main body 91 indwelling in a living body.

In this medical tube assembly 10, if for example a blood vessel is punctured by mistake when puncturing a living body by the puncture member 3, blood flowing out from the wound of the blood vessel flows though the through-holes 311 into the space between the outer tube 371 and the inner tube 372, and flows through the space, which helps ensure that the internal cavity of the inner tube 372 in which to dispose the implant main body 91 can be prevented from being contaminated with blood.

According to the fifth embodiment as above, also, the same or equivalent effects to those of the aforementioned first embodiment can be produced.

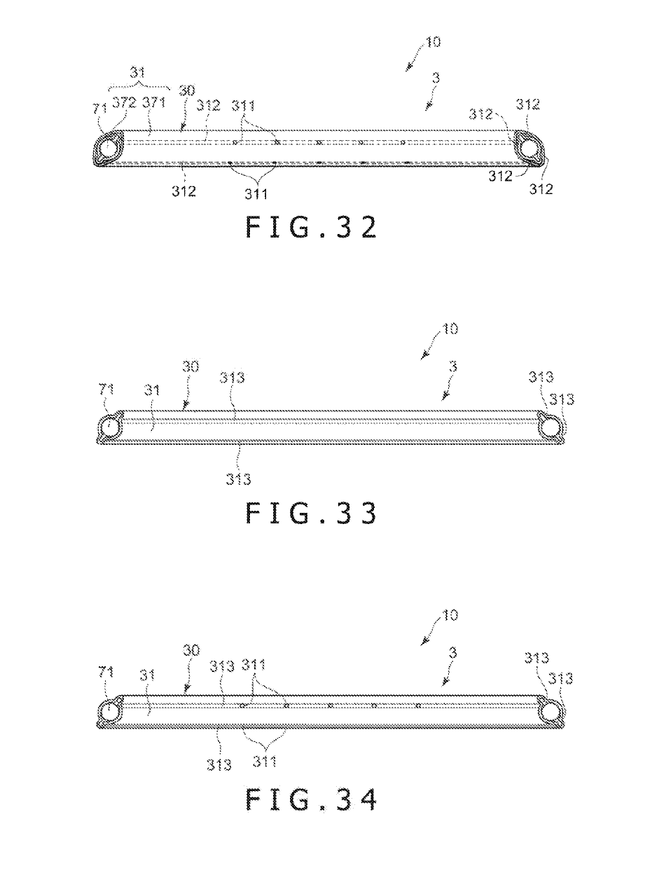

FIG. 32 is a plan view showing a medical tube assembly according to a sixth embodiment of the present disclosure. Note that in FIG. 32, for convenience of explanation, a puncture member extending in a circular arc shape is depicted in the state of being stretched rectilinearly; beside, a needle body, a state maintaining mechanism, a distal portion of an insertion section and the like are omitted from the drawing.

Referring to this figure, the sixth embodiment of a medical tube assembly will be described below. The following description will center on differences from the aforementioned fourth embodiment, and descriptions of the same items as above will be omitted.

As illustrated in FIG. 32, in a medical tube assembly 10 in this embodiment, an outer circumferential surface of an inner tube 372 constituting a main body 31 of a sheath 30 is formed with four grooves 312 extending in an axial direction of the main body 31. Each of the grooves 312, rectilinear in shape, can be formed from a distal end to a proximal end of the main body 31, and can be opening to the distal end and the proximal end. In addition, the grooves 312 are arranged along the circumferential direction of the inner tube 372. Note that the shape and layout of the grooves 312 are not limited to the ones illustrated, and other shapes may be adopted, for example, a spiral shape. In accordance with an exemplary embodiment, the number of the grooves 312 is not limited to four; for example, one, two, three, or five or more grooves may also be provided.

In addition, the side surface of the outer tube 371 is formed with a plurality of through-holes 311 communicating with each of the grooves 312. In this embodiment, a plurality of through-holes 311 are formed for each of the grooves 312. Each of the through-holes 311 is formed in a central part of the main body 31.

In this medical tube assembly 10, the grooves 312 and the through-holes 311 function as flow paths through which a liquid flows in a state where a puncture member 3 (medical tube assembly 10) is puncturing a living body. Since the flow paths are constituted of the grooves 312, the flow paths are secured assuredly, so that a liquid such as blood can flow smoothly and reliably.

According to the sixth embodiment as above, also, the same or equivalent effects to those of the aforementioned fourth embodiment can be produced.

FIG. 33 is a plan view showing a medical tube assembly according to a seventh embodiment of the present disclosure. Note that in FIG. 33, for convenience of explanation, a puncture member extending in a circular arc shape is depicted in the state of being stretched rectilinearly; in addition, a needle body, a state maintaining mechanism, a distal portion of an insertion section are omitted from the drawing.

Referring to this figure, the seventh embodiment of a medical tube assembly will be described below. The following description will center on differences from the aforementioned third embodiment, and descriptions of the same items as above will be omitted.

As shown in FIG. 33, in a medical tube assembly 10 in this embodiment, an outer circumferential surface of a main body 31 of a sheath 30 is formed with four grooves (recesses) 313 extending in an axial direction of the main body 31. Each of the grooves 313, rectilinear in shape, is formed from a distal end to a proximal end of the main body 31, and is opening to the distal end and the proximal end. The grooves 313 are arranged along the circumferential direction of the main body 31. These grooves 313 function as flow paths through which a liquid flows in a state where a puncture member 3 (medical tube assembly 10) is puncturing a living body. Note that the shape and layout of the grooves 313 are not limited to the ones illustrated, and other shapes may be adopted, for example, a spiral shape. The number of the grooves 313 is not limited to four; for example, one, two, three, or five or more grooves may also be provided.

According to the seventh embodiment as above, also, the same or equivalent effects to those of the aforementioned third embodiment can be produced.

FIG. 34 is a plan view showing a medical tube assembly according to an eighth embodiment of the present disclosure. Note that in FIG. 34, for convenience of explanation, a puncture member extending in a circular arc shape is depicted in the state of being stretched rectilinearly; besides, a needle body, a state maintaining mechanism, a distal portion of an insertion section and the like are omitted from the drawing.

Referring to this figure, the eighth embodiment of a medical tube assembly will be described below. The following description will center on differences from the aforementioned third embodiment, and descriptions of the same items as above will be omitted.

As illustrated in FIG. 34, in a medical tube assembly 10 in this embodiment, an outer circumferential surface of a main body 31 of a sheath 30 is formed with four grooves (recesses) 313 extending in an axial direction of the main body 31. Each of the grooves 313, rectilinear in shape, is formed from a distal end to a proximal end of the main body 31, and is opening to the distal end and the proximal end. The grooves 313 are arranged along the circumferential direction of the main body 31. Note that the grooves 313 may be formed, for example, only in a central part of the main body 31, instead of being formed over the whole length of the main body 31. The shape and layout of the grooves 313 are not limited to the ones illustrated, and other shapes may also be adopted, for example, a spiral shape. The number of the grooves 313 is not limited to four; for example, one, two, three, or five or more grooves 313 may also be provided.

In addition, a plurality of through-holes 311 are formed in a bottom portion of each of the grooves 313. Specifically, for example, an end portion of each of the through-holes 311 is disposed in the groove 313. By the grooves 313, the through-holes 311 can be prevented from being covered with a biological tissue when a living body is punctured by the puncture member 3. As a result, it can be relatively ensured that when a blood vessel is puncture by mistake and bleeding occurs, the blood can be reliably guided to the through-holes 311.

Note that while in this embodiment the internal cavity of the main body 31 functions as a flow path through which a liquid flows in a state where the puncture member 3 (medical tube assembly 10) is puncturing a living body, each of the grooves 313 also has the function as a flow path.

According to the eighth embodiment as above, also, the same or equivalent effects to those of the aforementioned third embodiment can be produced.

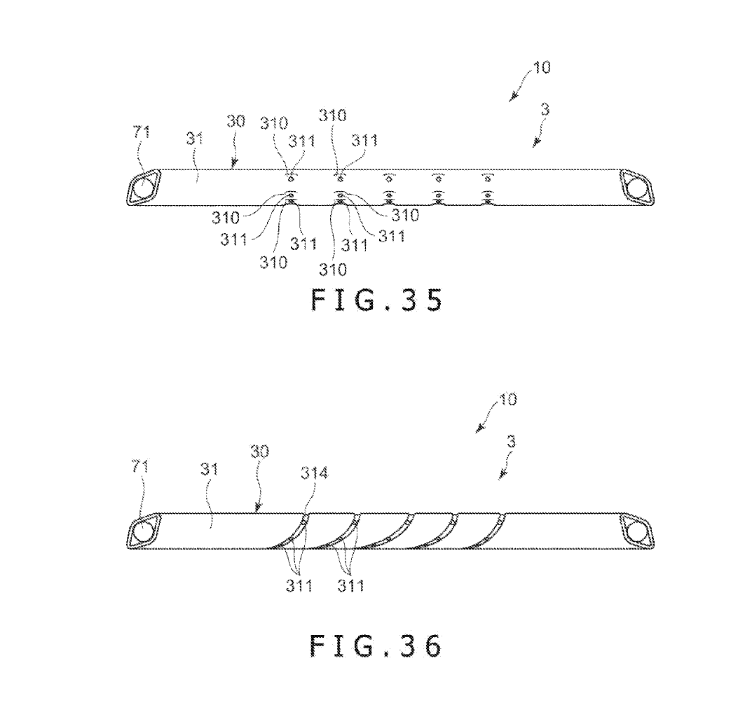

FIG. 35 is a plan view showing a medical tube assembly according to a ninth embodiment of the present disclosure. Note that in FIG. 35, for convenience of explanation, a puncture member extending in a circular arc shape is depicted in the state of being stretched rectilinearly; in addition, a needle body, a state maintaining mechanism, a distal portion of an insertion section and the like are omitted from the drawing.

Referring to this figure, the ninth embodiment of a medical tube assembly will be described below. The following description will center on differences from the aforementioned eighth embodiment, and descriptions of the same items as above will be omitted.

As shown in FIG. 35, in a medical tube assembly 10 in this embodiment, an outer circumferential surface of a main body 31 of a sheath 30 is formed with a plurality of recesses 310, in place of the rectilinear grooves 313 in the eighth embodiment. The recesses 310 are formed in a central part of the main body 31. A through-hole 311 is formed in a bottom portion in each of the recesses 310. Note that a plurality of through-holes 311 may be formed in each recess 310. In accordance with an exemplary embodiment, the recesses 310 may be formed over the whole region, or whole length, of the main body 31.

The layout of the recesses 310 is not specifically restricted, but may be appropriately set according to various conditions. In this embodiment, the recesses 310 are laid out regularly; specifically, for example, the recesses 310 are arranged at regular intervals in the axial direction of the main body 31 and at regular intervals in the circumferential direction of the main body 31. Note that other layouts of the recesses 310 include, for example, spiral layouts and irregular layouts.

According to the ninth embodiment as above, also, the same or equivalent effects to those of the aforementioned eighth embodiment can be produced.

FIG. 36 is a plan view showing a medical tube assembly according to a tenth embodiment of the present disclosure. Note that in FIG. 36, for convenience of explanation, a puncture member extending in a circular arc shape is depicted in the state of being stretched rectilinearly; besides, a needle body, a state maintaining mechanism, a distal portion of an insertion section and the like are omitted from the drawing.

Referring to this figure, the tenth embodiment of a medical tube assembly will be described below. The following description will center on differences from the aforementioned eighth embodiment, and descriptions of the same items as above will be omitted.

As illustrated in FIG. 36, in a medical tube assembly 10 in this embodiment, an outer circumferential surface of a main body 31 of a sheath 30 is formed with a spiral groove 314, in place of the four rectilinear grooves 313 in the eighth embodiment. The groove 314 is formed in a central part of the main body 31.

Note that the groove 314 may be formed over the whole region, or whole length, of the main body 31. In accordance with an exemplary embodiment, in this case, through-holes 311 may be formed over the whole length of the groove 314.

According to the tenth embodiment as above, also, the same or equivalent effects to those of the aforementioned eighth embodiment can be produced.

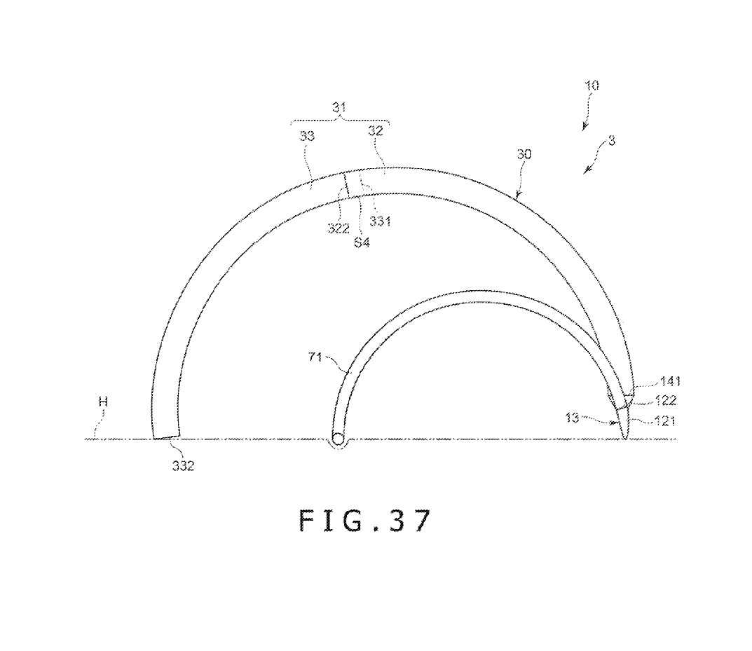

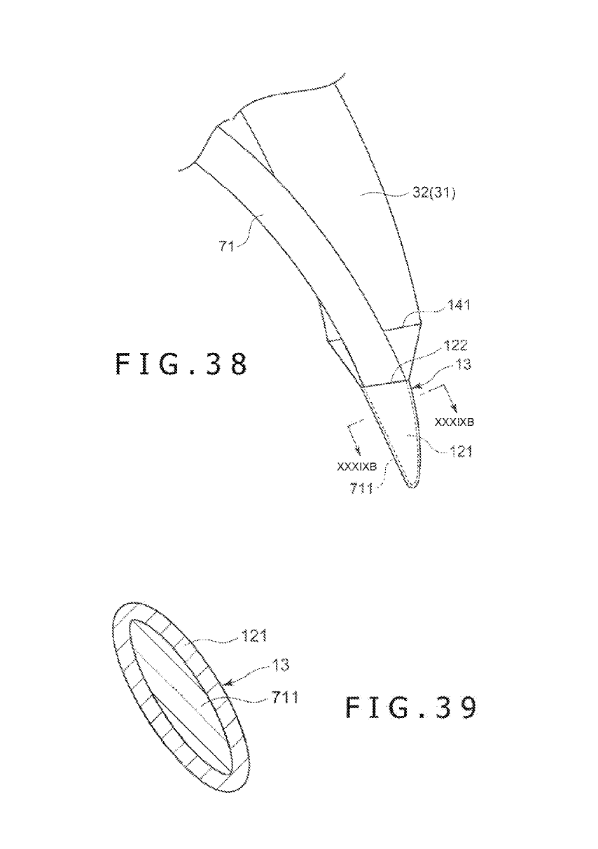

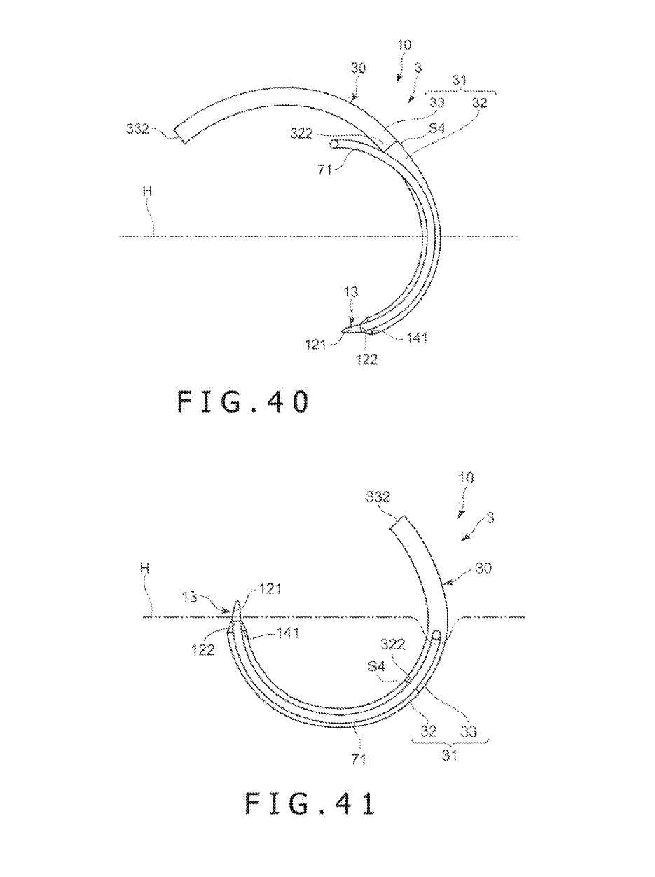

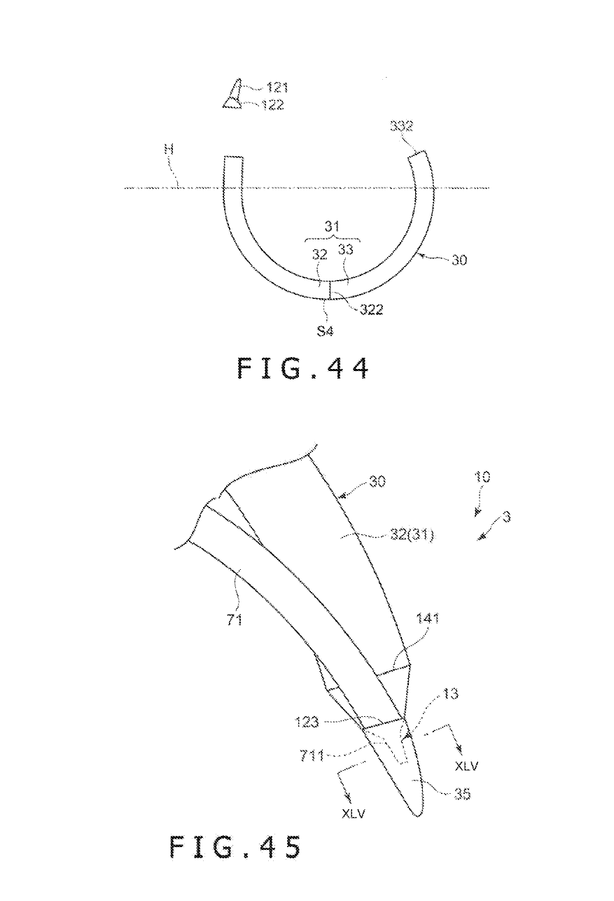

FIG. 37 is a side view showing a medical tube assembly according to an eleventh embodiment of the present disclosure. FIG. 38 is a side view showing a distal portion of the medical tube assembly shown in FIG. 37. FIG. 39 is a sectional view taken along line XXXIX-XXXIX of FIG. 38. FIGS. 40 to 44 are views for explaining a use example of the medical tube assembly shown in FIG. 37. Note that in FIGS. 37 to 44, for convenience of explanation, a state maintaining mechanism and the like are omitted from the drawing.

Referring to these figures, the eleventh embodiment of a medical tube assembly will be described below. The following description will center on differences from the aforementioned second embodiment, and descriptions of the same items as above will be omitted.

This eleventh embodiment is the same as the second embodiment above, except for differences in the configuration of sheath 30.

As illustrated in FIGS. 37 to 39, a medical tube assembly 10 in this embodiment can include a flexible bag body (recess) 121 that is an interlock portion for detachably interlocking a distal portion of a main body 31 of a sheath 30 and a distal portion 711 of an insertion section 71, and a rotation restriction unit (first rotation restriction unit) 13 that restricts rotation of the distal portion of the main body 31 and the distal portion 711 of the insertion section 71 about an axis in a state where the distal portion of the main body 31 and the distal portion 711 of the insertion section 71 are interlocked to each other.

The bag body 121 is provided at the distal portion of the main body 31. The bag body 121 is a bottomed one that has an opening 122 at one end thereof, and is closed at the other end thereof. Note that the bag body 121 is a bottomless one. Where the bag body 121 is bottomless, the distal portion 711 of the insertion section 71 can be disposed in the state of being exposed to the exterior, and a living body can be punctured directly by the distal portion 711 of the insertion section 71, so that puncture resistance is relatively low, and it can be relatively easy to puncture.

In addition, the shape of the bag body 121 corresponds to the shape of the distal portion 711 of the insertion section 71. In the configuration illustrated, a cross-sectional shape of the insertion section 71 is a flat shape, and a cross-sectional shape of the bag body 121 can also be a flat shape, correspondingly.

The distal portion 711 of the insertion section 71 is inserted into the bag body 121 via the opening 122. By this, a distal portion of the main body 31 and the distal portion 711 of the insertion section 71 are interlocked. As a result, when a puncture member 3 punctures a living body, the main body 31 is moved together with the insertion section 71. Note that the insertion section 71 is not inserted into an internal cavity of the main body 31. Hereafter, a state in which the distal portion of the main body 31 and the distal portion 711 of the insertion section 71 are interlocked to each other will be referred to also as “interlocked state.”

In accordance with an exemplary embodiment, since a cross-sectional shapes of the distal portion 711 and the bag body 121 are flat shapes, in the interlocked state the distal portion 711 and the bag body 121 can be engaged with each other in the direction of rotation of the distal portion of the main body 31 and the distal portion 711 about the axis, whereby rotation of the distal portion of the main body 31 and the distal portion 711 of the insertion section 71 about the axis is restricted. This helps ensure that when the puncture member 3 punctures a living body, the main body 31 is moved in line with the insertion section 71. As a result, the main body 31 can be reliably disposed at a position similar to a trajectory of movement of the distal portion 711 of the insertion section 71.

Note that the bag body 121 constitutes a first engaging section, and the distal portion 711 of the insertion section 71 constitutes a second engaging section for engagement with the first engaging section. In addition, the bag body 121 and the distal portion 711 of the insertion section 71 constitute a rotation restriction unit 13. Thus, in this embodiment, the rotation restriction unit 13 is provided at the distal portions of the main body 31 and the insertion section 71.

Note that in the case where a cross-sectional shape of the main body 31 is a flat shape, the rotation restriction unit 13 restricts rotation of the main body 31 and the insertion section 71 about the axis in such a manner that a major axis J32 in cross-sectional plane of the main body 31 and an axis of a urethral-insertion member 4 will be parallel to each other.

The material constituting the bag body 121 is not particularly restricted, and examples of the material include resin materials, paper, and metallic materials.

In addition, the axial length of the main body 31 is greater than the axial length of the insertion section 71. This can be realized since the insertion section 71 is not inserted in the internal cavity of the main body 31. This makes it possible to cope with, for example, a relatively large patient. Specifically, for example, by appropriately setting the axial length of the main body 31, an implant main body 91 can be placed indwelling in a living body easily and reliably, in the cases of patients of various body types. Note that, naturally, the axial length of the main body 31 may be equal to the axial length of the insertion section 71 or may be smaller than the axial length of the insertion section 71.

It is preferable for the main body 31 to be so rigid that the curvature and internal cavity of the main body 31 can be maintained in a state where the main body 31 is inserted in a living body (in a state of puncturing and passing through a living body).

It is preferable that at least part of the main body 31 is flexible. In the configuration illustrated, a part near a central portion S4 of the main body 31 is flexible, which helps enable the main body 31 to easily follow up to the insertion section 71 when the puncture member 3 punctures a living body.

In addition, a marker 141 is provided at a distal portion of the main body 31. The marker 141 is disposed in such a manner that the distance from the central portion S4 of the main body 31 to the marker 141 and the distance from the central portion S4 to the proximal end of the main body 31 are equal. The marker 141 helps enable the central portion S4 of the main body 31 to be reliably positioned between a urethra 1300 and a vagina 1400, as will be described later.

Main steps of an operating procedure of a puncture apparatus 1 will now be described below. The following description will focus on differences from the first and second embodiments.

First, an operating member 7 is operated (see FIGS. 20A and 20B), to cause the medical tube assembly 10 in the state shown in FIG. 37 to puncture a patient (see FIGS. 40 and 41). In this case, in a condition where the distal portion of the main body 31 and the distal portion 711 of the insertion section 71 are interlocked to each other, the insertion section 71 and the main body 31 are inserted into the living body while being in an aligned state.

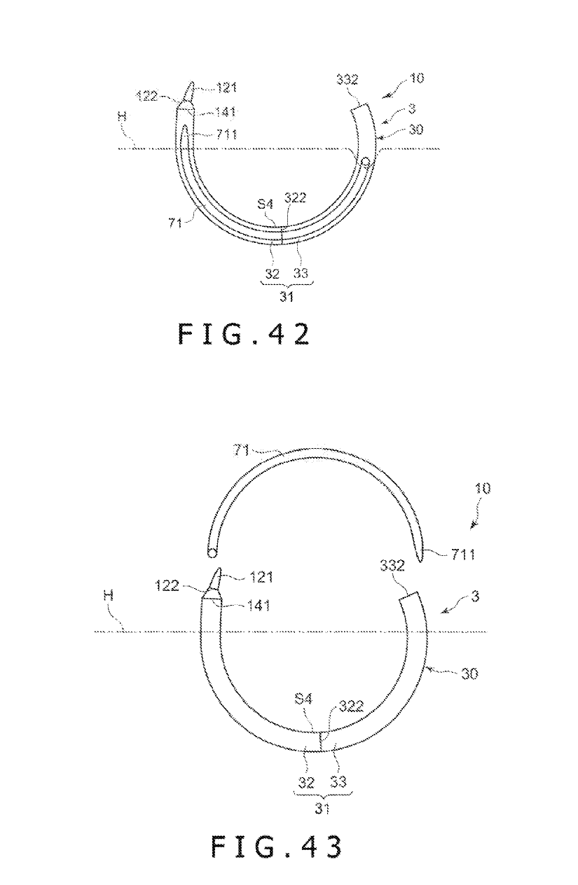

Next, as depicted in FIG. 42, the distal portion of the main body 31 or the bag body 121 is grasped and pulled, to move the main body 31 further in the distal direction. This causes the insertion section 71 to be detached from the bag body 121.

Subsequently, as shown in FIG. 43, the operating member 7 is operated to draw the insertion section 71 out of the living body.

Next, the position of the main body 31 is regulated in such a manner that the height of the marker 141 and the height of the proximal end of the main body 31 will be equal. By this operation, the central portion S4 of the main body 31 is disposed between the urethra 1300 and the vagina 1400.

Subsequently, as shown in FIG. 43, the distal portion of the main body 31 is cut, for example, at the part of the marker 141. By these operations, the main body 31 is placed in the living body. Note that the main body 31 may be so configured that the part on the distal side of the marker 141 can be detached from the part on the proximal side of the marker 141 in such a manner that the main body 31 is divided at the position of the marker 141 into the part on the distal side and the part on the proximal side.

As has been described above, according to the puncture apparatus 1, the length of the main body 31 of the sheath 30 can be made greater than the length of the insertion section 71. By appropriately setting the length of the main body 31, therefore, the implant main body 91 can be placed indwelling in a living body easily and assuredly, in the cases of patients of various body types, such as relatively large patients, for example.

In addition, in placing the implant main body 91 indwelling in a living body, the necessary operation can be coped with low-invasive operations such as puncture with the puncture member 3, without need to perform a highly invasive incision. Therefore, the burden on the patient is relatively light, and the safety of the patient is relatively high.

In accordance with an exemplary embodiment, the living body can be punctured by the puncture member 3 while avoiding the urethra 1300 and the vagina 1400. Therefore, puncturing of the urethra 1300 or the vagina 1400 by the puncture member 3 can be prevented from occurring, and, accordingly, safety can be relatively ensured.

Further, unlike in the case of conventional incision of the vagina 1400, it is possible to eliminate the possibility of occurrence of a situation in which the implant main body 91 would be exposed to the inside of the vagina 1400 via a wound caused by the incision, or a situation in which complications would be generated such as infection from the wound. Thus, very high safety can be ensured, and the implant main body 91 can be reliably embedded.

Note that while the insertion section 71 pushes the sheath 30 to move the sheath 30 in this embodiment, this is not restrictive. For example, the insertion section 71 may pull the sheath 30 to move the sheath 30.

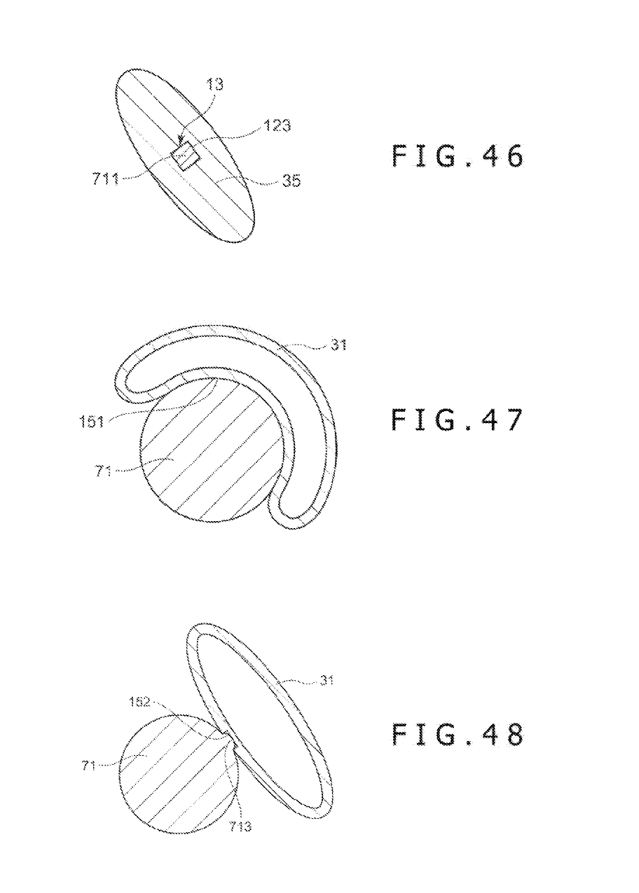

FIG. 45 is a side view showing a distal portion of a medical tube assembly according to a twelfth embodiment of the present disclosure. FIG. 46 is a sectional view taken along line XLV-XLV of FIG. 45. Note that in FIG. 45, for convenience of explanation, a state maintaining mechanism and the like are omitted from the drawing.

Referring to these figures, the twelfth embodiment of a medical tube assembly will be described below. The following description will center on differences from the aforementioned eleventh embodiment, and descriptions of the same items as above will be omitted.

As illustrated in FIGS. 45 and 46, in a medical tube assembly 10 in this embodiment, a puncture member 3 can include a sheath 30 and a needle body 35 provided at a distal end of the sheath 30, like in the first embodiment. Note that this configuration has been described in the first embodiment, and, therefore, description thereof is omitted here.

The needle body 35 is formed with a recess 123 as an interlock portion. The shape of the recess 123 corresponds to the shape of a distal portion 711 of an insertion section 71. In the configuration illustrated, a cross-sectional shape of the distal portion 711 of the insertion section 71 is a tetragon, and a cross-sectional shape of the recess 123 can also be a tetragon, correspondingly.

The distal portion 711 of the insertion section 71 is inserted in the recess 123. By this, a distal portion of a main body 31 and the distal portion 711 of the insertion section 71 are indirectly interlocked to each other through the needle body 35. In accordance with an exemplary embodiment, in the interlocked state, the distal portion 711 and the recess 123 can be engaged with each other in the direction of rotation of the distal portion of the main body 31 and the distal portion 711 of the insertion section 71 about an axis, whereby rotation of the distal portion of the main body 31 and the distal portion 711 of the insertion section 71 about the axis can be restrained.

Note that the recess 123 constitutes a first engaging section, whereas the distal portion 711 of the insertion section 71 constitutes a second engaging section for engagement with the first engaging section. In addition, the recess 123 and the distal portion 711 of the insertion section 71 constitute a rotation restriction unit 13.

According to the twelfth embodiment as above, also, the same or equivalent effects to those of the aforementioned eleventh embodiment can be produced.

FIG. 47 is a sectional view showing an intermediate part of a medical tube assembly according to a thirteenth embodiment of the present disclosure.

Referring to this figure, the thirteenth embodiment of a medical tube assembly will be described below. The following description will center on differences from the aforementioned eleventh and twelfth embodiments, and descriptions of the same items as above will be omitted.

As depicted in FIG. 47, in a medical tube assembly 10 in this embodiment, at an intermediate part in an axial direction of a main body 31 of a sheath 30, a groove (recess) 151 is formed by bending an outer circumferential surface of the main body 31 toward an inner side (central side). In addition, a cross-sectional shape of a part on a more proximal side than a distal portion 711 of an insertion section 71 is a circle so that the insertion section 71 can be engaged with the groove 151. This helps ensure that when a puncture member 3 punctures a living body, the insertion section 71 and the groove 151 engage each other, whereby rotation of the insertion section 71 and the main body 31 about an axis is restricted. Note that the groove 151 and that part of the insertion section 71 which engages the groove 151 constitute a second rotation restriction unit that restricts rotation of the insertion section 71 and the main body 31 about an axis. Thus, in this embodiment, the second rotation restriction unit is provided at side surfaces of the main body 31 and the insertion section 71. Note that the second rotation restriction unit may be provided over the whole length of a part on the more proximal side than a distal portion of the medical tube assembly 10 or may be provided over part of the whole length.

According to the thirteenth embodiment as above, also, the same or equivalent effects to those of the aforementioned eleventh and twelfth embodiments can be produced.

FIG. 48 is a sectional view showing an intermediate part of a medical tube assembly according to a fourteenth embodiment of the present disclosure.

Referring to this figure, the fourteenth embodiment of a medical tube assembly will be described below. The following description will center on differences from the aforementioned eleventh and twelfth embodiments, and descriptions of the same items as above will be omitted.

As illustrated in FIG. 48, in a medical tube assembly 10 in this embodiment, an outer circumferential surface of an intermediate part in an axial direction of a main body 31 of a sheath 30 is formed with a groove (recess) 152. In addition, a cross-sectional shape of a part on the more proximal side than a distal portion 711 of an insertion section 71 is a circle. In accordance with an exemplary embodiment, an outer circumferential surface of an intermediate part in an axial direction of the insertion section 71 is formed with a rib (projection) 713 capable of engagement with the groove 152. This helps ensure that when a puncture member 3 punctures a living body, the rib 713 of the insertion section 71 and the groove 152 engage each other, whereby rotation of the insertion section 71 and the main body 31 about an axis is restricted. Note that the groove 152 and the rib 713 constitute a second rotation restriction unit that restricts rotation of the insertion section 71 and the main body 31 about the axis. Thus, in this embodiment, the second rotation restriction unit is provided at side surfaces of the main body 31 and the insertion section 71. Note that the second rotation restriction unit may be provided over the whole length of a part on the more proximal side than a distal portion of the medical tube assembly 10 or may be provided over part of the whole length.

According to the fourteenth embodiment as above, also, the same or equivalent effects to those of the aforementioned eleventh and twelfth embodiments can be produced.

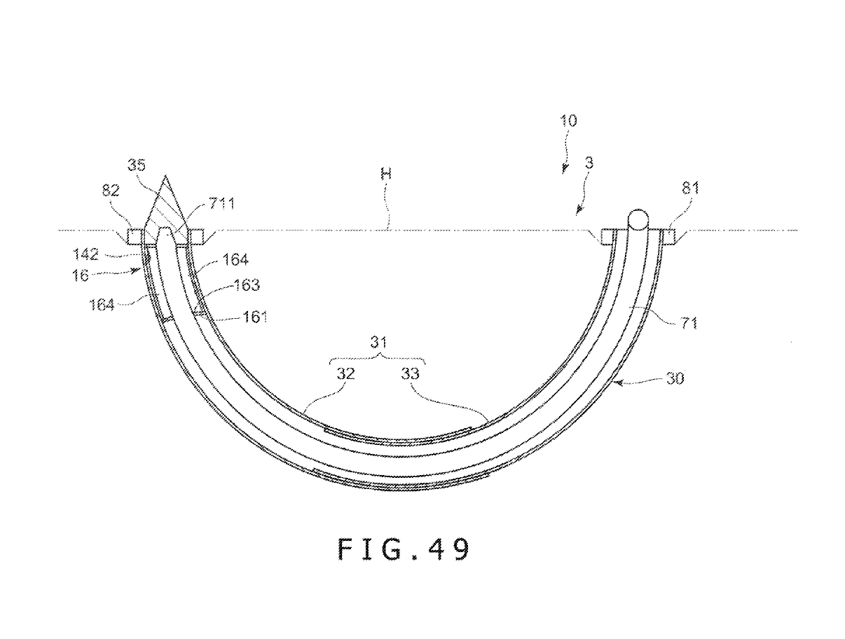

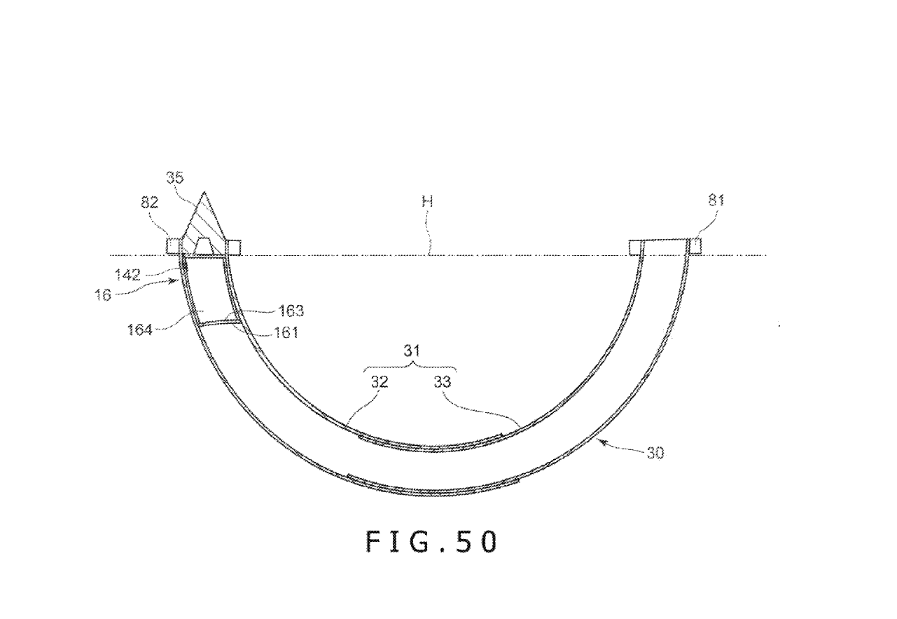

FIG. 49 is a sectional view showing a medical tube (medical tube assembly) according to a fifteenth embodiment of the present disclosure. FIGS. 50 to 52 are views for explaining a use example of the medical tube (medical tube assembly) shown in FIG. 49. Note that in FIGS. 49 to 52, for convenience of explanation, a state maintaining mechanism and the like are omitted from the drawing.

Referring to these figures, the fifteenth embodiment of a medical tube assembly will be described below. The following description will center on differences from the aforementioned first embodiment, and descriptions of the same items as above will be omitted.

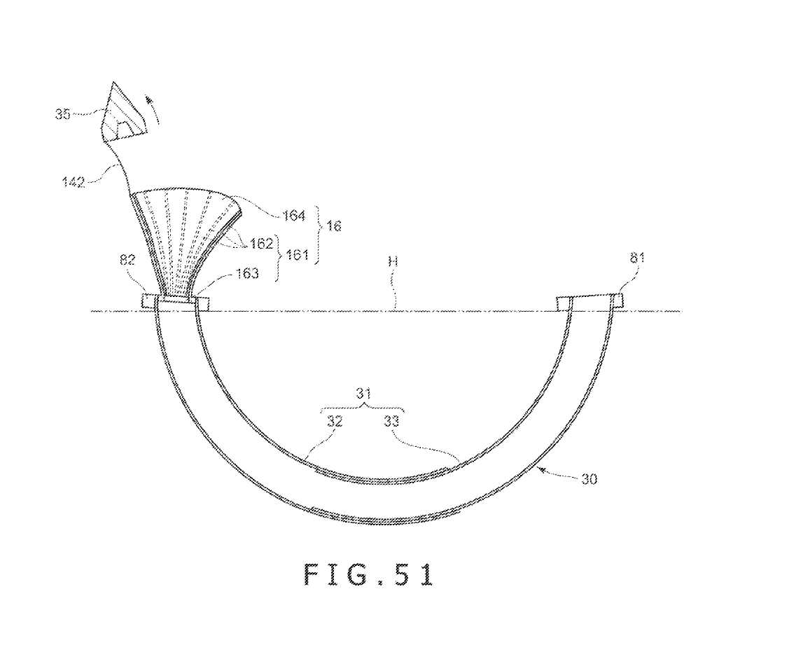

As illustrated in FIGS. 49 and 51, a sheath 30 in this embodiment can include a main body 31, a needle body 35, the state maintaining mechanism 34, and a diametrically expandable section 16 provided at a distal portion of the main body 31 and expandable in diameter as compared to a central portion of the main body 31. The diametrically expandable section 16 can be expanded and contracted in diameter. Note that the diametrically expandable section 16 may be provided at a proximal portion of the main body 31, and may be provided at both a distal portion and a proximal portion of the main body 31. In addition, the diametrically expandable section 16 may be a separate body from the main body 31. In accordance with an exemplary embodiment, the diametrically expandable section 16 may be formed to be integral with the main body 31. The diametrically expandable section 16 will be described below.

The diametrically expandable section 16 can include a skeleton member 161, and a flexible sheet 164 supported by the skeleton member 161. The skeleton member 161 can include a plurality of rib parts 162 rectilinear in shape and elastically deformable, and an annular support section 163 that supports proximal portions of the rib parts 162. The support section 163 is provided at a distal portion of the main body 31 in such a manner as to be movable in an axial direction of the main body 31, and the rib parts 162 are arranged at regular intervals along a circumferential direction of the support section 163. Note that the rib parts 162 and the support section 163 may be integral with each other or may be separate bodies from each other. The sheet 164 is fixed to the rib parts 162 on an inner portion of the rib parts 162.

First, based on FIG. 51, a diametrically expanded state of the diametrically expandable section 16 will be described. In a natural state where no external force is exerted, each of the rib parts 162 has its distal portion located on the outer side than its proximal portion as viewed in an axial direction of the main body 31 (support section 163); when the rib parts 162 are viewed as a whole, their distal portions are spread to the more outer side than their proximal portions. In addition, each rib part 162 is located on the more distal side than the distal portion of the main body 31, specifically, located outside of the main body 31. Note that a proximal portion of each rib part 162 may be located inside the main body 31. In this state, the sheet 164 is fixed to the rib parts 162 in such a manner that it assumes a tubular shape with a distal portion diametrically enlarged as compared with a proximal portion. Note that an inside diameter of the diametrically expandable section 16 gradually increases along a direction for spacing away from the main body 31 along the axial direction of the main body 31.

The material constituting the rib parts 162 is not specifically restricted insofar as the material is elastically deformable. For example, superelastic alloys such as Ni—Ti alloys are preferably used as the material.

The material constituting the sheet 164 is not particularly limited so long as the material is flexible. Examples of the applicable material include flexible resin materials and papers.

As shown in FIGS. 49 and 50, the diametrically expandable section 16 as above is accommodated in an internal cavity of a distal portion of the main body 31 in a diametrically contracted state, specifically with the rib parts 162 elastically deformed, in such a manner that the diametrically expandable section 16 is movable in the axial direction of the main body 31.

Note that the rib parts 162 function as a biasing section for biasing the diametrically contracted diametrically expandable section 16 in a direction for expanding in diameter. In addition, a distal portion of the main body 31 functions as a retaining section for retaining the diametrically expandable section 16 in a diametrically contracted state against a biasing force of the biasing section.

In accordance with an exemplary embodiment, the needle body 35 is attached to a distal portion of the main body 31 in a freely detachable manner. The needle body 35 and a distal portion of the diametrically expandable section 16 are interlocked to each other by a string 142. Note that a distal portion of the string 142 is fixed to or supported by a proximal portion of the needle body 35, and a proximal portion of the string 142 is fixed to or supported by a distal portion of the diametrically expandable section 16.

At the time of detaching the needle body 35 from a distal portion of the main body 31, if the needle body 35 is detached from the distal portion of the main body 31 and thereafter the needle body 35 is pulled further in the distal direction, a distal portion of the diametrically expandable section 16 is distally pulled by the needle body 35 through the string 142, so that the diametrically expandable section 16 is protruded from within the main body 31 to the outside. As a result, the rib parts 162 return into their natural state under their own elastic forces, so that the diametrically expandable section 16 is expanded in diameter. Thus, the needle body 35 can function also as an operation section for operation to diametrically expand the diametrically expandable section 16, and the diametrically expandable section 16 is expanded in diameter in conjunction with a detaching operation of detaching the needle body 35 from the distal portion of the main body 31. Note that the total length of the main body 31 is enlarged through the process in which the diametrically expandable section 16 is protruded from the distal portion of the main body 31 and is expanded in diameter.

Now, main steps of an operating procedure of a puncture apparatus 1 will be described below. The following description will focus on differences from the first embodiment.

First, as shown in FIG. 49, the operating member 7 is operated (see FIGS. 20A and 20B) to cause the medical tube assembly 10 to puncture a patient.

Next, as illustrated in FIG. 50, the operating member 7 is operated to draw the insertion section 71 out of the living body. As a result of these operations, the main body 31 is disposed inside the living body.

Subsequently, as shown in FIG. 51, the needle body 35 is detached from the distal portion of the main body 31, and the needle body 35 is pulled distally. By this operation, the distal portion of the diametrically expandable section 16 is pulled distally by the needle body 35 through the string 142, and the diametrically expandable section 16 is protruded from within the main body 31 to the outside. As a result, the rib parts 162 return into their natural state under their own elastic forces, and the diametrically expandable section 16 is expanded in diameter.

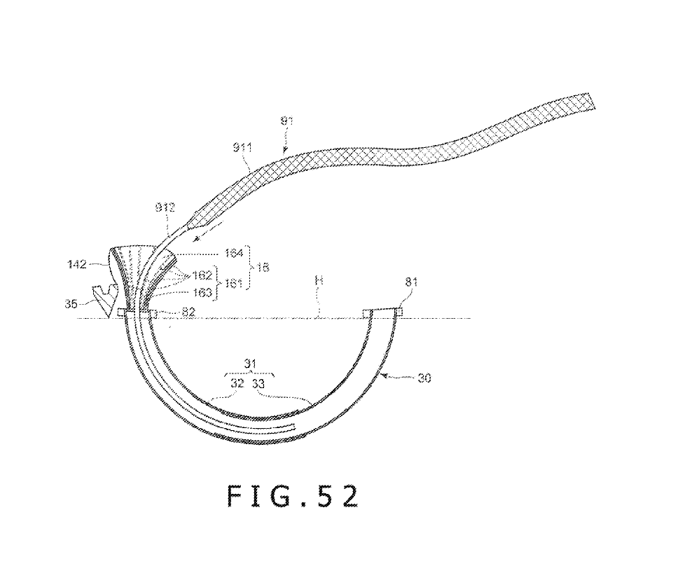

Next, as shown in FIG. 52, an implant main body 91 is inserted into the main body 31 by way of the diametrically expandable section 16. In this instance, since the diametrically expandable section 16 is in a diametrically expanded state, the implant main body 91 can be inserted into the main body 31 relatively easily and smoothly.

As has been described above, according to the puncture apparatus 1, since the diametrically expandable section 16 is provided, the implant main body 91 can be inserted into the main body 31 relatively easily and reliably even if the main body 31 is reduced in diameter. Consequently, the implant main body 91 can be placed indwelling in a living body relatively easily and assuredly.

In addition, with the main body 31 reduced in diameter, the burden on the patient can be lightened.

An operation of placing the implant main body 91 indwelling in a living body can be coped with by only low-invasive operations such as puncture with the puncture member 3, without need to perform highly invasive incision or the like. Therefore, the burden on the patient is relatively light, and the safety of the patient is relatively high.

In addition, a living body can be punctured by the puncture member 3 while avoiding a urethra 1300 and a vagina 1400. Therefore, puncturing of the urethra 1300 or the vagina 1400 by the puncture member 3 can be prevented from occurring. Thus, safety can be ensured.

Further, unlike in the case of conventional incision of the vagina, it is possible to eliminate the possibility of occurrence of a situation in which the implant main body 91 would be exposed to the inside of the vagina via a wound caused by the incision, or a situation in which complications would be generated such as infection from the wound. Thus, relatively high safety can be ensured, and the implant main body 91 can be reliably embedded.

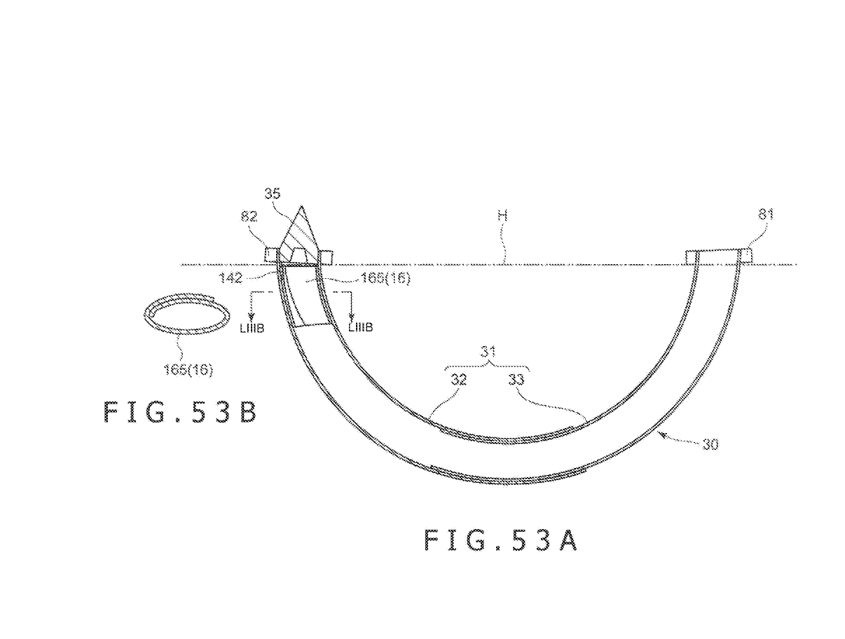

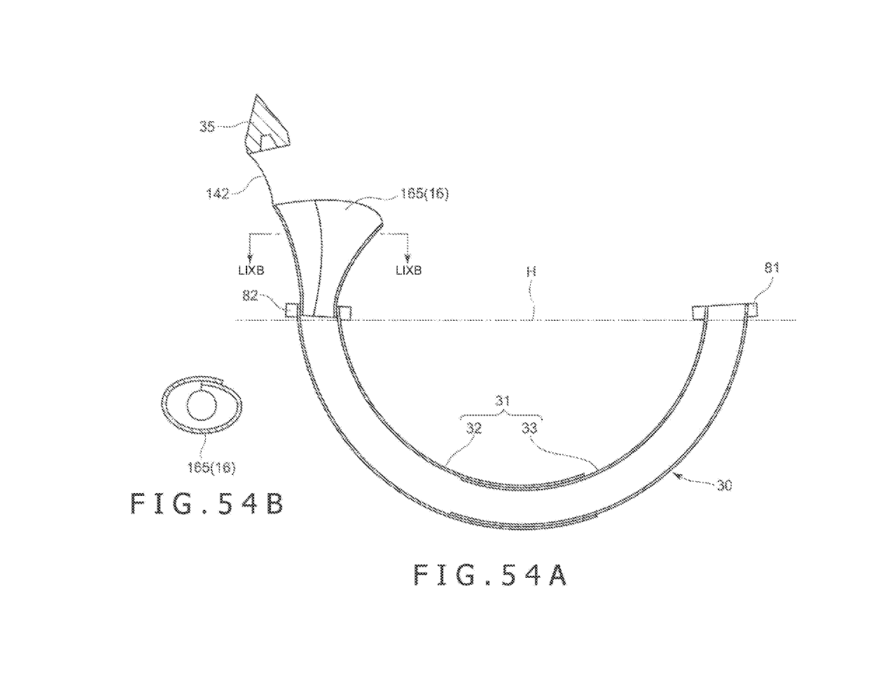

FIGS. 53A and 53B illustrate a medical tube (medical tube assembly) according to a sixteenth embodiment of the present disclosure, wherein FIG. 53A is a sectional view, and FIG. 53B is a sectional view taken along line LIIIB-LIIIB of FIG. 53A. FIGS. 54A and 54B are views for explaining a use example of the medical tube (medical tube assembly) shown in FIGS. 53A and 53B, wherein FIG. 54A is a sectional view, and FIG. 54B is a sectional view taken along line LIXB-LIXB of FIG. 54A. Note that in FIGS. 53A, 53B, 54A and 54B, for convenience of explanation, a state maintaining mechanism and the like are omitted from the drawing.

Referring to these figures, the sixteenth embodiment of a medical tube assembly will be described below. The following description will center on differences from the aforementioned fifteenth embodiment, and descriptions of the same items as above will be omitted.

As illustrated in FIGS. 53A, 53B, 54A and 54B, in a sheath 30 in this embodiment, a diametrically expandable section 16 has a flexible and elastically deformable sheet 165.

First, based on FIGS. 54A and 54B, a diametrically expanded state of the diametrically expandable section 16 will be described. In a natural state where no external force is exerted, the sheet 165 has a tubular shape with a distal portion enlarged in diameter as compared with a proximal portion. The sheet 165 can expand diametrically from a diametrically contracted state by its own restoring force (elastic force).

The material constituting the sheet 165 is not specifically restricted so long as the material is flexible and elastically deformable. Examples of the applicable material include flexible resin materials and papers.

As depicted in FIGS. 53A and 53B, the sheet 165, or the diametrically expandable section 16, is accommodated in an internal cavity of a distal portion of a main body 31 in a diametrically contracted state, or in a state of being rounded in a cylindrical form, in such a manner as to be movable along an axial direction of the main body 31.

Note that the sheet 165 functions as a biasing section for biasing the sheet 165 itself, or the diametrically expandable section 16 in a diametrically contracted state, in a direction for expanding diametrically.

At the time of detaching the needle body 35 from a distal portion of the main body 31, if the needle body 35 is detached from the distal portion of the main body 31 and thereafter the needle body 35 is further distally pulled, a distal portion of the diametrically expandable section 16 is distally pulled by the needle body 35 through a string 142, so that the diametrically expandable section 16 is protruded from within the main body 31 to the outside. As a result, the sheet 165 returns into its natural state under its own restoring force, whereby the sheet 165, or the diametrically expandable section 16, is expanded diametrically.

According to the sixteenth embodiment as above, also, the same or equivalent effects to those of the aforementioned fifteenth embodiment can be produced.

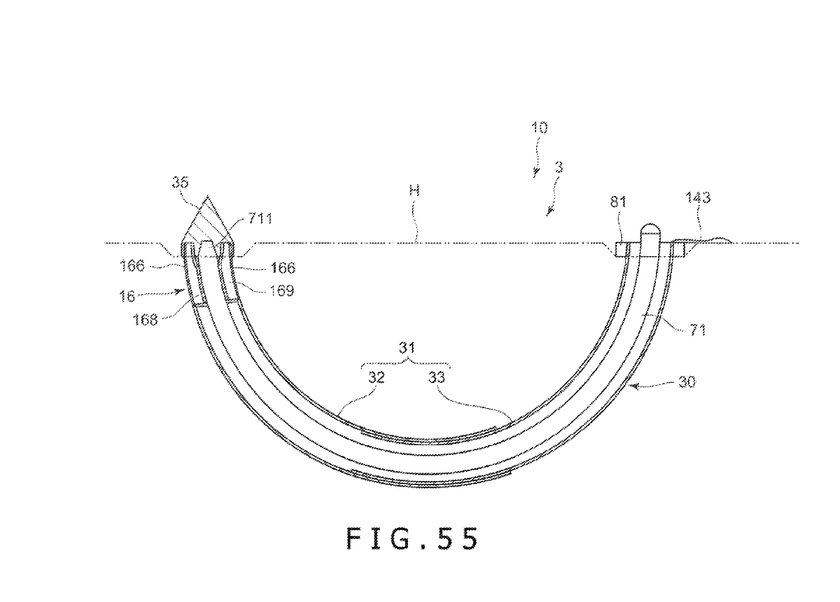

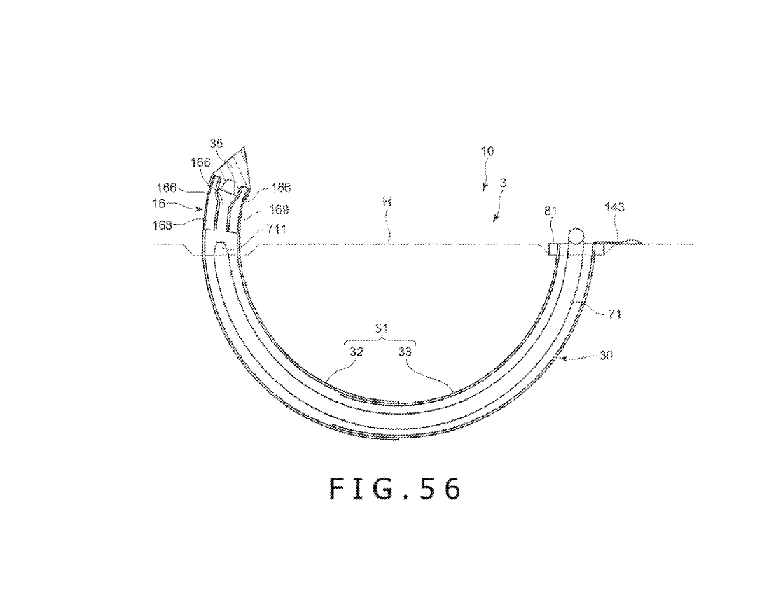

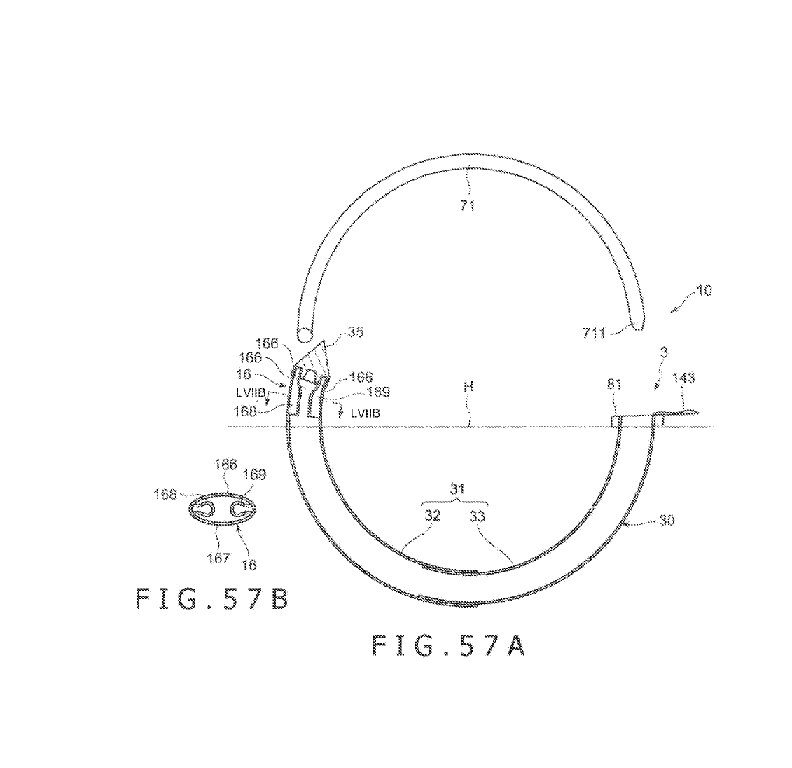

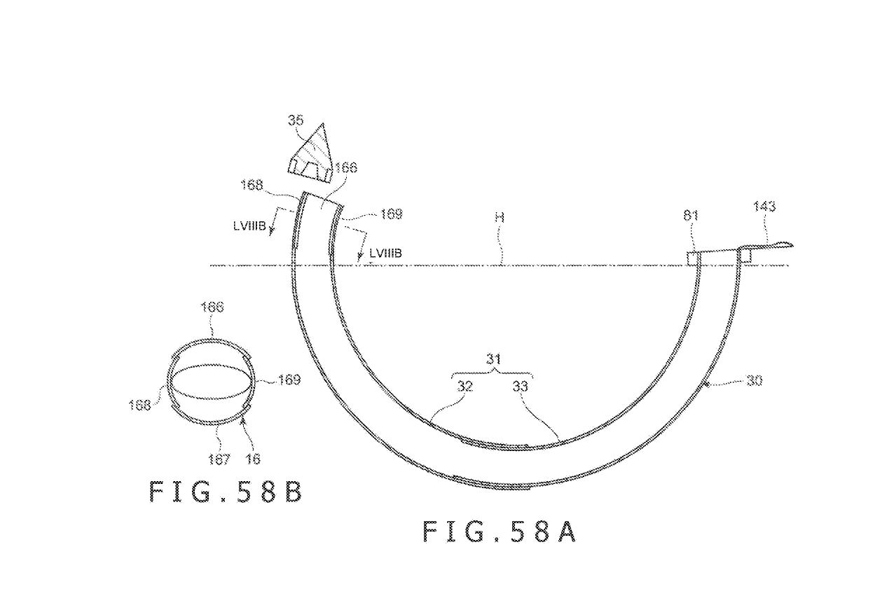

FIG. 55 is a sectional view showing a medical tube (medical tube assembly) according to a seventeenth embodiment of the present disclosure. FIG. 56 is a view for explaining a use example of the medical tube (medical tube assembly) shown in FIG. 55. FIGS. 57A and 57B are views for explaining the use example of the medical tube (medical tube assembly) shown in FIG. 55, wherein FIG. 57A is a sectional view, and FIG. 57B is a sectional view taken along line LVIIB-LVIIIB of FIG. 57A. FIGS. 58A and 58B are views for explaining the use example of the medical tube (medical tube assembly) shown in FIG. 55, wherein FIG. 58A is a sectional view, and FIG. 58B is a sectional view taken along line LVIIIB-LVIIIB of FIG. 58A. Note that in FIGS. 55 to 58B, for convenience of explanation, a state maintaining mechanism and the like are omitted from the drawing.

Referring to these figures, the seventeenth embodiment of a medical tube assembly will be described below. The following description will center on differences from the aforementioned fifteenth embodiment, and descriptions of the same items as above will be omitted.

As illustrated in FIGS. 57A, 57B, 58A and 58B, in a sheath 30 in this embodiment, a diametrically expandable section 16 can include a pair of divisional pieces 166 and 167 formed by bisecting a tubular member in a radial direction and elastically deformable, an interlock sheet 168 interlocking one-side end portions of the divisional piece 166 and the divisional piece 167 and being flexible, and an interlock sheet 169 interlocking other-side end portions of the divisional piece 166 and the divisional piece 167 and being flexible. Proximal ends of the divisional pieces 166 and 167 are fixed to a distal end of a main body 31. Note that the divisional pieces 166 and 167 and the main body 31 may be formed to be integral with each other, or may be separate bodies from each other.

First, based on FIGS. 58A and 58B, a diametrically expanded state of the diametrically expandable section 16 will be described. In a natural state where no external force is exerted, each of the divisional pieces 166 and 167 has its distal portion located on an outer side than its proximal portion as viewed in an axial direction of the main body 31. When the divisional pieces 166 and 167 are viewed as a whole, their distal portions are spread to the outer side rather than their proximal portions.

The material constituting the divisional pieces 166 and 167 is not specifically restricted so long as the material is elastically deformable. Examples of the applicable material include flexible resin materials.

The material constituting the interlock sheets 168 and 169 is not particularly limited so long as the material is flexible. For example, flexible resin materials, and papers may be used.

In a state where the diametrically expandable section 16 is contracted in diameter, specifically in a state where the divisional pieces 166 and 167 are elastically deformed, as shown in FIGS. 57A and 57B, the needle body 35 is attached to a distal portion of the diametrically expandable section 16 in a freely detachable manner. In other words, the needle body 35 is indirectly attached to a distal portion of the main body 31 through the diametrically expandable section 16. In addition, with the needle body 35 attached to the diametrically expandable section 16, the diametrically expandable section 16 is retained in a diametrically contracted state. Thus, the needle body 35 functions also as a retaining section. In accordance with an exemplary embodiment, the divisional pieces 166 and 167 function also as a biasing section for biasing the diametrically contracted diametrically expandable section 16 in a direction for expanding diametrically.

When the needle body 35 is detached from the diametrically expandable section 16, the divisional pieces 166 and 167 return into their natural state under their own elastic forces, whereby the diametrically expandable section 16 is expanded diametrically.

In addition, a string 143 is connected to a proximal portion of the proximal separable piece 33.

Now, main steps of an operating procedure of the puncture apparatus 1 will be described below. The following description will focus on differences from the first embodiment.

First, as shown in FIG. 55, the operating member 7 is operated (see FIGS. 20A and 20B), to cause the medical tube assembly 10 to puncture a patient.

Next, as illustrated in FIG. 56, only the distal separable piece 32 is further moved in the distal direction.

Subsequently, as shown in FIGS. 57A and 57B, the operating member 7 is operated to drawn the insertion section 71 out of the living body. As a result of these operations, the main body 31 is disposed inside the living body.

Next, as shown in FIGS. 58A and 58B, the needle body 35 is detached from the distal portion of the main body 31. As a result, the divisional pieces 166 and 167 return into their natural state under their own elastic forces, whereby the diametrically expandable section 16 is expanded diametrically.

Subsequently, the implant main body 91 is inserted into the main body 31 by way of the diametrically expandable section 16. In this instance, since the diametrically expandable section 16 is in a diametrically expanded state, the implant main body 91 can be inserted into the main body 31 relatively easily and smoothly.

According to the seventeenth embodiment as above, also, the same or equivalent effects to those of the aforementioned fifteenth embodiment can be produced.

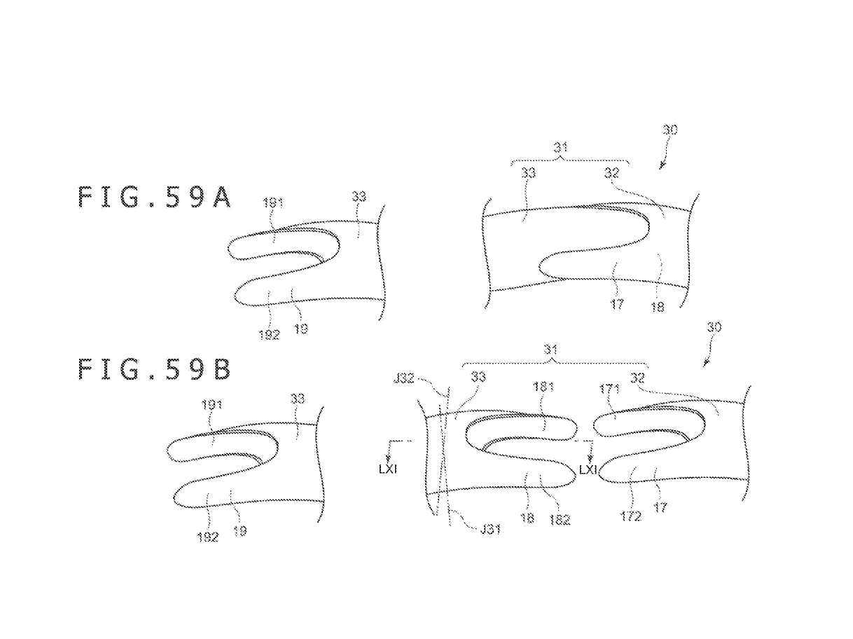

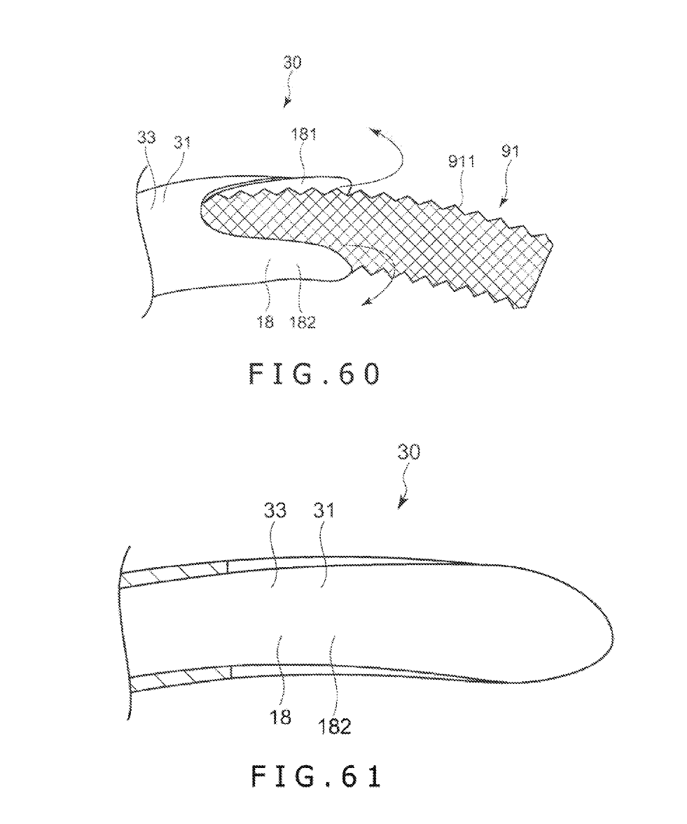

FIGS. 59A and 59B are perspective views showing a medical tube according to an eighteenth embodiment of the present disclosure, wherein FIG. 59A is a perspective view showing an assembled state of a distal separable piece and a proximal separable piece, and FIG. 59B is a perspective view showing a separated state of the distal separable piece and the proximal separable piece. FIG. 60 is a view for explaining a use example of the medical tube shown in FIGS. 59A and 59B. FIG. 61 is a sectional view taken along line LXI-LXI of FIG. 59B.

Note that in the following, for convenience of explanation, the right side in FIGS. 59A to 61 will be referred to as “distal side,” the left side as “proximal side,” the upper side as “upper side,” and the lower side as “lower side.” In FIGS. 59A to 61, for convenience of explanation, a sheath (medical tube) extending in a circular arc shape is depicted in the state of being stretched rectilinearly; further, in these figures, for convenience of explanation, a state maintaining mechanism and the like are omitted from the drawing.

Referring to these figures, the eighteenth embodiment of a medical tube assembly will be described below. The following description will center on differences from the aforementioned first embodiment, and descriptions of the same items as above will be omitted.

As illustrated in FIGS. 59A and 59B, in a sheath 30 in this embodiment, a distal separable piece 32 of a main body 31 is provided, at its end portion (proximal end portion) on a proximal separable piece 33 side, with a flexible portion (first flexible portion) 17 more flexible than the end portion. The flexible portion 17 is the part which is the most flexible of the distal separable piece 32.

Also, the proximal separable piece 33 is provided, at its end portion (distal end portion) on the distal separable piece 32 side, with a flexible portion (second flexible portion) 18 more flexible than the end portion.

Further, the proximal separable portion 33 is provided, at its end portion (proximal end portion) on the opposite side from the distal separable piece 32, with a flexible portion (third flexible portion) 19 more flexible than the end portion. The flexible portion 19 and the flexible portion 18 are the parts that are most flexible of the proximal separable piece 33 (the flexible portion 18 is more flexible than the part, on the central portion side of the flexible portion 18, of the proximal separable piece 33, and the flexible portion 19 is more flexible than the part, on the central portion side of the flexible portion 19, of the proximal separable piece 33). Note that while the degrees of flexibleness of the flexible portion 18 and the flexible portion 19 are the same in this embodiment, the degrees of flexibleness may be different.

Note that the distal separable piece 32 may be provided, at its end portion (distal end portion) on the opposite side from the proximal separable piece 33, with a flexible portion (fourth flexible portion) that is more flexible than its central portion, namely, for example, that is most flexible, in place of the flexible portion 19. Further, the distal separable piece 32 may be provided with both the flexible portion 19 and the fourth flexible portion.

In addition, the flexible portion 17 can include a pair of tongue pieces (first tongue pieces) 171 and 172 being elastic, disposed opposite to each other with a center axis of the distal separable piece 32 therebetween, and elastically deformable in directions for spacing away from each other.

Similarly, the flexible portion 18 can include a pair of tongue pieces (second tongue pieces) 181 and 182 being elastic, disposed opposite to each other with a center axis of the proximal separable piece 33 therebetween, and elastically deformable in directions for spacing away from each other.

Further, the flexible portion 19 can include a pair of tongue pieces (third tongue pieces) 191 and 192 being elastic, disposed opposite to each other with the center axis of the proximal separable piece 33 therebetween, and elastically deformable in directions for spacing away from each other.

As shown in FIG. 59B, the tongue piece 181 and the tongue piece 182 of the flexible portion 18 are aligned along the direction of the major axis J32.

In addition, the flexible portion 18 has a part where the degree of flexibleness gradually increases from the side of a central portion of the proximal separable piece 33 toward the side of an end portion (distal side) on the distal separable piece 32 side (a part where the degree of flexibleness gradually decreases toward the side of the central portion). In this embodiment, as depicted in FIG. 61, the thickness of the tongue piece 182 gradually decreases from the proximal side toward the distal side, and, similarly, the thickness of the tongue piece 181 gradually decreases from the proximal side toward the distal side.

In accordance with an exemplary embodiment, the tongue piece 171 and the tongue piece 172 of the flexible portion 17 are aligned along the direction of the major axis J32.

In addition, the flexible portion 17 has a part where the degree of flexibleness gradually increases from the side of a central portion of the distal separable piece 32 toward the side of an end portion (proximal side) on the proximal separable piece 33 side (a part where the degree of flexibleness gradually decreases toward the side of the central portion). In this embodiment, the thickness of the tongue piece 172 gradually decreases from the distal side toward the proximal side, and, similarly, the thickness of the tongue piece 171 gradually decreases from the distal side toward the proximal side.

In accordance with an exemplary embodiment, the tongue piece 191 and the tongue piece 192 of the flexible portion 19 are aligned along the direction of the major axis J32.

In addition, the flexible portion 19 has a part where the degree of flexibleness gradually increases from the side of a central portion of the proximal separable piece 33 toward the side of an end portion (proximal side on the opposite side from the distal separable piece 32 (a part where the degree of flexibleness gradually decreases toward the side of the central portion). In this embodiment, the thickness of the tongue piece 192 gradually decreases from the distal side toward the proximal side, and, similarly, the thickness of the tongue piece 191 gradually decreases from the distal side toward the proximal side.

The material or materials constituting the flexible portions 17, 18 and 19 are not specifically restricted. For example, flexible resin materials may be used.

Note that the flexible portion 17 and other portions than the flexible portion 17, of the distal separable piece 32, may be formed from the same material or may be formed from different materials. Similarly, the flexible portions 18 and 19 and other portions than the flexible portions 18 and 19, of the proximal separable piece 33, may be formed from the same material or may be formed from different materials.

These flexible portions 17 to 19 are each deformed when making contact with the implant main body 91. Specifically, for example, when the flexible portions 17 to 19 each receive a force from the implant main body 91, the flexible portions 17 to 19 each deform in a direction for relieving the force.

Specifically, for example, as depicted in FIG. 60, when the distal separable piece 32 and the proximal separable piece 33 are separated from each other and the implant main body 91 is discharged from within the main body 31, the tongue pieces 181 and 182 of the flexible portion 18 of the proximal separable piece 33 elastically deform in directions for spacing away from each other, namely, in such a manner as to open wider in the direction of the major axis J32. Similarly, the tongue pieces 171 and 172 of the flexible portion 17 of the distal separable piece 32 elastically deform in directions for spacing away from each other, namely, in such a manner as to open wider in the direction of the major axis J32, which helps enable the implant main body 91 to be discharged from within the main body 31 relatively easily, smoothly and reliably.

In addition, when the implant main body 91 is inserted into the main body 31 from a proximal end portion of the main body 31, the tongue pieces 191 and 192 of the flexible portion 19 of the proximal separable piece 33 elastically deform in directions for spacing away from each other, which helps enable the implant main body 91 to be inserted into the main body 31 via the proximal end portion of the proximal separable piece 33 relatively easily, smoothly and assuredly.

Note that it is preferable, for example, for the main body 31 to be so rigid that the curvature and an internal cavity of the main body 31 can be maintained in a state where the main body 31 is inserted in a living body (in a state where the main body 31 is puncturing and passing through a living body). In accordance with an exemplary embodiment, in a state where the distal separable piece 32 and the proximal separable piece 33 of the main body 31 are in connection with each other, the internal cavity of the main body 31 is in a communicating state over the whole length of the main body 31.

As has been described above, according to the puncture apparatus 1, the flexible portion 19 is provided at the proximal end portion of the main body 31, so that the implant main body 91 can be inserted into the main body 31 via the proximal end portion of the main body 31 relatively easily, smoothly and reliably. Consequently, the implant main body 91 can be placed indwelling in a living body relatively easily and reliably.

In addition, since the distal separable piece 32 has the flexible portion 17 at its end portion on the proximal separable piece 33 side and the proximal separable piece 33 has the flexible portion 18 on its end portion on the distal separable piece 32 side, the implant main body 91 can be discharged from within the main body 31 relatively easily, smoothly and assuredly. As a consequence, the implant main body 91 can be placed indwelling in a living body relatively easily and reliably.

An operation of placing the implant main body 91 indwelling in a living body can be coped with by only low-invasive operations such as puncture with the puncture member 3, without need to perform highly invasive incision. Therefore, the burden on the patient is relatively light, and the safety of the patient is relatively high.

In addition, a living body can be punctured by the puncture member 3 while avoiding a urethra 1300 and a vagina 1400. Therefore, puncturing of the urethra 1300 or the vagina 1400 by the puncture member 3 can be prevented from occurring. Thus, safety can be relatively ensured.

Further, unlike in the case of conventional incision of the vagina, it is possible to eliminate the possibility of occurrence of a situation in which the implant main body 91 would be exposed to the inside of the vagina via a wound caused by the incision, or a situation in which complications would be generated such as infection from the wound. Thus, very high safety can be ensured, and the implant main body 91 can be reliably embedded.

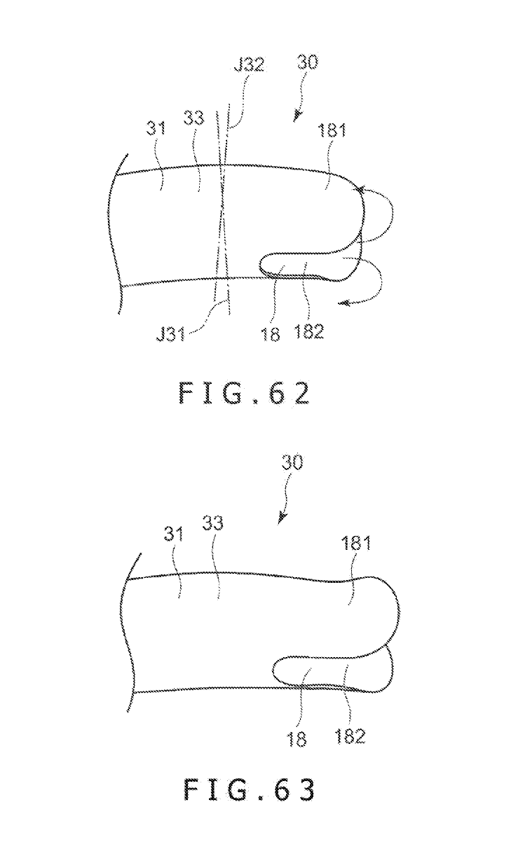

FIG. 62 is a perspective view showing a medical tube according to a nineteenth embodiment of the present disclosure. FIG. 63 is a view for explaining a use example of the medical tube shown in FIG. 62. Note that in the following, for convenience of explanation, the right side in FIGS. 62 and 63 will be referred to as “distal side,” the left side as “proximal side,” the upper side as “upper side,” and the lower side as “lower side.” In addition, in FIGS. 62 and 63, for convenience of explanation, a sheath (medical tube) extending in a circular arc shape is depicted in the state of being stretched rectilinearly. Further, in FIGS. 62 and 63, for convenience of explanation, a state maintaining mechanism and the like are omitted from the drawing.

Referring to these figures, the nineteenth embodiment of a medical tube assembly will be described below. The following description will center on differences from the aforementioned eighteenth embodiment, and descriptions of the same items as above will be omitted.

As illustrated in FIG. 62, in a sheath 30 in this embodiment, a tongue piece 181 and a tongue piece 182 of a flexible portion 18 are aligned in the direction of a minor axis J31. Similarly, a tongue piece 171 and a tongue piece 172 of a flexible portion 17 are aligned in the direction of the minor axis J31. Further, a tongue piece 191 and a tongue piece 192 of a flexible portion 19 are aligned in the direction of the minor axis J31.

When the distal separable piece 32 and the proximal separable piece 33 are separated from each other and the implant main body 91 is discharged from within the main body 31, the tongue pieces 181 and 182 of the flexible portion 18 of the proximal separable piece 33 elastically deform in directions for spacing away from each other, namely, in such a manner as to open wider in the direction of the minor axis J31. Similarly, the tongue pieces 171 and 172 of the flexible portion 17 of the distal separable piece 32 elastically deform in directions for spacing away from each other, namely, in such a manner as to open wider in the direction of the minor axis J31, which helps enable the implant main body 91 to be discharged from within the main body 31 relatively easily, smoothly and reliably.

According to the nineteenth embodiment as above, also, the same or equivalent effects to those of the aforementioned eighteenth embodiment can be produced.

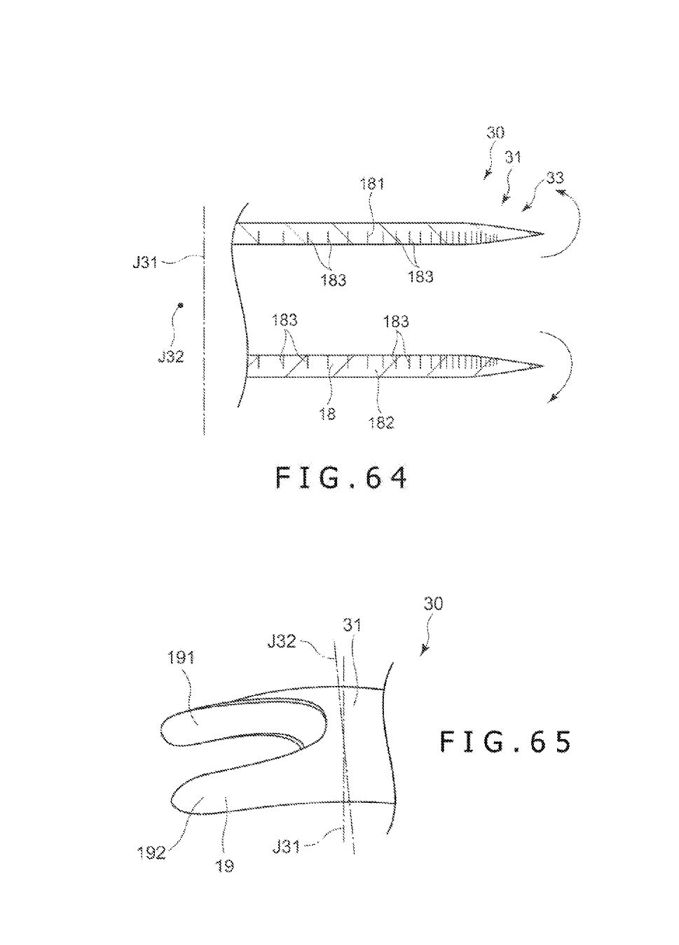

FIG. 64 is a sectional view showing a medical tube according to a twentieth embodiment of the present disclosure. Note that in the following, for convenience of explanation, the right side in FIG. 64 will be referred to as “distal side,” the left side as “proximal side,” the upper side as “upper side,” and the lower side as “lower side.” In FIG. 64, for convenience of explanation, a sheath (medical tube) extending in a circular arc shape is depicted in the state of being stretched rectilinearly. Further, in FIG. 64, for convenience of explanation, a state maintaining mechanism and the like are omitted from the drawing.

Referring to this figure, the twentieth embodiment of a medical tube assembly will be described below. The following description will center on differences from the aforementioned nineteenth embodiment, and descriptions of the same items as above will be omitted.

As illustrated in FIG. 64, in a sheath 30 in this embodiment, mutually facing surfaces of tongue pieces 181 and 182 of a flexible portion 18 are each formed with a plurality of cuts 183.

The cuts 183 extend in the direction of the major axis J32, and are aligned in parallel to each other along an axial direction of the proximal separable piece 33.

In addition, the interval of the adjacent cuts 183 decreases gradually from the proximal side toward the distal side, which helps ensure that the degree of flexibleness of the flexible portion 18 increases gradually from the proximal side toward the distal side.

Note that the flexible portions 17 and 19 are also formed with cuts (not illustrated).

According to the twentieth embodiment as above, also, the same or equivalent effects to those of the aforementioned nineteenth embodiment can be produced.

Note that the twentieth embodiment is applicable also to the eighteenth embodiment and a twenty-first embodiment.

FIG. 65 is a perspective view showing a medical tube according to a twenty-first embodiment of the present disclosure. Note that in the following, for convenience of explanation, the right side in FIG. 65 will be referred to as “distal side,” the left side as “proximal side,” the upper side as “upper side,” and the lower side as “lower side.” In FIG. 65, for convenience of explanation, a sheath (medical tube) extending in a circular arc shape is depicted in the state of being stretched rectilinearly. Further, in FIG. 65, for convenience of explanation, a state maintaining mechanism and the like are omitted from the drawing.

Referring to this figure, the twenty-first embodiment of a medical tube assembly will be described below. The following description will center on differences from the aforementioned eighteenth embodiment, and descriptions of the same items as above will be omitted.

As depicted in FIG. 65, in a sheath 30 in this embodiment, a main body 31 is in the form of a single tube. The main body 31 is provided at a proximal end portion thereof with a flexible portion 19 more flexible than the proximal end portion. The flexible portion 19 is a part that is the most flexible of the main body 31 (the flexible portion 19 is more flexible than a part, on a central portion side of the flexible portion 19, of the main body 31). In accordance with an exemplary embodiment, the flexible portion 19 can include a pair of tongue pieces 191 and 192 being elastic, disposed opposite to each other with a center axis of the main body 31 therebetween, and elastically deformable in directions for spacing away from each other.

The tongue piece 191 and the tongue piece 192 of the flexible portion 19 are aligned in the direction of the major axis J32.

In addition, the flexible portion 19 has a part where the degree of flexibleness increases gradually from the side of a central portion of the main body 31 toward the side of the proximal end of the main body 31. In this embodiment, the thickness of the tongue piece 192 gradually decreases from the distal side toward the proximal side. Similarly, the thickness of the tongue piece 191 gradually decreases from the distal side toward the proximal side.

Note that the flexible portion 19 may be provided at a distal portion of the main body 31, or may be provided at both a distal portion and a proximal portion of the main body 31.

According to the twenty-first embodiment as above, also, the same or equivalent effects to those of the aforementioned eighteenth embodiment can be produced.

While the medical tube of the present disclosure has been described above based on the illustrated embodiments, the present disclosure is not limited to the embodiments. The configuration of each component can be replaced with an arbitrary configuration having the same or equivalent function. In addition, other arbitrary structure or structures may be added to the present disclosure.

In addition, while the needle body is retained on the main body in an attachable and detachable manner in the above embodiments, this configuration is not restrictive. For example, the needle body may be fixed to the main body, like in a configuration wherein the main body and the needle body are formed integrally. In this case, the distal-side opening of the main body can be opened by cutting the needle body by use of a pair of scissors or the like, after a living body is punctured by the puncture member and the needle body is protruded to the outside of the living body.

In accordance with an exemplary embodiment, the shape of the main body of the sheath is not limited to the shape in the above embodiments. For example, the main body may be rectilinear in shape, and the external shape in cross section of the main body may be a circle or the like.

Further, the main body of the sheath may be configured to be separable into the distal separable piece (first separable tube) and the proximal separable piece (second separable tube). Alternatively, the main body may be configured not to be separable to the distal side and the proximal side, that is, the main body may be in the form of a single tube.

In addition, the material constituting the main body of the sheath is not restricted to a rigid material. For example, a flexible material may also be used.

In accordance with an exemplary embodiment, the shape of the insertion section is not limited to the shape in the above embodiments. For instance, the insertion section may be rectilinear in shape.

In addition, while the sheath is configured as part of the puncture member in the above embodiments, this configuration is not restrictive. Specifically, a sheath may be used in the manner of being inserted into a penetrating hole preliminarily formed in a living body by use of some means. Specifically describing in correspondence with the aforementioned first embodiment, a puncture apparatus 1 with the puncture member 3 omitted therefrom is prepared, an insertion section 71 is used as a puncture member, and its distal portion 711 is made to puncture an inguinal region on the right side of the patient, to sequentially pass an obturator foramen on one side, between the urethra and the vagina, and an obturator foramen on the other side, and then to exit the living body via an inguinal region on the left side. Next, the insertion section 71 is inserted into the inside, and a sheath 30 (main body 31) is advanced into the body along the insertion section 71, resulting in a state where both ends of the sheath 30 are protruding from the body surface H. Subsequently, the insertion section 71 is drawn out of the body. As a result, the sheath 30 is disposed inside the living body. Then, an implant main body is disposed inside the sheath 30, and the sheath 30 is drawn out of the body, whereby the implant main body can be placed indwelling in the living body, like in the aforementioned embodiments.

In accordance with an exemplary embodiment, for example, the distal portion 711 of the insertion section 71 is made to puncture the inguinal region on the right side of the patient, to sequentially pass the obturator foramen on one side, between the urethra and the vagina, and the obturator foramen on the other side, and to protrude to the outside of the body via the inguinal region on the left side, and thereafter a distal portion of the sheath 30 is fixed to the distal portion 711. Next, the distal portion 711 is rotated in the opposite direction, to draw the insertion section 71 out of the body, and the sheath 30 is left indwelling in the living body. Then, the implant main body is disposed inside the sheath 30, and the sheath 30 is drawn out of the body, whereby the implant main body can be placed indwelling in the living body, like in the aforementioned embodiments.

In addition, while a configuration wherein the main body of the puncture member is disposed inside a living body and thereafter the implant main body is inserted into the main body has been described in the above embodiments, this configuration is not restrictive. A configuration may be adopted in which the implant main body is accommodated in the puncture member (main body) from the beginning. In this case, it is preferable that, for example, a string located on the needle tip side, of two strings possessed by the implant main body, is preliminarily fixed to the needle tip. This helps ensure that when the needle tip is detached from the main body, the string can be protruded to the outside of the main body in an attendant manner. As a result, the subsequent fine adjustment of the disposition of the implant main body and the like can be performed relatively smoothly.

In accordance with an exemplary embodiment, while a case where the puncture apparatus is applied to an apparatus for use in embedding in a living body an embeddable implant for treatment of female urinary incontinence has been described in the above embodiments, the use of the puncture apparatus is not limited to the described one.

For example, the target of the application of the present disclosure can include excretory disorders attendant on the weakening of the pelvic floor muscle group (urinary urgency, frequent urination, urinary incontinence, fecal incontinence, urinary retention, dysuria or the like), and pelvic floor disorders including pelvic organ prolapse, vesicovaginal fistula, urethrovaginal fistula, or pelvic pain. In the pelvic organ prolapse, there are included disorders of cystocele, enterocele, rectocele, and uterine prolapse. Alternatively, there are included such disorders as anterior vaginal prolapse, posterior vaginal prolapse, vaginal apical prolapse, vaginal vault prolapse and the like in which the naming method thereof is based on the prolapsed vaginal-wall part.

Also, overactive tissues can include bladder, vagina, uterus, and bowel. Lessactive tissues can include bones, muscles, fascias, and ligaments. In particular, in the case of pelvic floor disorders, the lessactive tissues include an obturator fascia, a coccygeus fascia, a cardinal ligament, an uterosacral ligament, and a sacrospinous ligament.

For the procedure for interlocking an overactive tissue in the pelvic floor disorder with the lessactive tissue, there are included a retropubic sling surgery, a transobturator sling surgery (transobturator tape (TOT) surgery), a tension-free vaginal mesh (TVM) surgery, a uterosacral ligament suspension (USLS) surgery, a sacrospinous ligament fixation (SSLF) surgery, an iliococcygeus fascia fixation surgery, and a coccygeus fascia fixation surgery.

The medical tube assembly according to the one aspect of the present disclosure can include a medical tube having a tubular main body in which an implant to be placed indwelling in a living body is insertable, an insertion section having a curved portion and being elongated, an interlock portion interlocking a distal portion of the medical tube and a distal portion of the insertion section in a freely detachable manner, and a rotation restriction unit restricting rotation of the medical tube and the insertion section around an axis, wherein when inserted into a living body with the distal portion of the medical tube and the distal portion of the insertion section interlocked to each other, the insertion section and the main body are inserted in an aligned state.

In addition, the puncture apparatus according to another aspect of the present disclosure can include the medical tube assembly of one aspect of the present disclosure, the medical tube assembly disposed to be rotationally movable and provided at a distal portion thereof with a needle body capable of puncturing a living body, a urethral-insertion member to be inserted into a urethra, the urethral-insertion member elongated in shape, and a restriction unit restricting positional relationship of the medical tube assembly and the urethral-insertion member in such a manner that when the medical tube assembly is rotationally moved for puncturing a biological tissue, a needle point of the needle body passes on a farther side from a center of rotary movement of the medical tube assembly than the urethral-insertion member.

According to the described aspects of the present disclosure, the medical tube assembly can include the interlock portion interlocking the distal portion of the medical tube and the distal portion of the insertion section to each other and the rotation restriction unit. By interlocking the distal portion of the medical tube and the distal portion of the insertion section, therefore, the procedure can be performed without inserting the insertion section into the medical tube. Accordingly, the length of the medical tube can be set greater than the length of the insertion section, so that the medical tube assembly can be applied, for example, to a fat patient. In other words, the implant can be placed indwelling in a living body easily and reliably, in the cases of patients of various body types. Therefore, the medical tube assembly according to the present disclosure has industrial applicability.

The detailed description above describes a medical tube assembly and a puncture apparatus. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical tube assembly comprising:
a medical tube having a tubular main body in which an implant to be placed indwelling in a living body is insertable;
an insertion section having a curved portion and being elongated;
an interlock portion interlocking a distal portion of the medical tube and a distal portion of the insertion section in a freely detachable manner; and
a rotation restriction unit restricting rotation of the medical tube and the insertion section around an axis,
wherein when inserted into a living body with the distal portion of the medical tube and the distal portion of the insertion section interlocked to each other, the insertion section and the main body are inserted in an aligned state.

2. The medical tube assembly according to claim 1, wherein a length of the main body in an axial direction is greater than a length of the insertion section in an axial direction.

3. The medical tube assembly according to claim 1, wherein the rotation restriction unit is provided at distal portions of the main body and the insertion section.

4. The medical tube assembly according to claim 1, wherein the rotation restriction unit is provided at side surfaces of the main body and the insertion section.

5. The medical tube assembly according to claim 1,
wherein the main body has a curved portion, and
the main body, in a state of being inserted in a living body, is rigid in such a manner as to be able to maintain curvature and an internal cavity of the main body.

6. The medical tube assembly according to claim 1, further comprising
a needle body capable of puncturing a living body at a distal portion of the medical tube assembly.

7. A puncture apparatus comprising:
a medical tube assembly disposed to be rotationally movable, the medical tube assembly including a medical tube having a tubular main body in which an implant to be placed indwelling in a living body is insertable, an insertion section having a curved portion and being elongated, an interlock portion interlocking a distal portion of the medical tube and a distal portion of the insertion section in a freely detachable manner, a rotation restriction unit restricting rotation of the medical tube and the insertion section around an axis, and a needle body capable of puncturing a living body at a distal portion of the medical tube assembly, when inserted into a living body with the distal portion of the medical tube and the distal portion of the insertion section interlocked to each other, the insertion section and the main body being inserted in an aligned state;
a urethral-insertion member to be inserted into a urethra, the urethral-insertion member elongated in shape; and
a restriction unit restricting positional relationship of the medical tube assembly and the urethral-insertion member in such a manner that when the medical tube assembly is rotationally moved for puncturing a biological tissue, a needle point of the needle body passes on a farther side from a center of rotary movement of the medical tube assembly than the urethral-insertion member.

8. The puncture apparatus according to claim 7,
wherein the main body is flat shaped in cross section, and
the rotation restriction unit restricts the rotation in such a manner that a major axis in cross section of the main body and an axis of the urethral-insertion member will be parallel to each other.

9. The puncture apparatus according to claim 7, wherein a length of the main body in an axial direction is greater than a length of the insertion section in an axial direction.

10. The puncture apparatus according to claim 7, wherein the rotation restriction unit is provided at distal portions of the main body and the insertion section.

11. The puncture apparatus according to claim 7, wherein the rotation restriction unit is provided at side surfaces of the main body and the insertion section.

12. The puncture apparatus according to claim 7,
wherein the main body has a curved portion, and
the main body, in a state of being inserted in a living body, is rigid in such a manner as to be able to maintain curvature and an internal cavity of the main body.

13. A method of forming a path in living body tissue, the method comprising:
inserting a urethral-insertion member into a urethra, the urethral-insertion member elongated in shape;
inserting a medical tube assembly into a living body, the medical tube assembly configured to be rotationally movable, the medical tube assembly including a medical tube having a tubular main body in which an implant to be placed indwelling, an insertion section having a curved portion and being elongated, an interlock portion interlocking a distal portion of the medical tube and a distal portion of the insertion section in a freely detachable manner, a rotation restriction unit restricting rotation of the medical tube and the insertion section around an axis, and a needle body capable of puncturing a living body at a distal portion of the medical tube assembly, when inserted into a living body with the distal portion of the medical tube and the distal portion of the insertion section interlocked to each other, the insertion section and the main body being inserted in an aligned state; and
restricting positional relationship of the medical tube assembly and the urethral-insertion member with the rotation restriction unit in such a manner that when the medical tube assembly is rotationally moved for puncturing a biological tissue, a needle point of the needle body passes on a farther side from a center of rotary movement of the medical tube assembly than the urethral-insertion member.

14. The method according to claim 13,
wherein the main body is flat shaped in cross section, and
restricting the rotation with the rotation restriction unit in such a manner that a major axis in cross section of the main body and an axis of the urethral-insertion member will be parallel to each other.

15. The method according to claim 13, wherein a length of the main body in an axial direction is greater than a length of the insertion section in an axial direction.

16. The method according to claim 13, comprising:
providing the rotation restriction unit at distal portions of the main body and the insertion section.

17. The method according to claim 13, comprising:
providing the rotation restriction unit at side surfaces of the main body and the insertion section.

18. The method according to claim 13,
wherein the main body has a curved portion, and
the main body, in a state of being inserted in a living body, is rigid in such a manner as to be able to maintain curvature and an internal cavity of the main body.
